(19) United States
(12) Patent Application Publication
(10) Pub. No.: US 2021/0322642 A1
(43) Pub. Date: Oct. 21, 2021

(54) METHODS OF LIVER RECELLULARIZATION

(71) Applicant: Miromatrix Medical Inc., Eden Prairie, MN (US)

(72) Inventors: Jeffrey ROSS, Eden Prairie, MN (US); Dominique Seetapun DAVIDOW, Eden Prairie, MN (US); Ben STEINER, Eden Prairie, MN (US)

(21) Appl. No.: 17/111,577

(22) Filed: Dec. 4, 2020

Related U.S. Application Data
(63) Continuation of application No. PCT/US2019/035449, filed on Jun. 4, 2019.
(60) Provisional application No. 62/680,253, filed on Jun. 4, 2018.

(51) Int. Cl. A61L 27/38; A61L 27/36; A01N 1/02

(52) U.S. Cl. CPC A61L 27/3808; A61L 27/3633; A61L 2430/28; A01N 1/0252; A61L 27/3683

(57) ABSTRACT

Disclosed herein are recellularized livers prepared from decellularized liver extracellular matrices. Also disclosed herein are kits and systems comprising a recellularized liver as described herein. Also disclosed herein are methods of recellularizing livers from decellularized liver extracellular matrices.

### CROSS-REFERENCE

This application is a continuation of International Application No. PCT/US2019/035449, filed Jun. 4, 2019, which claims the benefit of U.S. Provisional Application No. 62/680,253, filed Jun. 4, 2018, both are which incorporated by reference herein in their entirety.

### SUMMARY

Disclosed herein are at least partially recellularized livers that can comprise a perfusion decellularized extracellular matrix from a first animal and a population of engrafted endothelial cells from a second animal, where following engraftment the engrafted endothelial cells within the sinusoidal compartment develop a fenestration phenotype.

Also disclosed herein are at least partially recellularized livers that can comprise a fenestrated endothelium, a perfusion decellularized extracellular matrix from a first animal, and a plurality of endothelial cells from a second animal engrafted thereon, where the fenestration comprises cells differentiated from the plurality of endothelial cells.

Also disclosed herein are at least partially recellularized livers that can comprise a perfusion decellularized extracellular matrix from a first animal and a plurality of endothelial cells from a second animal; where prior to the recellularization, the perfusion decellularized liver included a non-vasculature decellularized extracellular matrix and a vasculature decellularized extracellular matrix, and where the endothelial cells engraft, migrate and/or proliferation into a parenchymal or sinusoidal niche.

Also disclosed herein are at least partially recellularized livers that can comprise a perfusion decellularized extracellular matrix from a first animal and a plurality of endothelial cells from a second animal engrafted thereon; wherein prior to the recellularization, the perfusion decellularized liver included a non-vasculature decellularized extracellular matrix and a vasculature decellularized extracellular matrix, and wherein the at least partially recellularized liver comprises a greater expression level of LYVE-1 in a parenchymal niche of the at least partially recellularized liver relative to an expression level of LYVE-1 in a large vessel of the at least partially recellularized liver, as determined by isolating extraction of RNA from tissue of the at least partially recellularized liver and quantitative reverse-transcriptase PCR. In some embodiments, an at least partially recellularized liver can comprise a greater expression level of STAB-2 in a parenchymal niche of an at least partially recellularized liver relative to an expression level of STAB-2 in a large vessel of an at least partially recellularized liver, as determined by isolating extraction of RNA from tissue of an at least partially recellularized liver and quantitative reverse-transcriptase PCR.

In some embodiments, a perfusion decellularized matrix can comprise a substantially intact exterior surface. In some embodiments, a first animal can be a mammal. In some embodiments, a mammal can be a rodent, a pig, a monkey, a rabbit, a cow, a goat, a sheep, a dog, or a human. In some embodiments, a mammal can be a human. In some embodiments, a second animal can be a mammal. In some embodiments, a mammal can be a rodent, a pig, a monkey, a rabbit, a cow, a goat, a sheep, a dog, or a human. In some embodiments, a mammal can be a human. In some embodiments, endothelial cells can be human umbilical vein endothelial cells (HUVEC). In some embodiments, an at least partially recellularized liver can further comprise a cannula. In some embodiments, an at least partially recellularized liver in media can have a 24 hour glucose consumption level of at least about 10 mg/hr, as determined by collecting a media and measuring a level of glucose using an electrochemical sensor.

Also disclosed herein are kits that can comprise an at least partially recellularized liver as described herein in a sterile container.

Also disclosed herein are systems that can comprise an at least partially recellularized liver as described herein, an input attached to an at least partially recellularized liver, an output attached to an at least partially recellularized liver, growth media, and at least one of: a temperature control apparatus, an atmosphere controlling apparatus, or a humidity controlling apparatus.

Also disclosed herein are cleanrooms that can comprise an at least partially recellularized liver as described herein, a kit as described herein, or a system as described herein.

Also disclosed herein are factories that can comprise an at least partially recellularized liver as described herein, a kit as described herein, a system as described herein, or a cleanroom as described herein.

Also disclosed herein are methods that can comprise transplanting an at least partially recellularized liver as described herein.

Also disclosed herein are methods that can comprise implanting an at least partially recellularized liver as described herein.

Also disclosed herein are methods that can comprise providing a perfusion decellularized extracellular matrix of a decellularized mammalian liver in media, introducing a first solution that can comprise a population of endothelial cells to a perfusion decellularized extracellular matrix; such that at least some of a population of endothelial cells engraft on at least a portion of the perfusion decellularized extracellular matrix, thereby providing a recellularized extracellular matrix of the decellularized mammalian liver, measuring a 24 hour glucose consumption level in a media of endothelial cells engrafted on a recellularized extracellular matrix, and transplanting an recellularized extracellular matrix into a recipient when the 24 hour glucose level is at least about 10 mg/hr.

Also disclosed herein are methods that can comprise: administering to a recipient an immunosuppressor; introducing a first solution that can comprise a population of endothelial cells to a perfusion decellularized extracellular matrix; such that at least some of a population of endothelial cells engraft on at least a portion of a perfusion decellularized extracellular matrix, thereby providing a recellularized extracellular matrix of a decellularized mammalian liver; and transplanting a reendothelialized liver matrix into a recipient. In some embodiments, a method can reduce thrombogenesis and immunogenicity in a recellularized liver following transplantation into a recipient. In some embodiments, an immunosuppressor can be a corticosteroid. In some embodiments, a corticosteroid can be methylprednisolone. In some embodiments, an administering of an immunosuppressor can be once a day. In some embodiments, an administering can be oral or parenteral. In some embodiments, an administering can be parenteral. In some embodiments, a parenteral administering can be intravenous.

In some embodiments, an endothelial cell can be autologous to a recipient. In some embodiments, am endothelial cell can be allogeneic to the recipient. In some embodiments, an endothelial cell can be from a mammal. In some embodiments, a mammal can be a rodent, a pig, a monkey, a rabbit, a cow, a goat, a sheep, a dog, or a human. In some embodiments, a mammal can be a human. In some embodiments, a recipient can be a mammal. In some embodiments, a mammal can be a rodent, a pig, a monkey, a rabbit, a cow, a goat, a sheep, a dog, or a human. In some embodiments, a mammal can be a human. In some embodiments, a population of endothelial cells can be a population of human umbilical vein endothelial cells (HUVEC). In some embodiments, an introducing can comprise perfusing into a vessel, a duct, or a cavity of a perfusion decellularized extracellular matrix. In some embodiments, an introducing can comprise injecting into a perfusion decellularized extracellular matrix. In some embodiments, a method can further comprise introducing a second solution into a perfusion decellularized extracellular matrix prior to a transplanting. In some embodiments, a method can further comprise introducing a third solution into a perfusion decellularized extracellular matrix prior to a transplanting. In some embodiments, a recellularized extracellular matrix can remain patent after a transplanting for at least about 10 days, as determined by measurement of blood flow rate across a recellularized extracellular matrix.

Also disclosed herein are methods of quality testing a recellularized liver, that can comprise: providing a recellularized liver, where a recellularized liver can comprise a perfusion decellularized extracellular matrix and a population of endothelial cells engrafted thereon; determining a presence of a fenestration on a recellularized liver; detecting a level of glucose consumption within a 24 hour period; and designating a recellularized liver for further manufacture if a recellularized liver has a fenestration and a level of glucose consumption within a 24 hour period of at least about 10 mg/hr.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1A-1P depict in vitro characterization of Human Umbilical Vein Endothelial Cell (HUVEC) seeded liver grafts and metabolic characterization of glucose consumption. FIG. 1A depicts decellularized whole porcine liver grafts. FIG. 1B depicts characterization of intact matrix by H&E. FIG. 1C depicts Collagen I staining. FIG. 1D depicts a representative image of a whole liver bioreactor with a re-endothelialized liver graft. FIG. 1E depicts daily glucose consumption curves showing the increasing metabolic activity of the liver grafts defined by early, mid and peak glucose consumption rates (PGCR). FIGS. 1F-1H show glucose consumption rates correlate to endothelial cell coverage as characterized by H&E. FIGS. 1I-1K show that glucose consumption rates correlate to endothelial cell coverage as characterized by CD-31 and Collagen I staining. FIGS. 1F-1K demonstrate a dose-dependent response allowing for a non-destructive measurement of endothelial cell growth within the liver grafts. FIGS. 1L-1O depict staining with LYVE1 and CD-31, which demonstrate a significant increase in LYVE1 detection within the sinusoidal space compared to earlier phases with stronger CD31 expression to larger vessels. FIG. 1P depicts analysis of endothelial gene expression for vascular markers CD-31 and VE-Cadherin and sinusoidal markers LYVE1 and STAB2, normalized to 2-D HUVEC culture expression, which demonstrate a significant increase in sinusoidal gene expression from early to late phases.

FIGS. 2A-2D depict a summary of short-term studies. FIG. 2A depicts the total number and break down of grafts utilized accompanied by schematic demonstrating of the in vitro and in vivo models. FIGS. 2B-2C depict in vitro metabolic profiles of bioengineered grafts during incubation and their ability to predict sustained short-term blood flow in-vitro and in-vivo. FIG. 2C shows that Peak (PGCR) and End glucose consumption rate (EGCR) are good markers to predict subsequent percentile blood flow during short term studies (*P<0.01). FIG. 2D depicts histopathological evaluation of endothelialized grafts after testing. Noted blood sequestration in grafts with low PGCR (<20 mg/hr) compared to High PGCR (>30).

FIGS. 3A-3D depict a summary of long-term pre-clinical studies. FIG. 3A depicts live pictures of the steps of the surgical procedure and the following intra-operative ultrasound of graft's portal vein and hepatic veins. FIG. 3B depicts the total number and break down of grafts utilized. FIG. 3C depicts an example of gradual loss of graft perfusion in the non-immunosuppressed group. Note minimal perfusion at day 7 and complete thrombosis by day 10. FIG. 3D depicts a reconstructed CT scan of a pig after graft implantation.

FIGS. 4A-4F demonstrating the negative effect of the host's immune response towards the implanted HUVECs seeded grafts. FIGS. 4A-4B and 4D-4E demonstrate the changes in immunological profile and the accompanying effects on HUVEC seeded graft perfusion with and without immune suppression. FIGS. 4C and 4F are serial contrast enhanced CT images of the implanted bioengineered liver grafts over time. Grafts are highlighted with dotted lines. Red dotted lines reflect thrombosed graft with no parenchymal perfusion. FIG. 4G depicts histological examination of an implanted graft in immunosuppressed animal after in-vivo perfusion for 7 days. Preserved viability of the cells constructing the vascular network was demonstrated as reflected by the CD31 staining.

FIGS. 5A-5I depict a porcine liver decellularization and perfusion bioreactor system. FIG. 5A is an illustration showing native porcine livers are cannulated on the PV, IVC and SVC, and decellularized by sequential perfusion with Triton X-100 and SDS solutions. FIGS. 5B-5G are representative photographs, H&E staining and Collagen I immunofluorescence of native (FIGS. 5B-5D) and decellularized (FIGS. 5E-5G) porcine livers. FIGS. 5H and 5I depict a schematic (FIG. 5H) and photo (FIG. 5I) of perfusion bioreactor system comprised of a custom bioreactor and a pressure-dependent perfusion control system. The bioreactor includes a pressure transducer (PT) to monitor perfusion pressure, a gas exchange coil (GEC) to allow efficient gas exchange during media perfusion, a bubble trap (BT) to prevent the introduction of bubbles into the rBEL, a 0.22 μm filter air vent (AV), and three-way stopcocks (3W) to enable media exchange and sampling.

FIGS. 6A-6T depict analysis of rBEL culture kinetics and HUVEC phenotypic plasticity in decellularized liver matrix. FIG. 6A shows HUVECs are expanded in 2D tissue culture flasks, harvested and seeded through the graft SVC, followed by the PV 24 hours later. FIG. 6B shows representative CD31+ flow cytometry demonstrating a phenotypically pure HUVEC population immediately prior to graft seeding. FIG. 6C shows plots of glucose consumption rates over time from independently seeded and cultured rBEL constructs. Glucose consumption rates correlated with total endothelial cell coverage as characterized by H&E staining (FIGS. 6D-6F) and anti-CD31 immunostaining (FIGS. 6G-6I). FIG. 6J depicts quantitative RT-PCR analysis of CD31, LYVE1 and STAB2 mRNA levels in rBELs harvested at different phases of glucose consumption kinetics. Data are plotted as fold change relative to HUVECs in 2D culture. Error bars indicate S.E.M. between biological replicates. FIG. 6K-6M depict CD31 and LYVE-1 immunostaining from rBELs harvested at low, mid and high glucose consumption phases. FIG. 6N depicts principal component analysis of RNA-seq gene expression profiles from rBELs harvested at low and high glucose consumption phases. FIG. 6O depicts similarity matrix comparing low and high glucose consumption phase rBEL samples with respect to panel of known liver endothelial cell markers (input genes: F8, CD31, STAB2, LYVE1, CD14, VWF, ENG, ICAM1). FIG. 6p depicts RNA-seq expression profiles for liver sinusoidal endothelial markers LYVE1, VWF, and ICAM1 in low and high glucose consumption phase rBEL samples. HUVECs and primary human LSECs cultured in 2D are included for comparison. FIG. 6Q-6T depicts TEM images from native liver (FIG. 6Q) and rBEL (FIGS. 6R-6T) samples. Red arrows indicate fenestrae-like structures within endothelial cells.

FIG. 7A-7K depict in vitro and in vivo patency correlates with peak glucose consumption rate. FIGS. 7A and 7C depict a diagram and setup of the in vitro blood circuit used to evaluate rBEL patency. The circuit perfuses a rBEL with warm, heparinized whole porcine blood and is driven by a peristaltic pump controlled by a pressure-based custom control system. FIG. 7B shows an illustration of in vivo heterotopic liver implant model where the rBEL is anastomosed via the PV and IVC to the native PV and IVC. Partial flow was given to both the rBEL and the native liver by restricting flow to the native liver. FIG. 7D-7I are representative images of the heterotopic liver implant including graft preparation, anastomosis and reperfusion. FIG. 7J are representative ultrasound images of an implanted rBEL demonstrating portal and hepatic veins flow after 30 min. FIG. 7K depicts that peak glucose consumption of >30 mg/h correlates with >100 mL/min of blood flow in vitro and in vivo.

FIG. 8A-8E depict long term in vivo perfusion studies in the presence and absence of immunosuppression. FIG. 8A shows in vivo implants were separated into two groups: no treatment and immunosuppression. The immunosuppression group received a methylprednisolone immunosuppression dose (I.D.) starting at 500 mg on Day 0 and was tapered over ten days. FIG. 8B is a 3D CT reconstruction after graft implantation showing the heterotopic position of the implanted graft below the native liver while demonstrating good vascular perfusion of the implanted graft. FIG. 8C shows serial contrast enhanced CT images of the implanted bioengineered liver grafts over time. Grafts are highlighted with dotted lines. Yellow dotted lines delineate perfused graft with contrast in white. Red dotted lines reflect no parenchymal perfusion. FIG. 8D depicts quantification of graft perfusion reduction over time exhibiting an extension of perfusion over time with immunosuppression. FIG. 8E shows cytotoxicity of pig sera incubated on in vitro HUVEC cultures following addition of unabsorbed rabbit complement.

FIG. 9 depicts histological comparison of blood sequestration in representative rBELs following explant from animals used in acute blood flow studies.

FIGS. 10A-10C depicts histological assessment of rBEL explanted from immunosuppressed animal at 7 days post implantation. (FIG. 10A) H&E staining, (FIG. 10B) anti-CD31 immunostaining, and (FIG. 10C) anti-C4D immunostaining from explanted rBEL histological sections.

FIGS. 11A-11C depict quantification of graft perfusion over time by CT volumetry. Analysis of graft volumetry measurements from long-term implant studies showing total graft volume (FIG. 11A), total perfused volume (FIG. 11B), and relative percent perfusion (FIG. 11C).

### DETAILED DESCRIPTION

### I. Overview

Disclosed herein are methods of preparing at least partially recellularized livers. In some instances, an at least partially recellularized liver can be allogeneic. Allogeneic whole organ transplantation is curative for end stage organ failure, but limits on the supply of donor organ material, immunological incompatibility, and coagulopathy remain significant translational barriers. Development of a bioengineered liver would provide an alternative to allogeneic transplant, but have been limited by the ability to reconstitute functional revascularization. Embodiments described herein demonstrate engraftment of functional endothelial cells, as well as proliferation and migration of endothelial cells into the parenchymal space, thereby demonstrating successful liver recellularization and further transformation into sinusoidal or sinusoidal like endothelial cells.

Accordingly, disclosed herein are at least partially recellularized livers that can comprise a perfusion decellularized extracellular matrix from a first animal and a population of engrafted endothelial cells from a second animal, where following engraftment the engrafted endothelial cells within the sinusoidal compartment develop a fenestration phenotype.

Also disclosed herein are at least partially recellularized livers that can comprise a fenestrated endothelium, a perfusion decellularized extracellular matrix from a first animal, and a plurality of endothelial cells from a second animal engrafted thereon, where the fenestration comprises cells differentiated from the plurality of endothelial cells.

Also disclosed herein are at least partially recellularized livers that can comprise a perfusion decellularized extracellular matrix from a first animal and a plurality of endothelial cells from a second animal; where prior to the recellularization, the perfusion decellularized liver included a non-vasculature decellularized extracellular matrix and a vasculature decellularized extracellular matrix, and where the endothelial cells engraft, migrate and/or proliferation into a parenchymal or sinusoidal niche.

Also disclosed herein are at least partially recellularized livers that can comprise a perfusion decellularized extracellular matrix from a first animal and a plurality of endothelial cells from a second animal engrafted thereon; wherein prior to the recellularization, the perfusion decellularized liver included a non-vasculature decellularized extracellular matrix and a vasculature decellularized extracellular matrix, and wherein the at least partially recellularized liver comprises a greater expression level of LYVE-1 in a parenchymal niche of the at least partially recellularized liver relative to an expression level of LYVE-1 in a large vessel of the at least partially recellularized liver, as determined by isolating extraction of RNA from tissue of the at least partially recellularized liver and quantitative reverse-transcriptase PCR.

Also disclosed herein are kits that can comprise an at least partially recellularized liver as described herein in a sterile container.

Also disclosed herein are systems that can comprise an at least partially recellularized liver as described herein, an input attached to an at least partially recellularized liver, an output attached to an at least partially recellularized liver, growth media, and at least one of: a temperature control apparatus, an atmosphere controlling apparatus, or a humidity controlling apparatus.

Also disclosed herein are cleanrooms that can comprise an at least partially recellularized liver as described herein, a kit as described herein, or a system as described herein.

Also disclosed herein are factories that can comprise an at least partially recellularized liver as described herein, a kit as described herein, a system as described herein, or a cleanroom as described herein.

Also disclosed herein are methods that can comprise transplanting an at least partially recellularized liver as described herein.

Also disclosed herein are methods that can comprise implanting an at least partially recellularized liver as described herein.

Also disclosed herein are methods that can comprise providing a perfusion decellularized extracellular matrix of a decellularized mammalian liver in media, introducing a first solution that can comprise a population of endothelial cells to a perfusion decellularized extracellular matrix; such that at least some of a population of endothelial cells engraft on at least a portion of the perfusion decellularized extracellular matrix, thereby providing a recellularized extracellular matrix of the decellularized mammalian liver, measuring a 24 hour glucose consumption level in a media of endothelial cells engrafted on a recellularized extracellular matrix, and transplanting an recellularized extracellular matrix into a recipient when the 24 hour glucose level is at least about 10 mg/hr.

Also disclosed herein are methods that can comprise: administering to a recipient an immunosuppressor; introducing a first solution that can comprise a population of endothelial cells to a perfusion decellularized extracellular matrix; such that at least some of a population of endothelial cells engraft on at least a portion of a perfusion decellularized extracellular matrix, thereby providing a recellularized extracellular matrix of a decellularized mammalian liver; and transplanting a reendothelialized liver matrix into a recipient.

Also disclosed herein are methods of quality testing a recellularized liver, that can comprise: providing a recellularized liver, where a recellularized liver can comprise a perfusion decellularized extracellular matrix and a population of endothelial cells engrafted thereon; determining a presence of a fenestration on a recellularized liver; detecting a level of glucose consumption within a 24 hour period; and designating a recellularized liver for further manufacture if a recellularized liver has a fenestration and a level of glucose consumption within a 24 hour period of at least about 10 mg/hr.

### II. Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

The term “about” and its grammatical equivalents in relation to a reference numerical value and its grammatical equivalents as used herein may include a range of values plus or minus 10% from that value. For example, the amount “about 10” includes amounts from 9 to 11. The term “about” in relation to a reference numerical value may also include a range of values plus or minus: 10%, 9%, 8%, 7%, 6%, 5%, 4%, 3%, 2%, or 1% from that value.

The term “substantially” as used herein may refer to a value approaching 100% of a given value. In some embodiments, the term may refer to an amount that may be at least about 90%, 91%, 92%, 93%, 94%, 95%, 96%, 97%, 98%, 99%, 99.9%, or 99.99% of a total amount. In some embodiments, the term may refer to an amount that may be about 100% of a total amount.

The term “decellularized” or “decellularization” as used herein may refer to a biostructure (e.g., an isolated organ or portion thereof, or tissue), from which the cellular and tissue content has been reduced or removed leaving behind an intact acellular infra-structure. Organs such as the kidney can be composed of various specialized tissues. Specialized tissue structures of an organ, or parenchyma, can provide specific function associated with the organ. Supporting fibrous network of an isolated organ can be a stroma. Most organs have a stromal framework composed of unspecialized connecting tissue which supports the specialized tissue. The process of decellularization may at least partially remove the cellular portion of the tissue, leaving behind a complex three-dimensional network of extracellular matrix (ECM). An ECM infrastructure may primarily be composed of collagen but can include cytokines, proteoglycans, laminin, fibrillin and other proteins secreted by cells. An at least partially decellularized structure may provide a biocompatible substrate onto which different cell populations may be infused or used to be implanted as acellular medical devices such as but not limited to, wound care matrix, fistula matrix, void filler, dermal fillers, soft tissue reinforcement, or other substrates that enable cellular infiltration and remodeling following implantation or application. Decellularized biostructures may be rigid, or semi-rigid, having an ability to alter their shapes. Examples of decellularized isolated organs may include, but are not limited to solid organs such as, a heart, kidney, liver, lung, pancreas, brain, bone, spleen, gall bladder, urinary bladder, uterus, ureter, and urethra.

The term “recellularize” or “recellularization” as used herein may refer to an engraftment or distribution of a plurality of cells as described herein onto a decellularized extracellular matrix. A recellularized organ may comprise morphology or activity of a native, non-decellularized organ.

The term “effective amount” or “therapeutically effective amount” may refer to a quantity of a composition, for example a composition comprising cells such as cells, that can be sufficient to result in a desired activity upon introduction into an isolated organ or portion thereof disclosed herein.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

The term “recipient” and their grammatical equivalents as used herein may refer to a subject. A subject may be a human or non-human animal. A recipient may also be in need thereof, such as needing treatment for a disease such as cancer. In some embodiments, a recipient may be in need thereof of a preventative therapy. A recipient may not be in need thereof in other cases.

The term “subject” and its grammatical equivalents as used herein may refer to a human or a non-human. A subject may be a mammal. A subject may be a human mammal of a male or female biological gender. A subject may be of any age. A subject may be an embryo. A subject may be a newborn or up to about 100 years of age. A subject may be in need thereof. A subject may have a disease such as cancer. A subject may be premenopausal, menopausal, or have induced menopause.

The terms “treatment” or “treating” and their grammatical equivalents may refer to the medical management of a subject with an intent to cure, ameliorate, stabilize, or prevent a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include preventative treatment, that is, treatment directed to minimizing or partially or completely inhibiting the development of a disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure, ameliorate, stabilize or prevent a disease, condition, or disorder.

### III. Organ Decellularization

Disclosed herein are at least partially recellularized livers or portions thereof, prepared from a decellularized extracellular matrix. Decellularization can be performed using methods described in U.S. Pat. No. 8,470,520, which is incorporated by reference herein in its entirety.

The initial step in decellularizing an organ or tissue, such as a liver, is to cannulate the organ or tissue, if possible. The vessels, ducts, and/or cavities of an organ or tissue can be cannulated using methods and materials known in the art. The next step in decellularizing an organ or tissue is to perfuse the cannulated organ or tissue with a cellular disruption medium. Perfusion through an organ can be multi-directional (e.g., antegrade and retrograde). Langendorff perfusion of a heart is routine in the art, as is physiological perfusion (also known as four chamber working mode perfusion). See, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988. Briefly, for Langendorff perfusion, the aorta is cannulated and attached to a reservoir containing cellular disruption medium. A cellular disruption medium can be delivered in a retrograde direction down the aorta either at a constant flow rate delivered, for example, by an infusion or roller pump or by a constant hydrostatic pressure. In both instances, the aortic valves are forced shut and the perfusion fluid is directed into the coronary ostia (thereby perfusing the entire ventricular mass of the heart), which then drains into the right atrium via the coronary sinus. For working mode perfusion, a second cannula is connected to the left atrium and perfusion can be changed from retrograde to antegrade.

Methods are known in the art for perfusing other organ or tissues. By way of example, the following references describe the perfusion of lung, liver, kidney, brain, and limbs. Van Putte et al., 2002, Ann. Thorac. Surg., 74(3):893-8; den Butter et al., 1995, Transpl. Int., 8:466-71; Firth et al., 1989, Clin. Sci. (Lond.), 77(6):657-61; Mazzetti et al., 2004, Brain Res., 999(1):81-90; Wagner et al., 2003, J. Artif. Organs, 6(3):183-91.

One or more cellular disruption media can be used to decellularize an organ or tissue. A cellular disruption medium generally includes at least one detergent such as SDS, PEG, or Triton X. A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collegenase inhibitors).

In certain embodiments, a cannulated organ or tissue can be perfused sequentially with two different cellular disruption media. For example, the first cellular disruption medium can include an anionic detergent such as SDS and the second cellular disruption medium can include an ionic detergent such as Triton X. Following perfusion with at least one cellular disruption medium, a cannulated organ or tissue can be perfused, for example, with wash solutions and/or solutions containing one or more enzymes such as those disclosed herein. Alternating the direction of perfusion (e.g., antegrade and retrograde) can help to effectively decellularize the entire organ or tissue. Decellularization as described herein essentially decellularizes the organ from the inside out, resulting in very little damage to the ECM. An organ or tissue can be decellularized at a suitable temperature between 4 and 40° C. Depending upon the size and weight of an organ or tissue and the particular detergent(s) and concentration of detergent(s) in the cellular disruption medium, an organ or tissue generally is perfused from about 0.1 to about 12 hours per gram of solid organ or tissue with cellular disruption medium. Including washes, an organ may be perfused for up to about 12 to about 72 hours per gram of tissue. Perfusion generally is adjusted to physiologic conditions including pulsatile flow, rate and pressure.

As disclosed herein, a decellularized organ or tissue consists essentially of the extracellular matrix (ECM) component of all or most regions of the organ or tissue, including ECM components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

To effectively recellularize and generate an organ or tissue, it is important that the morphology and the architecture of the ECM be maintained (i.e., remain substantially intact) during and following the process of decellularization. “Morphology” as used herein can refer to the overall shape of the organ or tissue or of the ECM, while “architecture” as used herein can refer to the exterior surface, the interior surface, and the ECM therebetween.

The morphology and architecture of the ECM can be examined visually and/or histologically. For example, the basal lamina on the exterior surface of a solid organ or within the vasculature of an organ or tissue should not be removed or significantly damaged due to decellularization. In addition, the fibrils of the ECM should be similar to or significantly unchanged from that of an organ or tissue that has not been decellularized.

One or more compounds can be applied in or on a decellularized organ or tissue to, for example, preserve the decellularized organ, or to prepare the decellularized organ or tissue for recellularization and/or to assist or stimulate cells during the recellularization process. Such compounds include, but are not limited to, one or more growth factors (e.g., VEGF, DKK-1, FGF, bFGF, PDGF, HGF, BMP-1, BMP-4, SDF-1, IGF, and HGF), immune modulating agents (e.g., cytokines, glucocorticoids, IL2R antagonist, leucotriene antagonists, including but not limited to antibody therapy, use of stem cells to modulate the immune response, bone marrow transplant, etc.), and/or factors that modify the coagulation cascade (e.g., aspirin, heparin-binding proteins, and heparin). In addition, a decellularized organ or tissue can be further treated with, for example, irradiation (e.g., UV, gamma) to reduce or eliminate the presence of any type of microorganism remaining on or in a decellularized organ or tissue.

In some aspects, perfusion decellularization of an ECM from an organ or tissue can retain a native microstructure, such as an intact vascular and/or microvascular system, as compared to other decellularization techniques such as immersion based decellularization. For example, perfusion decellularized ECM from organs or tissues can preserve the collagen content and other binding and signaling factors and vasculature structure, thus providing for a niche environment with native cues for functional differentiation or maintenance of cellular function of introduced cells. In one embodiment, perfusion decellularized ECM from organs or tissues can be perfused with cells and/or media using the vasculature of the perfusion decellularized ECM under appropriate conditions, including appropriate pressure and flow to mimic the conditions normally found in the organism. The normal pressures of human sized organs can be between about 40 mm Hg to about 200 mm Hg with the resulting flow rate dependent upon the incoming perfusion vessel diameter. For a normal human heart the resulting perfusion flow is about 20 mL/min/100 g to about 200 mL/min/100 g. Using such a system, the seeded cells can achieve a greater seeding concentration of about 5× up to about 1000× greater than achieved under 2D cell culture conditions and, unlike a 2D culture system, the ECM environment allows for the further functional differentiation of cells, e.g., differentiation of progenitor cells into cells that demonstrate organ- or tissue-specific phenotypes having sustained function.

In some aspects, perfusion decellularization comprises cannulating an organ or portion thereof. In some aspects, at least one cannulation is introduced to an organ or portion thereof. In some aspects, at least two cannulations are introduced to an organ or portion thereof. In some aspects, from about 1, 2, 3, 4, 5, 6, 7, 8, 9, or up to 10 cannulations are introduced to an organ or portion thereof. In some cases, a cannula can be a part of a cannulation system. A cannulation system can comprise a size-appropriate hollow tubing for introducing into a vessel, duct, cavity, or any combination thereof of an organ or tissue. Typically, at least one vessel, duct, and/or cavity is cannulated in an organ. A perfusion apparatus or cannulation system can include a holding container for solutions (e.g., a cellular disruption medium) and a mechanism for moving the liquid through the organ (e.g., a pump, air pressure, gravity) via the one or more cannulae. The sterility of an organ or tissue during decellularization and/or recellularization can be maintained using a variety of techniques known in the art such as controlling and filtering the air flow and/or perfusing with, for example, antibiotics, anti-fungals or other anti-microbials to prevent the growth of unwanted microorganisms. In some aspects, a system as described herein can possess the ability to monitor certain perfusion characteristics (e.g., pressure, volume, flow pattern, temperature, gases, pH), mechanical forces (e.g., ventricular wall motion and stress), and electrical stimulation (e.g., pacing). In some aspects, a vascular bed can change over the course of decellularization and recellularization (e.g., vascular resistance, volume), a pressure-regulated perfusion apparatus or cannulation system can be advantageous to avoid or reduce fluctuations. The effectiveness of perfusion can be evaluated in the effluent and in tissue sections. Perfusion volume, flow pattern, temperature, partial O₂ and CO₂ pressures and pH can be monitored using standard methods. In some aspects, sensors can be used to monitor the system (e.g., bioreactor) and/or the organ or tissue. Sonomicromentry, micromanometry, and/or conductance measurements can be used to acquire pressure-volume or preload recruitable stroke work information relative to myocardial wall motion and performance. For example, sensors can be used to monitor the pressure of a liquid moving through a cannulated organ or tissue; the ambient temperature in the system and/or the temperature of the organ or tissue; the pH and/or the rate of flow of a liquid moving through the cannulated organ or tissue; and/or the biological activity of a recellularizing organ or tissue. In addition to having sensors for monitoring such features, a system for decellularizing and/or recellularizing an organ or tissue also can include means for maintaining or adjusting such features. Means for maintaining or adjusting such features can include components such as a thermometer, a thermostat, electrodes, pressure sensors, overflow valves, valves for changing the rate of flow of a liquid, valves for opening and closing fluid connections to solutions used for changing the pH of a solution, a balloon, an external pacemaker, and/or a compliance chamber. To help ensure stable conditions (e.g., temperature), the chambers, reservoirs, and tubings can be water-jacketed.

In some aspects, a method of decellularization comprises providing an organ or portion thereof, cannulating the organ or portion thereof, and perfusing the cannulated organ or portion thereof with a solution or medium via the cannulation. In some aspects, the cannulation occurs at a cavity, vessel, duct, or combination thereof. In some aspects, from about 1 to 3, from about 1 to 5, from about 2 to 3, from about 2 to 5, from about 1 to 8 solutions can be utilized for organ perfusion. In some aspects, a solution is perfused at least two times. In some aspects, a solution is perfused at least 3, 4, 5, 6, 7, 8, 9, or up to 10 times through the organ or portion thereof. Various solutions and mediums can be employed during recelluarization. In some aspects, a solution can be selected from the group comprising: cellular disruption solutions, washing solutions, disinfecting solutions, or combinations thereof.

In some aspects, a cellular disruption solutions is a solutions that can comprise at least one detergent, Table 1. A detergent can be an amphipathic molecule, that can contain both a nonpolar “tail” having aliphatic or aromatic character and a polar “head”. Ionic character of the polar head group can form the basis for broad classification of detergents; they may be ionic (charged, either anionic or cationic), nonionic (uncharged), or zwitterionic (having both positively and negatively charged groups but with a net charge of zero). In some aspects, detergents can be denaturing or non-denaturing with respect to protein structure. Denaturing detergents can be anionic such as sodium dodecyl sulfate (SDS) or cationic such as ethyl trimethyl ammonium bromide (ETMAB). These detergents totally disrupt membranes and denature proteins by breaking protein-protein interactions. Non-denaturing detergents can be divided into nonionic detergents such as Triton X-100, NP40, Tween, bile salts such as cholate, and zwitterionic detergents such as CHAPS.

TABLE 1
Detergents that can be utilized in cellular disruption solutions

| | | Agg. # | | | | | | |
|---|---|---|---|---|---|---|---|---|
| | | (number | | | | | | |
| | | of | | | | | | |
| | | molecules | MW | | CMC | | Cloud | |
| | | per | mono | | mM | | point | |
| Detergent | Type | micelle) | (micelle) | | (% w/v) | | ° C. | Dialyzable |
| Triton X-100 | Nonionic | 140 | 647 | (90K) | 0.24 | (0.0155) | 64 | No |
| Triton X-114 | Nonionic | — | 537 | (—) | 0.21 | (0.0113) | 23 | No |
| NP-40 | Nonionic | 149 | 617 | (90K) | 0.29 | (0.0179) | 80 | No |
| Brij-35 | Nonionic | 40 | 1225 | (49K) | 0.09 | (0.0110) | >100 | No |
| Brij-58 | Nonionic | 70 | 1120 | (82K) | 0.08 | (0.0086) | >100 | No |
| Tween 20 | Nonionic | — | 1228 | (—) | 0.06 | (0.0074) | 95 | No |
| Tween 80 | Nonionic | 60 | 1310 | (76K) | 0.01 | (0.0016) | — | No |
| Octyl | Nonionic | 27 | 292 | (8K) | 23-24 | (~0.70) | >100 | Yes |
| glucoside | | | | | | | | |
| Octyl | Nonionic | — | 308 | (—) | 9 | (0.2772) | >100 | Yes |
| thioglucoside | | | | | | | | |
| SDS | Anionic | 62 | 288 | (18K) | 6-8 | (0.17-0.23) | >100 | No |
| CHAPS | Zwitterionic | 10 | 615 | (6K) | 8-10 | (0.5-0.6) | >100 | Yes |
| CHAPSO | Zwitterionic | 11 | 631 | (7K) | 8-10 | (~0.505) | 90 | Yes |

In some aspects, a washing solution may be utilized during decellularization. A washing solution may be utilized to remove residual solutions such as cellular disruption solutions from an organ or portion thereof as well as residual cellular components, enzymes, or combinations thereof. Suitable washing solutions may comprise water, filtered water, Phosphate buffered saline (PBS), and combinations thereof. PBS can maintain a constant pH and the osmolarity of cells. The pH of most biological materials falls from about 7 to about 7.6. Any concentration of PBS may be utilized as a washing solutions, PBS at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, 55%, 60%, 65%, 70%, 75%, 80%, 85%, 90%, 95%, or up to about 100%. In some aspects, a washing solution may be supplemented with agents. An agent can be an antibiotic, DNaseI, a disinfectant, and the like.

In some aspects, a disinfecting solution may be utilized during decellularization. A disinfecting solution may comprise any number of agents such as antibiotics, disinfectants, or combinations thereof. In some aspects, an antibiotic that can be used in a decellularization solution can be selected from the group comprising: actinomycin, ampicillin, carbenicillin, cefotaxime, fosmidomycin, gentamicin, kanamycin, neomycin, amphotericin, penicillin, polymyxin, streptomycin, broad selection antibiotic, and combinations thereof. Any concentration of antibiotic may be introduced in a disinfecting solution. Suitable concentrations of antibiotics can be: 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, or up to about 60%. Suitable concentrations of antibiotics can be: 0.5 U/ml, 1 U/ml, 5 U/ml, 10 U/ml, 20 U/ml, 30 U/ml, 40 U/ml, 50 U/ml, 60 U/ml, 70 U/ml, 80 U/ml, 90 U/ml, 100 U/ml, 110 U/ml, 120 U/ml, 130 U/ml, 140 U/ml, 150 U/ml, 160 U/ml, 170 U/ml, 180 U/ml, 190 U/ml, 200 U/ml, 300 U/ml, 400 U/ml, 500 U/ml, 600 U/ml, 700 U/ml, 800 U/ml, 900 U/ml, 1000 U/ml, and up to about 1500 U/ml. Suitable concentrations of antibiotics can be: 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 5.5 μg/ml, 6 μg/ml, 6.5 μg/ml, 7 μg/ml, 7.5 μg/ml, 8 μg/ml, 8.5 μg/ml, 9 μg/ml, 9.5 μg/ml, 10 μg/ml, 15 μg/ml, 20 μg/ml, 25 μg/ml, 30 μg/ml, 35 μg/ml, 40 μg/ml, 45 μg/ml, 50 μg/ml, or up to about 60 μg/ml. In some aspects, an antibiotic may be 1% benzalkonium chloride, 100 U/ml penicillin-G, 100 U/ml streptomycin, and 0.25 μg/ml Amphotericin B.

Generally, moderate concentrations of mild (i.e., nonionic) detergents can compromise the integrity of cell membranes, thereby facilitating lysis of cells and extraction of soluble protein, often in native form. Using certain buffer conditions, various detergents effectively penetrate between the membrane bilayers at concentrations sufficient to form mixed micelles with isolated phospholipids and membrane proteins. In some aspects, denaturing detergents such as SDS can bind to both membrane (hydrophobic) and non-membrane (water-soluble, hydrophilic) proteins at concentrations below the CMC (i.e., as monomers). The reaction is equilibrium driven until saturated. Therefore, the free concentration of monomers determines the detergent concentration. SDS binding is cooperative (i.e., the binding of one molecule of SDS increases the probability that another molecule of SDS will bind to that protein) and alters most proteins into rigid rods whose length is proportional to molecular weight. In some aspects, non-denaturing detergents such as Triton X-100 have rigid and bulky nonpolar heads that do not penetrate into water-soluble proteins; consequently, they generally do not disrupt native interactions and structures of water-soluble proteins and do not have cooperative binding properties. The main effect of non-denaturing detergents is to associate with hydrophobic parts of membrane proteins, thereby conferring miscibility to them.

In some aspects, a system for generating an organ or portion thereof or tissue may be controlled by a computer-readable storage medium in combination with a programmable processor (e.g., a computer-readable storage medium as used herein has instructions stored thereon for causing a programmable processor to perform particular steps). For example, such a storage medium, in combination with a programmable processor, may receive and process information from one or more of the sensors. Such a storage medium in conjunction with a programmable processor also can transmit information and instructions back to the bioreactor and/or the organ or tissue. In some aspects, an organ or tissue undergoing recellularization may be monitored for biological activity. Biological activity can be that of the organ or portion thereof or tissue itself such as for cardiac tissue, electrical activity, mechanical activity, mechanical pressure, contractility, and/or wall stress of the organ or tissue. In addition, the biological activity of cells attached or engrafted on to the organ or portion thereof or tissue may be monitored, for example, for ion transport/exchange activity, cell division, and/or cell viability. In some aspects, it may be useful to simulate an active load on an organ or portion thereof during recellularization. In some aspects, a computer-readable storage medium of the invention, in combination with a programmable processor, may be used to coordinate the components necessary to monitor and maintain an active load on an organ or tissue. In some cases, the weight of an organ or portion thereof or tissue may be entered into a computer-readable storage medium as described herein, which, in combination with a programmable processor, can calculate exposure times and perfusion pressures for that particular organ or tissue. Such a storage medium may record preload and afterload (the pressure before and after perfusion, respectively) and the rate of flow. In this embodiment, for example, a computer-readable storage medium in combination with a programmable processor can adjust the perfusion pressure, the direction of perfusion, and/or the type of perfusion solution via one or more pumps and/or valve controls.

In some aspects, perfusion decellularization of an organ or portion thereof can be from about 5%, 10%, 20%, 30%, 40%, 50%, 60%, 70%, 80%, 90%, 95%, or up to about 100% more effective as compared to a non-perfusion based decellularization system. Decellularization of an organ or portion thereof can be determined using various means. In some aspects, decellularization can be determined by histological examination. Histological examination can demonstrate the lack or reduction of cellular material, nuclei, and combinations thereof within a decellularized organ or portion thereof with preservation of the overall structure such as lobules and central veins. In some aspects, decellularization may be determined by immunohistochemical staining. Immunohistochemical staining can demonstrate paucity of cellular factors such as galactosyl-alpha (1,3) galactose (alpha-Gal) following perfusion decellularization. In some aspects, decellularization can be determined using DNA quantification. DNA quantification can comprise assays such as Picogreen. DNA quantification assays can determine an amount of a reduction of DNA in an organ or portion thereof.

A perfusion-based decellularized organ or portion thereof preserves a native scaffold containing the appropriate microenvironment required for the introduction of organ-specific cells, along with an intact vascular network to reconnect to a subject's blood supply and an outer capsule capable of maintaining physiologic pressures. These components are critical for the later use of perfusion recellularization, which also uses perfusion to repopulate vascular and organ-specific regenerative cells onto the organ, where they migrate to the appropriate microenvironment (via the relevant signaling protein markers that remain within the perfusion decellularized scaffold) as the organs are grown and matured in bioreactors under normal physiologic conditions. The resulting organs then can be transplanted utilizing the same techniques as current organ transplantation. Scaffolds created by perfusion decellularization are capable of receiving and incorporating a variety of cells on the organ scaffold utilized.

Immersion Decellularization

In some aspects, immersion-based decellularization of an organ or portion thereof can be performed. In some aspects, whole organs or portions thereof can be decellularized by removing the entire cellular and tissue content from the organ. In some aspects, decellularization can comprise a series of sequential extractions. In some aspects, a first step can involve removal of cellular debris and solubilization of a cell membrane. This can be followed by solubilization of the nuclear cytoplasmic components and the nuclear components. In some aspects, an organ can be decellularized by removing the cell membrane and cellular debris surrounding the organ using gentle mechanical disruption methods. The gentle mechanical disruption methods can disrupt the cellular membrane. However, the process of decellularization should avoid damage or disturbance of the biostructure's complex infra-structure. Gentle mechanical disruption methods can include scraping the surface of the organ, agitating the organ, or stirring the organ in a suitable volume of fluid, e.g., distilled water. In some aspects, the gentle mechanical disruption method can include magnetically stirring (e.g., using a magnetic stir bar and a magnetic plate) the organ or portion thereof in a suitable volume of distilled water until the cell membrane is disrupted and the cellular debris has been removed from the organ or portion thereof. After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of nonionic detergents or surfactants include, but are not limited to, the Triton series, available from Rohm and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl β-D-glucopuranoside, n-heptyl β-D glucopyranoside, n-Octylα-D-glucopyranoside and Nonidet P-40.

Physical Treatments

In some cases, physical treatment of an organ or portion thereof can be done to achieve decellularization. Physical treatment can be used to lyse, kill, and remove cells from an ECM or portion thereof. Physical treatment can utilize temperature, force, pressure, and electrical disruption. In some cases, temperature methods can be used in a rapid freeze-thaw mechanism. For example, by freezing a tissue, microscopic ice crystals can form around the plasma membrane and the cell can be lysed. After lysing the cells, the tissue can be further exposed to liquidized chemicals that can degrade and wash out any residual or undesirable components. In some cases, temperature methods can conserve the physical structure of the ECM scaffold. An organ or portion thereof, and a tissue can be decellularized at a suitable temperature. A suitable temperature can be from about 4° C., 8° C., 10° C., 12° C., 14° C., 16° C., 18° C., 20° C., 22° C., 24° C., 26° C., 28° C., 30° C., 32° C., 34° C., 36° C., 38° C., 40° C., 45° C., 50° C., 55° C., 60° C., or up to about 70° C. A physical treatment can also include the use of pressure. Pressure decellularization can involve the controlled use of hydrostatic pressure applied to a tissue, organ, or portion thereof. Pressure decellularization can be performed at high temperatures in some cases to avoid unmonitored ice crystal formation. In some cases, Electrical disruption of an organ or portion thereof can be performed. Electrical disruption can be done to lyse cells housed in a tissue or organ. By exposing a tissue, organ, or portion thereof to electrical pulses, micropores can be formed at the plasma membrane. The cells can die after their homeostatic electrical balance is ruined through the applied stimulus. This electrical process is documented as Non-thermal irreversible electroporation (NTIRE).

Chemical and Enzymatic Treatments

In some cases, chemical treatment of an organ or portion thereof can be performed to achieve decellularization. Chemicals and/or salts thereof for use in a chemical treatment can be selected for decellularization depending on the thickness, extracellular matrix composition, and intended use of the tissue or organ. For example, enzymes would not be used on a collagenous tissue because they disrupt the connective tissue fibers. However, when collagen is not present in a high concentration or needed in the tissue, enzymes can be a viable option for decellularization. The chemicals and/or salts thereof can be used to kill and remove cells can be but are not limited to acids, alkaline treatments, ionic detergents, non-ionic detergents, and zwitterionic detergents. In some cases, one or more chemicals can comprise a cellular disruption media. A cellular disruption medium can comprise at least one detergent such as Sodium dodecyl sulfate (SDS), polyethyleneglycol (PEG), or Triton X. Detergents can act effectively to lyse the cell membrane and expose the contents to further degradation. For example, after SDS lyses a cellular membrane, endonucleases and/or exonucleases can degrade the genetic contents, while other components of the cell can be solubilized and washed out of the matrix. In some cases, a detergent can be mixed with an alkaline and/or acid treatments due to their ability to degrade nucleic acids and solubilize cytoplasmic inclusions.

One or more cellular disruption media can be used to decellularize an organ or tissue. A cellular disruption medium can comprise at least one detergent such as SDS, PEG, or Triton X. A cellular disruption medium can comprise water such that the medium is osmotically incompatible with the cells. Alternatively, a cellular disruption medium can comprise a buffer (e.g., PBS) for osmotic compatibility with the cells. Cellular disruption media also can include enzymes such as, without limitation, one or more collagenases, one or more dispases, one or more DNases, one or more proteases, and any combination thereof. In some instances, cellular disruption media also or alternatively can include inhibitors of one or more enzymes (e.g., protease inhibitors, nuclease inhibitors, and/or collegenase inhibitors). A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collegenase inhibitors). In some cases, a non-ionic detergent such as Triton X-100 can be utilized. Triton X-100 can disrupt the interactions between lipids and between lipids and proteins. In some cases, Triton X-100 may not disrupt protein-protein interactions, which can be beneficial to keeping the ECM intact. In some cases, EDTA can be utilized. EDTA can be a chelating agent that binds calcium, which can be a component for proteins to interact with one another. By making calcium unavailable, EDTA can prevent the integral proteins between cells from binding to one another. EDTA can be used with trypsin, an enzyme that acts as a protease to cleave the already existing bonds between integral proteins of neighboring cells within a tissue.

A detergent can be administered from about 10 min, 30 min, 60 min, 1 hr., 2 hrs., 3 hrs., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs.

Depending upon the size and/or weight of an organ or portion thereof a chemical treatment such as a detergent can be contacted with the organ or portion thereof from about 2 hours, 3 hours, 4 hours, 5 hours, 6 hours, 7 hours, 8 hours, 9 hours, 10 hours, 11 hours, 12 hours, 13 hours, 14 hours, 15 hours, to about 20 hours per gram of solid organ or tissue with cellular disruption medium.

Including washes, an organ may be perfused for up to about 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some cases, an organ or portion thereof can be perfused from about 12 hours to about 72 hours per gram of tissue. In some aspects, perfusion can be adjusted to physiologic conditions including pulsatile flow, rate, pressure, and any combination thereof.

In some cases, an organ, portion thereof, or tissue can be contacted sequentially with at least two different cellular disruption media. For example, the first cellular disruption medium can include an anionic detergent such as SDS and the second cellular disruption medium can include an ionic detergent such as Triton X. Following contacting, such as perfusion, with at least one cellular disruption medium, a cannulated organ or tissue can be perfused, for example, with wash solutions and/or solutions containing one or more enzymes such as those provided herein. In some cases, alternating the direction of perfusion (e.g., antegrade and retrograde) can help to effectively decellularize an organ, portion thereof, or tissue. Decellularization as provided herein can decellularize an organ or portion thereof from the inside out, resulting in very little damage to the ECM.

In some cases, a sequential method of decellularization can comprise contacting the organ or portion thereof with a cellular disruption media, such as an SDS detergent, followed by a washing step, followed by the addition of one or more chemicals, followed by contacting with a detergent, and ending with at least one wash step. A sequential method of decellularization can comprise at least 1, 2, 3, 4, 5, 6, 7, 8, 9, 10, 11, 12, 13, 14, or up to 15 contacting steps with any media or solution provided herein.

A buffer provided herein can be at a concentration from about 0.1%, 0.5%, 1%, 1.5%, 2%, 2.5%, 3%, 3.5%, 4%, 4.5%, 5%, 10%, 20%, 30%, 40%, 50%, 60%, 70%, 80%, 90%, 95%, or up to about 100%.

### IV. Organ Recellularization

Decellularized organs and portions thereof provided herein can be recellularized. An organ or tissue can be generated by contacting a decellularized organ or tissue as described herein with a population of cells. In some aspects, a population of cells can comprise a regenerative cell. Regenerative cells as used herein are any cells used to recellularize a decellularized organ or tissue. Regenerative cells can be totipotent cells, pluripotent cells, or multipotent cells, and can be uncommitted or committed. Regenerative cells also can be single-lineage cells. In addition, regenerative cells can be undifferentiated cells, partially differentiated cells, or fully differentiated cells. Regenerative cells as used herein include embryonic stem cells (as defined by the National Institute of Health (NIH); see, for example, the Glossary at stemcells.nih.gov on the World Wide Web). Regenerative cells also include progenitor cells, precursor cells, and “adult”-derived stem cells including umbilical cord cells and fetal stem cells. Examples of regenerative cells that can be used to recellularize an organ or portion thereof provided herein can be, without limitation, embryonic stem cells, umbilical cord blood cells, tissue-derived stem or progenitor cells, bone marrow-derived stem or progenitor cells, blood-derived stem or progenitor cells, adipose tissue-derived stem or progenitor cells, mesenchymal stem cells (MSC), skeletal muscle-derived cells, induced pluripotent stem cells (iPSCs), genetically modified cells removing immunogenic factors including but not limited to HLA, or multipotent adult progenitor cells (MAPC). Additional regenerative cells that can be used include tissue-specific stem cells including cardiac stem cells (CSC), multipotent adult cardiac-derived stem cells, cardiac fibroblasts, cardiac microvasculature endothelial cells, or aortic endothelial cells. Bone marrow-derived stem cells such as bone marrow mononuclear cells (BM-MNC), endothelial or vascular stem or progenitor cells, and peripheral blood-derived stem cells such as endothelial progenitor cells (EPC) also can be used as regenerative cells. In some aspects, the number of regenerative cells that can be introduced into a decellularized organ or portion thereof in order to generate an organ or tissue can be dependent on both the organ (e.g., which organ, the size and weight of the organ) or tissue and the type and developmental stage of the regenerative cells. Different types of cells may have different tendencies as to the population density those cells will reach. Similarly, different organ or tissues may be recellularized at different densities. By way of example, a decellularized organ or tissue can be “seeded” with at least about 1,000 (e.g., at least 10,000, 100,000, 1,000,000, 10,000,000, or 100,000,000) regenerative cells; or can have from about 1,000 cells/mg tissue (wet weight, i.e., prior to decellularization) to about 10,000,000 cells/mg tissue (wet weight) attached thereto. In some aspects, regenerative cells can be introduced (“seeded”) into a decellularized organ or tissue by injection into one or more locations.

The methods of recellularizing a tissue or organ matrix as described herein also include re-endothelialization of the tissue or organ matrix with endothelial cells or endothelial progenitor cells. In one embodiment, endothelial cells and endothelial progenitor cells are obtained by culturing embryonic stem cells (ESCs) or induced pluripotent stem cells (iPSCs) under appropriate conditions to direct the stem cells down an endothelial lineage. Endothelial progenitor cells are cells that have begun to differentiate into endothelial cells or have the potential to (e.g., multi-potent; e.g., lineage-restricted; e.g., cells that are destined to become endothelial cells) but are not considered fully differentiated endothelial cells. For example, endothelial cells typically express platelet endothelial cell-adhesion molecule-1 (PECAM1; aka CD31) and may also express one or more of the following markers: VEGFR-1 (aka Flt-1), VEGFR-2 (aka Flk-1), guanylate-binding protein-1 (GBP-1), thrombomodulin (aka CD141), VE-cadherin (aka CD144), von Willebrand factor (vWF), and intercellular adhesion molecule 2 (ICAM-2). Generally, endothelial progenitor cells also are able to take up acetylated LDL, and, further, may migrate toward VEGF and/or form tubes on a Matrigel.

ESCs or iPSCs can be further cultured under conditions that result in fully differentiated endothelial cells. Additionally or alternatively, endothelial cells can be obtained from any number of sources such as blood, skin, liver, heart, lung, retina, and any other tissue or organ that harbors endothelial cells. For example, representative endothelial cells include, without limitation, blood endothelial cells, bone marrow endothelial cells, circulating endothelial cells, human aorta endothelial cells, human brain microvascular endothelial cells, human dermal microvascular endothelial cells, human intestinal microvascular endothelial cells, human lung microvascular endothelial cells, human microvascular endothelial cells, hepatic sinusoidal endothelial cells, human saphenous vein endothelial cells, human umbilical vein endothelial cells, lymphatic endothelial cells, microvessel endothelial cells, microvascular endothelial cells, pulmonary artery endothelial cells, retinal capillary endothelial cells, retinal microvascular endothelial cells, vascular endothelial cells, umbilical cord blood endothelial cells, and combinations thereof. As those of skill in the art would understand, this is not intended to be an exhaustive list of endothelial cells.

Endothelial cells can be obtained, for example, from one of the many depositories of biological material around the world. See, for example, the American Type Culture Collection (ATCC.org on the World Wide Web) or the International Depositary Authority of Canada (IDAC; nml-lnm.gc.ca on the World Wide Web). Endothelial cells or endothelial progenitor cells also can be obtained from the individual that will be the recipient of the transplanted tissue or organ matrix. These cells would be considered to be autologous to the recipient. Additionally, under certain circumstances, the relationship between the tissue or organ matrix and the endothelial cells or endothelial progenitor cells can be allogeneic (i.e., different individuals from the same species); in other instances, the relationship between the tissue or organ matrix and the endothelial cells or endothelial progenitor cells can be xenogeneic (i.e., individuals from different species).

A composition that includes endothelial cells or endothelial progenitor cells typically is delivered to a tissue or organ matrix in a solution that is compatible with the cells (e.g., in a physiological composition) under physiological conditions (e.g., 37° C.) and under non-physiologic conditions (e.g. 4-35° C.)). A physiological composition, as referred to herein, can include, without limitation, buffers, nutrients (e.g., sugars, carbohydrates), enzymes, expansion and/or differentiation medium, cytokines, antibodies, repressors, growth factors, salt solutions, or serum-derived proteins. As used herein, a composition that “consists essentially of” endothelial cells or endothelial progenitor cells is a composition that is substantially free of cells other than endothelial cells or endothelial progenitor cells but may still include any of the components that might be found in a physiological composition (e.g., buffers, nutrients, etc.).

To optimize re-endothelialization, endothelial cells or endothelial progenitor cells generally are introduced into an organ or tissue matrix by perfusion. As with the pre-cellular perfusion, and as described in WO 2007/025233, perfusion occurs via the vasculature or vasculature-type structure of the organ or tissue matrix. Perfusion to re-endothelialize an organ or tissue matrix should be at a flow rate that is sufficient to circulate the physiological composition of cells through the vasculature. Perfusion with the endothelial cells or endothelial progenitor cells can be multi-directional (e.g., antegrade and retrograde) to even further optimize re-endothelialization. Perfusion of cells may be followed by a static hold time to enhance engraftment prior to reperfusion of the organ or tissue matrix.

In some aspects, at least one type of cell (i.e., a cocktail of cells) can be introduced into a decellularized organ or portion thereof. For example, a cocktail of cells or a population of cells can be injected at multiple positions in a decellularized organ or tissue or different cell types can be injected into different portions of a decellularized organ or portion thereof. Alternatively, or in addition to injection, regenerative cells, a population of cells, or a cocktail of cells can be introduced by perfusion into a cannulated decellularized organ or portion thereof. For example, regenerative cells can be perfused into a decellularized organ using a perfusion medium, which can then be changed to an expansion and/or differentiation medium to induce growth and/or differentiation of the regenerative cells. During recellularization, an organ or tissue can be maintained under conditions in which at least some of the regenerative cells can proliferate, multiply, differentiate, and any combination thereof in the decellularized organ or portion thereof. In some aspects, those conditions can include, without limitation, the appropriate temperature, pressure, electrical activity, mechanical activity, force, the appropriate amounts of O₂ and/or CO₂, an appropriate amount of humidity, sterile or near-sterile conditions, and any combination thereof. During recellularization, the decellularized organ or tissue and the regenerative cells attached thereto can be maintained in a suitable environment. For example, the regenerative cells may require a nutritional supplement (e.g., nutrients and/or a carbon source such as glucose), exogenous hormones or growth factors, and/or a particular pH.

In some aspects, regenerative cells as provided herein can be allogeneic to a decellularized organ or portion thereof (e.g., a human decellularized organ or tissue seeded with human regenerative cells), or regenerative cells can be xenogeneic to a decellularized organ or portion thereof (e.g., a pig decellularized organ or tissue seeded with human regenerative cells). “Allogeneic” as used herein refers to cells obtained from the same species as that from which the organ or tissue originated (e.g., self (i.e., autologous) or related or unrelated individuals), while “xenogeneic” as used herein refers to cells obtained from a species different than that from which the organ or tissue originated.

In some embodiments, an endothelial cell can be perfused into a decellularized liver. Populations of endothelial cells may engraft onto the decellularized liver matrix as described above. However, the inventors demonstrate in the Examples below the surprising and unexpected result that an endothelial cell upon engraftment may migrate and/or proliferate into more liver cell-like phenotypes. For example, an endothelial cell such as human umbilical endothelial cells (HUVECs) are shown in the examples below to adopt a liver sinusoidal endothelial cell (LSEC)-like phenotype after engraftment. A hallmark feature of LSECs in normal liver tissue is the presence of plasma membrane fenestrations which enable diffusion of nutrients and waste products between the capillary vessels and the adjacent parenchymal space. Accordingly, in some embodiments, a recellularized organ may comprise a fenestrated endothelium that was absent from the decellularized liver, and or absent in the seeded or introduced cell population but may be present following engraftment and/or migration into the decellularized liver matrix.

Such transformation or plasticity can be monitored by determining an expression level of certain genes in a parenchymal or sinusoidal niche of the recellularized organ. In some cases, a gene marker that can be used to determine sinusoidal marker expression which can be but not limited to VEGFR-3, D2-40, STAB2, CD31, RPL19, or LYVE-1. In some cases, who biopsies of the seeded liver graft can be taken and look for the expression of sinusoidal markers including but not limited to VEGFR-3, D2-40, STAB2, CD31, RPL19, or LYVE-1. In some cases, increased expression of sinusoidal genes and or the direct detection of fenestration measured following engraftment and/or migration and/or proliferation into the parenchymal space.

### V. Uses of Organs and Portions Thereof

Decellularized and recellularized organs or portions thereof provided herein can be used in a variety of applications. For example, organs or portions thereof can be implanted into a subject. In some aspects, a composition of the present invention, such as an organ or portion thereof, may be transplanted into a subject that has a disease. Relevant diseases that may require organ transplantation include but are not limited to: organ failure, cardiomyopathy, cirrhosis, chronic obstructive pulmonary disease, pulmonary edema, biliary atresia, emphysema and pulmonary hypertension, coronary heart disease, valvular heart disease, congenital heart disease, coronary artery disease, pancreatitis, cystic fibrosis, diabetes, hepatitis, hypertension, idiopathic pulmonary fibrosis, polycystic kidneys, short gut syndrome, injury, birth defects, genetic diseases, autoimmune disease, and any combination thereof. Implants, according to the invention, can be used to replace or augment existing tissue. For example, to treat a subject with a kidney disorder by replacing the dysfunctional kidney of the subject with an exogenous or engineered kidney. The subject can be monitored after implantation of the exogenous kidney, for amelioration of the kidney disorder. Any decellularized organ or portion thereof provided herein can be utilized for implantation into a subject.

In some aspects, a composition provided herein, such as a solid organ or portion thereof can have from about 1% to about 100% of its native function after decellularization. In some aspects, a composition provided herein, such as a solid organ or portion thereof can have from about 5%, 10%, 20%, 30%, 40%, 50%, 60%, 70%, 80%, 90%, 95%, or up to about 100% of its native function after decellularization.

In some aspects, particular organs or portions thereof may be suitable for transplantation when they function below that of their native counterpart. For example, a liver and a kidney may need approximately from about 20% of the total organ function to provide the needed organ function to save a person from liver failure or remove them from dialysis. In some aspects, a liver and kidney may need approximately from about 20-30%, 30-40%, 20-50%, 20-60%, 40-60% of the total organ function to be suitable for transplantation. In some aspects, an organ may function equal to a native counterpart. For example, a heart is more complicated, in that, it may need from about 80%, 85%, 90%, 95%, 96%, 97%, 98%, 99%, or up to about 100% function at the time of transplantation.

In some aspects, a lifespan of a subject may be extended after transplantation of a composition, such as an organ or portion thereof provided herein. For example, a lifespan of a subject may be extended from about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or up to about 100 years after transplantation. In some aspects, transplantation of a composition, such as an organ or portion thereof provided herein, may reduce the need of a secondary treatment in a subject. Secondary treatments can refer to dialysis, pacemakers, respirators, and combinations thereof.

Decellularized and recellularized organs or portions thereof provided herein can also be used in vitro to screen a wide variety of compounds, for effectiveness and cytotoxicity of pharmaceutical agents, chemical agents, growth/regulatory factors. The cultures can be maintained in vitro and exposed to the compound to be tested. The activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The effect of various drugs on normal cells cultured in the reconstructed artificial organs may be assessed.

Decellularized and recellularized organs or portions thereof provided herein can be used in vitro to filter aqueous solutions, for example, a reconstructed artificial kidney may be used to filter blood. Using the reconstructed kidney provides a system with morphological features that resemble the in vivo kidney products. This system may be suitable for hemodialysis. In some aspects, the system may also be useful for hemofiltration to remove water and low molecular weight solutes from blood. The artificial kidney may be maintained in vitro and exposed to blood which may be infused into the luminal side of the artificial kidney. The processed aqueous solution may be collected from the abluminal side of the engineered kidney. The efficiency of filtration may be assessed by measuring the ion, or metabolic waste content of the filtered and unfiltered blood.

Decellularized and recellularized organs or portions thereof provided herein can be used as a vehicle for introducing genes and gene products in vivo to assist or improve the results of the transplantation and/or for use in gene therapies. For example, cultured cells, such as endothelial cells, can be engineered to express gene products. The cells can be engineered to express gene products transiently and/or under inducible control or as a chimeric fusion protein anchored to the endothelial cells, for example, a chimeric molecule composed of an intracellular and/or transmembrane domain of a receptor or receptor-like molecule, fused to the gene product as the extracellular domain. In another embodiment, the endothelial cells can be genetically engineered to express a gene for which a patient is deficient, or which would exert a therapeutic effect. The genes of interest engineered into the endothelial cells or parenchyma cells need to be related to the disease being treated. For example, for a kidney disorder, the endothelial or cultured kidney cells can be engineered to express gene products that would ameliorate the kidney disorder.

Provided herein are also compositions and methods of generating engineered organs or portions thereof comprising a population of cells. In some aspects, at least two populations of cells can be introduced into a decellularized organ or portion thereof. Organs that can be engineered include, but are not limited to, heart, kidney, liver, pancreas, spleen, urinary bladder, ureter, urethra, skeletal muscle, small and large bowel, esophagus, stomach, brain, spinal cord and bone.

In some cases, a recellularized liver can be transplanted into a recipient. A recellularized liver as described herein to be transplanted as a functional organ. In some cases, function can be determined through patency of the vasculature of the organ for a prolonged period of time. Patency can be measured using, for example, the methods described in the examples below. For instance, a graft can be connected to a peristaltic pump and subjected to physiologically achievable venous pressure. In some cases, a pressure of between 5 to 50 mm Hg can be utilized to determine patency through the venous vasculature or 40-120 mm Hg through the arterial vasculature.

Patency can be assessed over time. In some cases, patency can be assessed for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours. In some cases, patency can be assessed for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In some embodiments, functionality can be assessed by determining consumption of certain metabolites (i.e. glucose, lactate, glutamine, glutamate and ammonia). Such consumption can be determined by perfusing in a continuous line of the metabolite and measuring a rate of consumption of the metabolite over time using, for example, a change in electrochemical potential. Methods and for detection of these metabolites are readily apparent to a skilled artisan, and sensors for determining these metabolites are readily available.

In some embodiments, the rate of consumption of a metabolite can be used to determine successful engraftment of endothelial cells onto a decellularized matrix. For example, the inventors demonstrate in the Examples below that a glucose consumption rate can be correlated to successful endothelialization in a recellularized liver. Furthermore, the Examples below demonstrate the surprising and unexpected result that glucose consumption rate can be correlated to in vivo graft patency, thus enabling a glucose consumption rate to be used a surrogate for in vivo patency. Furthermore, other metabolite consumption such as lactate, glutamine, glutamate and ammonia are expected to also be predictive of in vivo patency.

In some cases, a recellularized liver can be transplanted along with systemic administration of an immunosuppressor. As demonstrated in the Examples below, administration of an immunosuppressor may prolong patency of a transplanted organ. In some cases, an immunosuppressor can be a corticosteroid, a Janus kinase inhibitor, a calcineurin inhibitor, an mTOR inhibitor, an IMDH inhibitor, a biologic, a monoclonal antibody, or any combination thereof. Examples of corticosteroids can include prednisone, budesonide, prednisolone, and methylprednisolone. Examples of Janus kinase inhibitors can include tofacitinib. Examples of calcineurin inhibitors can include cyclosporine and tacrolimus. Examples of mTOR inhibitors can include sirolimus and everolimus. Examples of IMDH inhibitors can include azathioprine, leflunomide, and mycophenolate. Examples of immunosuppressive biologics can include abatacept, adalimumab, anakinra, certolizumab, etanercept, golimumab, infliximab, ixekizumab, natalizumab, rituximab, secukinumab, tocilizumab, ustekinumab, and vedolizumab. Examples of immunosuppressive monoclonal antibodies can include basiliximab and daclizumab. Such immunosuppressors can be administered to a recipient of a recellularized liver via enteral routes (including oral, gastric or duodenal feeding tube, rectal suppository and rectal enema), parenteral routes (injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal or topical (including epicutaneous, dermal, enema, eye drops, ear drops, intranasal, vaginal) administration. Immunosuppressors can be administered to a recipient at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

Other embodiments and used of the invention will be apparent to those skilled in the art from consideration of the specification and practice of the invention disclosed herein. All U.S. Patents and other references noted herein for whatever reason are specifically incorporated by reference. The specification and examples should be considered exemplary only with the true scope and spirit of the invention indicated by the following claims.

### VI. Examples

Example 1 through Example 11 below describe a study showing successful recellularization a decellularized porcine liver. The study demonstrates the ability to produce bioengineered whole liver grafts with functional vasculature that can retain long-term in vivo vascular patency. Adequate endothelization is sufficient to prevent thrombotic events and removes the need for further matrix modification of heparinization, providing a key path forward to engineering a fully functional transplantable liver.

### Example 1—Culturing of HUVEC Cells

Human umbilical vein endothelial cells (HUVECs, Lonza C2517A) were cultured in EGM-2 (Lonza CC-3162) medium with no antibiotics. Cells were cultured in flasks at 37° C. and 5% CO₂ and passaged with 0.25% trypsin at 90-100% confluency. The highest passage used for seeding liver grafts was passage 11. HUVECs were used for all re-endothelialization of decellularized liver grafts.

### Example 2—Decellularization of Rat Liver

For liver isolation, the caval vein was exposed through a median laparotomy, dissected and canulated using a mouse aortic canula (Radnoti Glass, Monrovia, Calif.). The hepatic artery and vein and the bile duct were transsected and the liver was carefully removed from the abdomen and submerged in sterile PBS (Hyclone, Logan, Utah) to minimize pulling force on portal vein. 15 minutes of heparinized PBS perfusion was followed by 2-12 hours of perfusion with 1% SDS (Invitrogen, Carlsbad, Calif.) in deionized water and 15 minutes of 1% Triton-X (Sigma, St. Louis, Mo.) in deionized water. The liver was then continuously perfused with antibiotic containing PBS (100 U/ml penicillin-G (Gibco, Carlsbad, Calif.), 100 U/ml streptomycin (Gibco, Carlsbad, Calif.), 0.25 .mu.g/ml Amphotericin B (Sigma, St. Louis, Mo.)) for 124 hours.

120 minutes of SDS perfusion followed by perfusion with Triton-X 100 were sufficient to generate a completely decellularized liver. Movat pentachrome staining of decellularized liver confirmed retention of characteristic hepatic organization with central vein and portal space containing hepatic artery, bile duct and portal vein.

### Example 3—Decellularization of Porcine Liver

Whole livers were excised from cadaveric pigs. The suprahepatic vena cava, inferior vena cava, portal vein, and bile duct were cannulated and flushed with 150 ml of sterile saline. The cannulated livers were perfusion decellularized with 1× Triton X-100 followed by 0.6% sodium dodecyl sulfate until complete at a perfusion pressure maintained between 8-12 mmHg. The decellularized livers were disinfected with 1000 ppm peracetic acid (PAA). The decellularized grafts were washed with phosphate buffered saline (PBS) and stored. Decellularization and recellularization utilized a custom-built perfusion system to automatically adjust flow to maintain a defined pressure utilizing Cole-Palmer peristatic pumps.

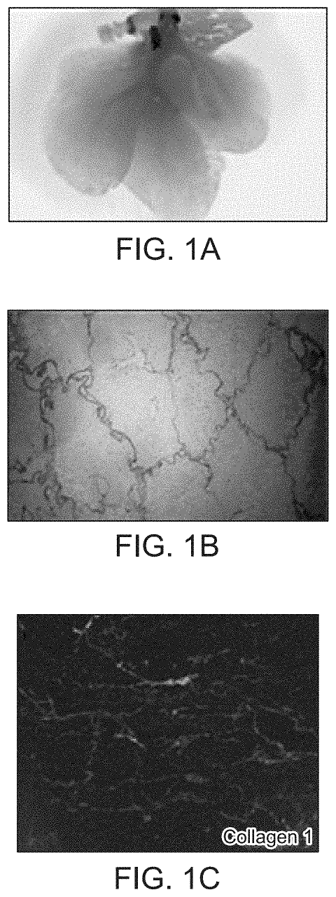

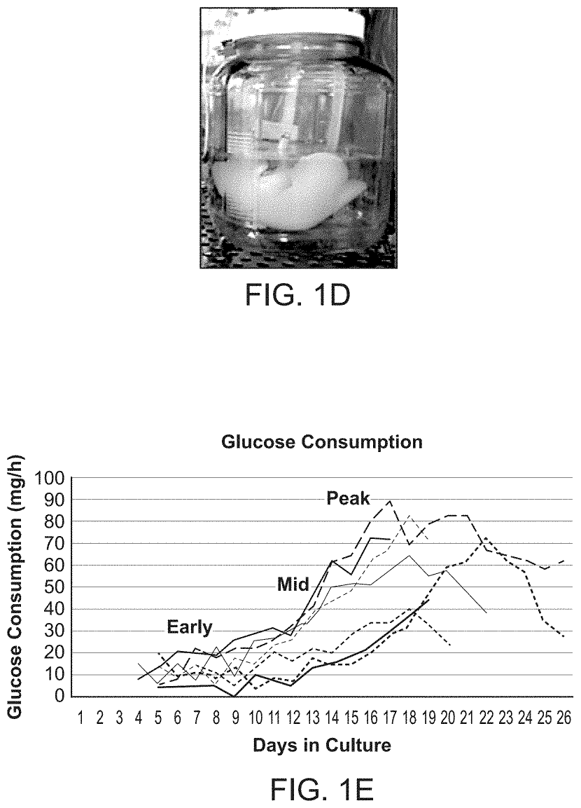

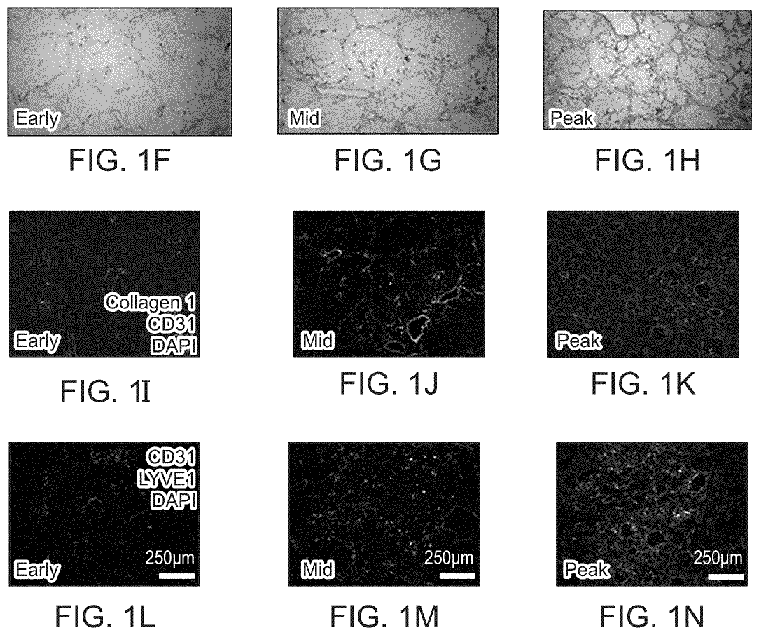

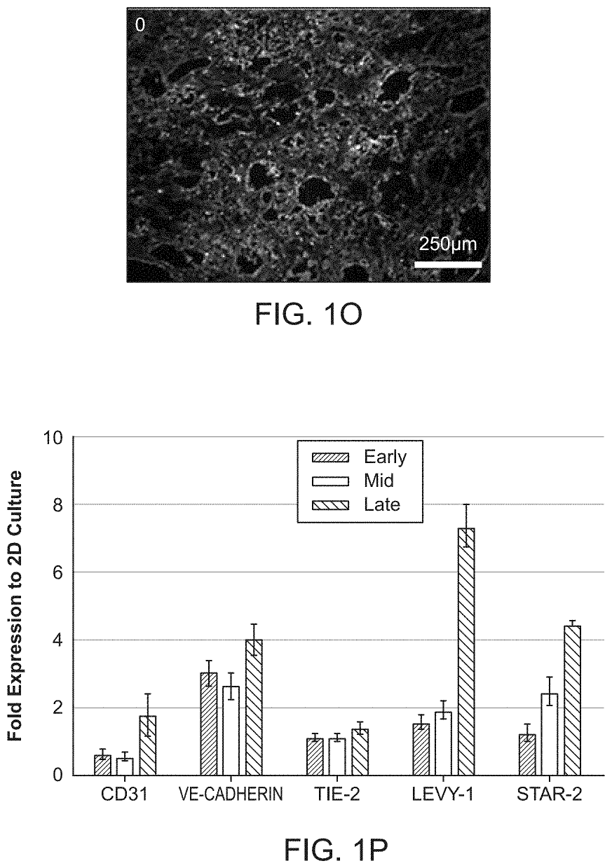

Whole liver decellularization was performed in an ISO 7 cleanroom and optimized to maintain native liver architecture (FIG. 1, A to C) under aseptic conditions, or can be sterilized utilizing irradiation or other common methods of sterilization.

### Example 4—Seeding of HUVEC Cells

The medium used for culturing the HUVECs was also used for seeding and maintaining re-endothelialized liver grafts. Decellularized porcine livers were placed in a custom bioreactor containing 800 ml of media, connected to the inlet via the suprahepatic vena cava and perfused at 12 mmHg with culture medium prior to seeding. HUVECs (1.5×10⁶) were resuspended in 100 ml of media and seeded through the suprahepatic vena cava. The injected cell suspension was left under static conditions for one hour and then perfusion was restarted with the pressure limited to 12 mmHg. After 24 hours, the perfusion inlet was changed from the suprahepatic vena cava to the portal vein and the seeding protocol was repeated. The perfusion rate for the entire duration of culture was limited to 12 mmHg. Re-endothelialized grafts were allowed to culture in a continuous perfusion loop with metabolites (glucose, lactate, glutamine, glutamate and ammonia) being monitored in collected media samples. Bioreactor medium was changed regularly and volume increased depending on the rate of glucose depletion in the circulating medium. Glucose consumption was calculated based on the glucose concentration of fresh medium, added volume and duration until following reading. Glucose consumption can drive media volumes to further control either the acceleration or reduction of overall growth.

Decellularized livers were mounted in custom bioreactors (FIG. 1D), continuously perfused with media at 12 mmHg resulting in a flow from 200-250 ml/min and seeded with human endothelial cells (HUVECs). Metabolic consumption of glucose, glutamine, production of lactate, glutamate, and ammonia were measured daily to define a non-destructive marker for endothelial cell growth. Analysis of glucose consumption (mg/hr) consistently demonstrated a standard curve compared to other analytes with a defined early, middle (mid) rapid growth and peak phase (FIG. 1E).

### Example 5—Endothelial Characterization and Phenotype

Histology

Tissue was perfused with PBS and fixed via perfusion with 10% Neutral Buffered Formalin (VWR 16004-128) and paraffin embedded, sectioned and stained via standard histologic techniques. Immunofluorescence slides were deparaffinized, rehydrated and retrieval was performed in citrate buffer, pH 6.0 (Abcam AB93678) in a Biocare Medical Decloaker (Biocare DC2012). Slides were permeabilized in 0.05% Tween® 20 (Sigma P9416) in PBS and blocked with Sea Block (Thermo 37527). Mouse Anti-CD31 (Abcam AB187377) and Rabbit Anti-Collagen I (Abcam AB34710) were each diluted 1:100 in Sea Block, and Goat Anti-Mouse Alexa Fluor 488 (Thermo A11029) and Goat Anti-Rabbit Alexa Fluor 555 (Thermo A21429) were each similarly diluted 1:500 in Sea Block. Slides were stained with 4′,6-diamidino-2-phenylindole (Thermo D1306) diluted 1:200 in PBS and mounted using ProLong Antifade Mountant (Thermo P36961). Fluorescence slides were imaged on a Zeiss Axioskop 40 and H&E slides were imaged on an Accuscope 3012.

Histopathological analysis of different stages of graft maturation was performed in order to understand the endothelialization process. Three grafts were analyzed in 3 different stages of the glucose consumption curve. Early stage, which is the period after HUVECs seeding but prior to the noted surge in glucose consumption. Middle stage, the period of the glucose consumption rate surge defined by an increase of >10% in glucose consumption for 3 consecutive days. Peak stage, the period where the glucose consumption plateaus or starts to decrease after reaching the peak glucose consumption rate. One graft from each stage was analyzed histologically using hematoxylin & eosin (H&E) staining and immunohistochemistry as well as qPCR for endothelial markers (FIGS. 1A-1P). The rate of glucose consumption was noted as well as the duration of incubation prior to analysis.

Cold Storage/Viability Studies

Grafts were tested using cold storage for 6 hours to mimic the conditions and time required for transportation and implantation of the graft. Re-endothelialized grafts were flushed through the portal vein with 300 ml of 4° C. phosphate buffered saline PBS to remove residual medium. This was repeated with cold organ preservation solution (HTK or UW-Belzer) to preserve the re-endothelialized graft for transport. The grafts were placed in a sterile bowl with cold storage solution and placed on ice for three hours. Grafts were then flushed with 300 ml of room temperature PBS, returned to a bioreactor with fresh culture medium and followed for 48 hours to assess changes in GCR.

RNA Extraction and Quantitative Reverse-Transcriptase PCT (qRT-PCR)

RNA extraction was performed using the Trizol reagent as per manufacturer's instructions (Invitrogen). Last 1 μg of RNA was transcribed to cDNA using the Superscript III First-Strand synthesis (Invitrogen). Gene expression analysis was performed using the Platinum SYBR green qPCR supermix-UDG kit (Invitrogen) in a ViiA 7 Real-Time PCR instrument (Thermo Fisher Scientific, Waltham, Mass.). Ribosomal protein L19 (RPL19) was used as a housekeeping gene for normalization. Primer sequences: RPL19 5′ATTGGTCTCATTGGGGTCTAAC3′, 5′AGTATGCTCAGGCTTCAGAAGA3′, STAB2 5′GCAAGAAGATGTGATAGGAAGTCTC3′, 5′ACAACACCGAGGTTGGAGAT3′, LYVE1 5′TTTGCAGCCTATTGTTACAACTCAT3′, 5′GGGATGCCACCCAGTAGGTA3′ and CD31 5′TCTGCACTG CAGGTATTGACAA, 5′CTGATCGATTCGCAACGGA3′.

Statistical Analysis

Correlations between percentile graft flow and metabolic parameters were statistically compared using binary correlation and linear logistic regression. Surgical model parameters were compared using student t-test. Graft perfusion times in implanted bioengineered livers were compared using student t-test.

Results

Histological examination of the early, mid, and peak glucose consumption rate (PGCR) correlated with endothelial density via H&E (FIG. 1, F to H) leading to glucose consumption being identified as a vital in vitro variable that correlated strongly with the level of reendothelialization. Bioreactor media volumes were adjusted daily to maintain 24 h glucose levels above 250 mg/L or 25% of baseline media concentrations.

Immunostaining of liver grafts at early, mid and peak glucose consumption (PGCR) levels with CD31 and Collagen I (FIG. 1, I to K) further demonstrated localization to vascular structures and uniform overall coverage. Endothelial engraftment was primarily localized to the larger vessels during the early phase (<20 mg/hr.) with notable expansion into the sinusoidal spaces at mid and peak phases. Additional immunostaining with LYVE-1 (FIG. 1, L-N) demonstrated highest expression in the parenchymal niche with little expression in larger vessels. RNA expression of sinusoidal endothelial makers via RT-PCR at peak (Day 14) demonstrated an upregulation of LYVE1 (7.3+/−0.62) and STAB2 (4.5+/−0.05) compared to HUVEC cells in culture and demonstrated a significant increase from early to late phase. CD31 was relatively unchanged (1.8+/−0.64) (FIG. 1 O) consistent with reported sinusoidal endothelial phenotypes [24] compared to cultured HUVEC controls. Immunostaining for LYVE1 and CD31 confirmed the gene expression data, LYVE1 was weakly detected in the early phase and became consecutively stronger in the mid and peak glucose phase (FIG. 1, I to M, P). LYVE1 expression was primarily localized to the sinusoid regions, CD31 expression remained highest in the larger vessels at early and late phases. These results demonstrate the phenotypic plasticity of HUVECs when placed in the liver matrix.

The active measurement of and analysis of metabolic markers provided critical growth parameters. Specifically, PGCR demonstrated the ability to not only be predictive of the level of endothelialization of bioengineered liver grafts while in culture, but it also correlated to in vivo performance. This reflects the importance of appropriate seeding, handling and monitoring of bioengineered grafts during various growth phases including early, mid and peak phases. The resulting functional vascular graft was independent of days in culture, but instead dependent upon metabolic activity. These phases correlated with increased endothelial coverage starting in the larger vessels in migrated into the parenchymal space in mid and peak phases. In addition, LYVE-1 expression and localization was increased in the parenchymal space demonstrating the plasticity of HUVECs and the importance of the ECM. High PGCR grafts resulted in in-vivo vascular perfusion on average of 3.5 days when transplanted into a pig liver transplant model.

### Example 6—Acute Studies

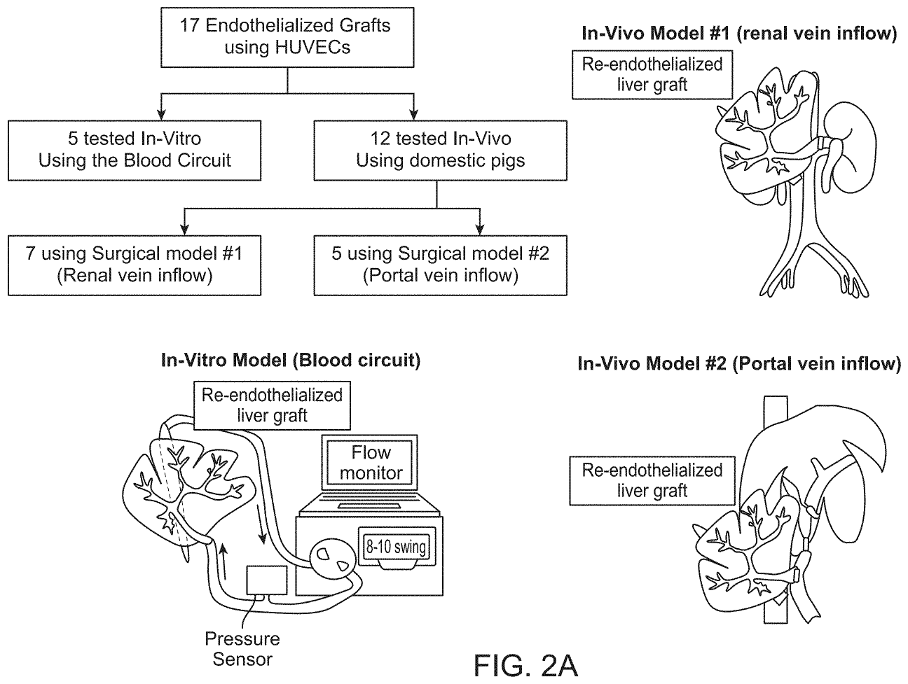

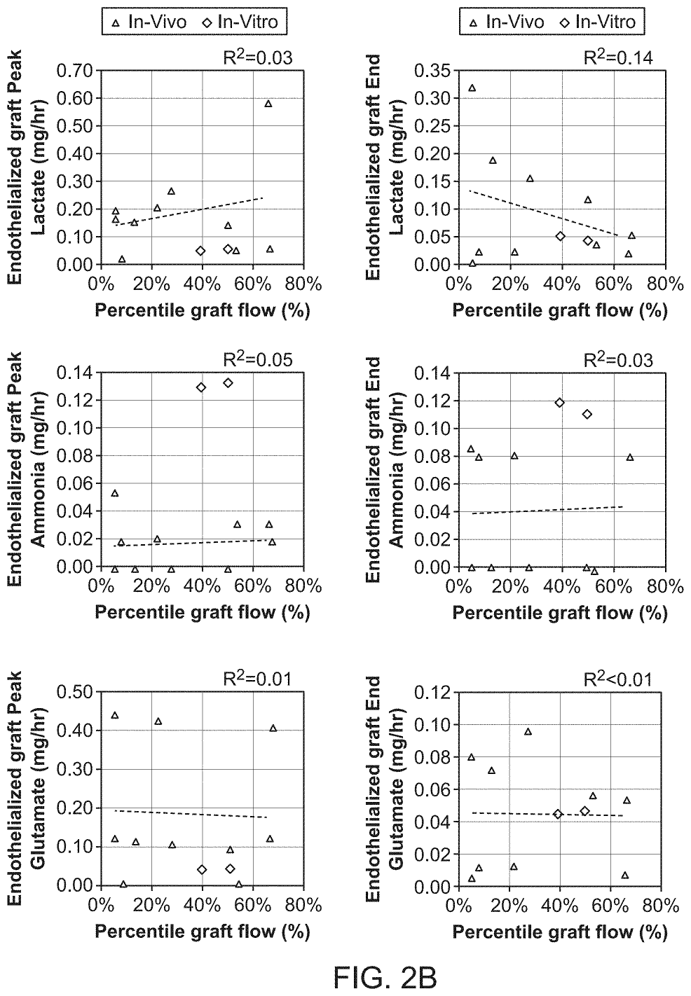

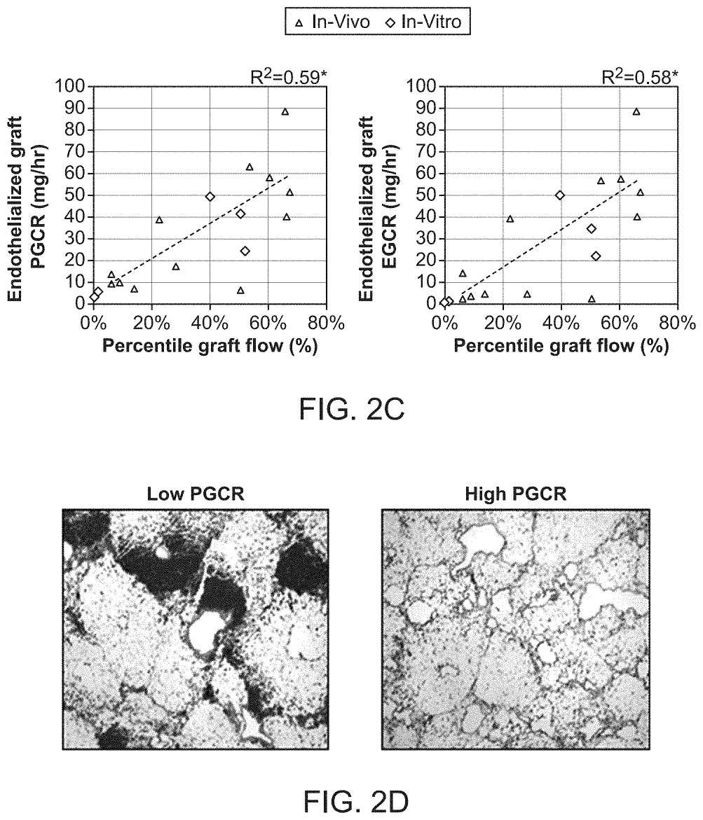

In vitro metabolic consumption profiles were screened as predictors of sustained continuous blood flow through the evaluation of re-endothelialized liver grafts as a percent of blood flow after 30 minutes, compared to baseline flow in their absence, on 17 grafts including 5 in vitro and 12 in vivo (FIG. 2A). The peak glucose and end consumption rates of ammonia, glutamate and lactate were plotted against the graft flow achieved in-vitro and in-vivo (FIG. 2B). While the exemplary study illustrated in FIG. 2B shows that ammonia, glutamate and lactate levels failed to predict subsequent blood flow, subsequent studies may show a correlation. Peak and end glucose consumption correlated strongly with sustained in vitro and in vivo blood flow (r=0.767, r²=0.589, P<0.001, and (r=0.760, r²=577, P<0.001 respectively) (FIG. 2C). Histological evaluation of low glucose consumption grafts following blood loop and saline flushing presented evidence of blood pooling and compaction within the graft, while high glucose consumption grafts confirmed the presence of endothelial cells and clearing of blood from the graft (FIG. 2D). Unseeded control liver grafts consistently resulted in zero blood flow after <5 minutes in both in vitro and in vivo studies.

### Example 7—Surgical Model

Two surgical models were evaluated during the in vivo short-term analysis. The first surgical model was the renal vein surgical model where the left renal vein was utilized for an end-to-end anastomosis with the graft's portal vein and an end-to-side anastomosis between the graft's and pig's intrahepatic vena cavae, a could also be used. The second surgical model was a portal vein surgical model with an end-to-side anastomosis between the graft's and pig's portal veins. Subsequently, all portal branches supplying the pig native liver were tied off preserving the first portal branch supplying the caudate lobe and the right lateral lobe. Additionally, a constricting ribbon was applied surgically around the native portal vein distal to the anastomosis to enhance blood flow to the bioengineered liver graft. This sequence was followed to elevate the portal vein pressure while avoiding native portal vein clamping. Both models were evaluated for their hemodynamic properties including pressure and flow measurements.

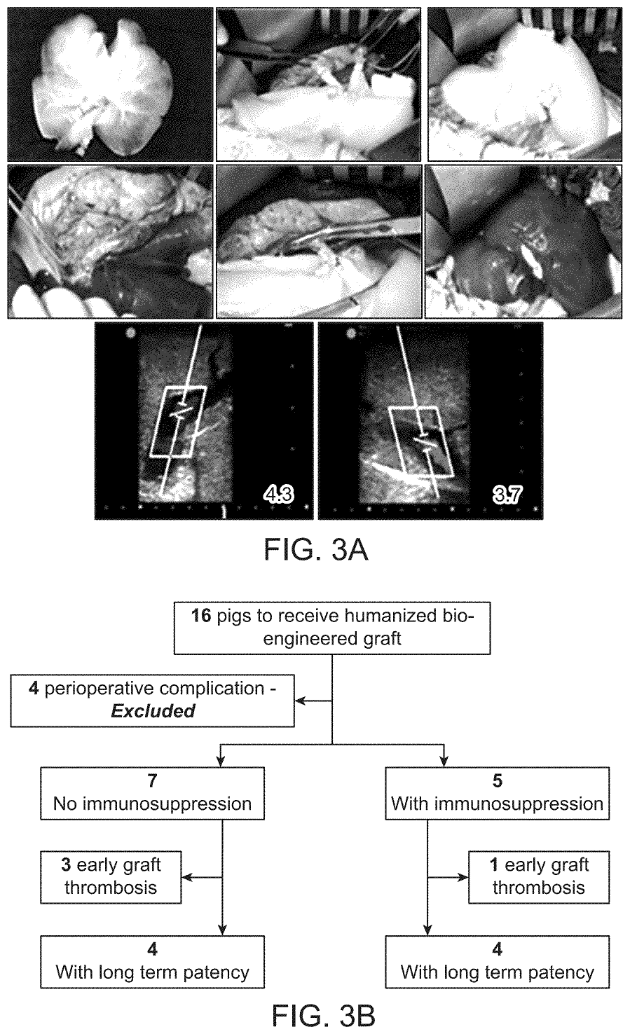

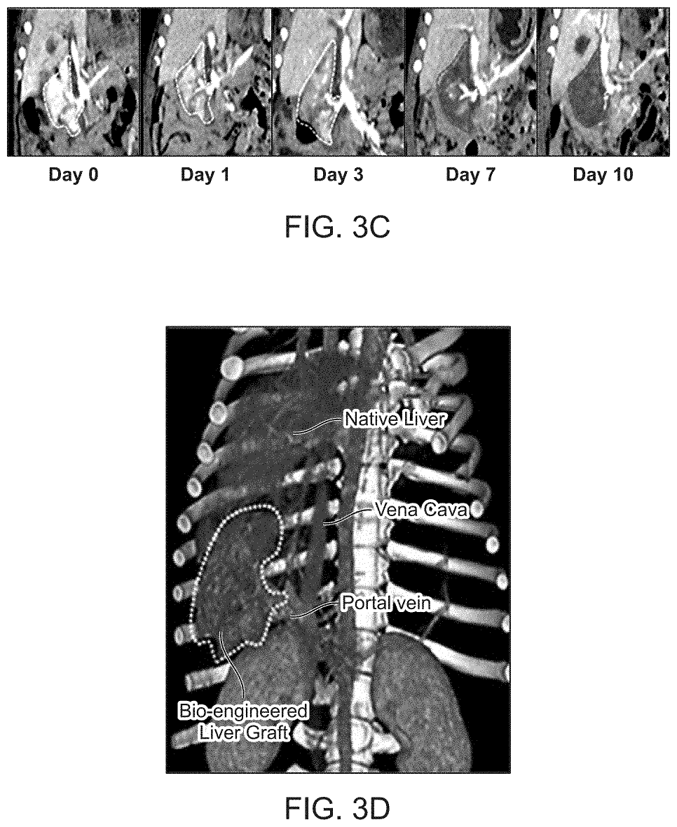

Portal and renal vein anastomosis surgical models were compared for surgical, hemodynamic, and graft variables (Table 2). The portal vein surgical model provided higher venous pressure system (mean of 8.4 mmHg versus 3.43 mmHg, p=0.001) and higher absolute blood flow rates (mean of 105 ml/min versus 30.1 ml/min) when compared to the renal vein surgical model. Based on these results, the portal surgical model was used for all long-term studies (FIG. 3A).

TABLE 2
Comparison of renal versus portal surgical model for liver graft implantation.

| | | | |
|---|---|---|---|
| Variable | Renal Vein Model | Portal Vein Model | Statistical Significance |

Baseline Characteristics of Host and Graft

| | | | |
|---|---|---|---|
| Host variables | | | |
| Pig Weight (Kg) | 31.5 ± 2.74 | 36.6 ± 3 | P = 0.012 |
| MAP baseline (mmHg) | 48.7 ± 9.7 | 59.2 ± 13.8 | P = 0.151 |
| Venous conduit blood flow (ml/min) | 120 ± 37 | 414 ± 16.7 | P < 0.001 |
| Venous conduit pressure (mmHg) | 3.43 ± 1.4 | 8.4 ± 2.2 | P = 0.001 |
| Re-endothelialized graft variables | | | |
| Peak Glucose consumption (mg/hr) | 20.5 ± 14.1 | 54.7 ± 29 | P = 0.022 |
| Incubation time (days) | 13.9 ± 1.6 | 19 ± 4.3 | P = 0.015 |
| Weight (gm) | 147.7 ± 47.2 | 153 ± 50.3 | P = 0.88 |

Results of short-termstudies
Absolute blood flow

| | | | |
|---|---|---|---|
| Initial graft flow (ml/min) | 47.4 ± 25.9 | 106 ± 57 | P = 0.013 |
| Graft flow at 15 minutes (ml/min) | 49 ± 29.8 | 111 ± 36.8 | P = 0.009 |
| Graft flow at 30 minutes (ml/min) | 30.1 ± 25.3 | 105 ± 47.4 | P = 0.005 |

Percentile blood flow

| | | | |
|---|---|---|---|
| Percentile flow initial (%) | 39.1 ± 13.1 | 26 ± 14.4 | P = 0.128 |
| Percentile flow at 15 minutes (%) | 40.3 ± 22.2 | 53.4 ± 16 | P = 0.287 |
| Percentile flow at 30 minutes (%) | 29.6 ± 9.8 | 51.7 ± 22.1 | P = 0.138 |

### Example 8—Vascular Functional Testing

Short-Term Blood Flow Studies

In vitro, each graft was connected to a circuit composed of silicone tubing, a pressure transducer, and a peristaltic pump. Recirculation of freshly harvested, heparinized porcine blood was targeted at 9-12 mmHg to mimic maximum physiologically achievable venous pressure. In vivo characterization was achieved using domestic pigs weighing 30-35 kg utilizing both the described surgical models, and silicone tubing was utilized to connect the venous conduits, allowing for direct measurement of blood flow with and without the presence of the graft in the circuit. Intravenous (IV) heparin was used to maintain an activated clotting time (ACT)>600 seconds throughout the procedure.

Baseline blood flow was measured before the graft was added to the circuit. A percentile blood flow (PBF) was calculated to reflect the ratio of blood flow through the graft compared to baseline at 10-15 minute intervals for 30-60 minutes. Flow was assessed through direct measurement of collected outflow blood for 60 seconds, which was subsequently returned to the circuit. Post-test graft venogram through the portal vein was performed to assess patency of the portal vascular tree using 15-20 ml of Omnipaque 3000.

Long-Term Assessment of Vascular Patency

Grafts with >30 mg/hr GCR were transplanted using the portal vein surgical model. Due to noted positive cytotoxicity tests between HUVECs and Naive pig sera suggestive of potential xeno-incompatibility, pigs were divided into two groups by block randomization. The first group underwent graft implantation with no added immunosuppression while the second group had the addition of a steroid-based immunosuppression protocol with splenectomy performed prior to graft implantation. Post-operative course of the pigs in both groups was otherwise identical.

The surgical procedure was performed maintaining normal hemostasis with no use of systemic heparinization. Side clamping of the pigs' portal vein and Vena cavae was performed to allow the anastomoses to be fashioned minimizing the risk of thrombosis. After the surgical procedure, the pigs were monitored in a recovery cage and assessed every 4-6 hours for any signs of bleeding or immediate surgical complications. Pigs were allowed to drink during this period as tolerated. Subsequently they were allowed to return to the regular housing and allowed regular diet as tolerated.

Operative data included length of the OR, cold ischemia time, ACT, liver function test (LFT), complete blood count (CBC), coagulation factors, and cytotoxicity profile were followed pre-operatively as well as at post-operative days 1, 3, 7, 10, 15, and 20 as long as the graft showed radiological evidence of vascular patency and parenchymal perfusion. To evaluate vascular patency and graft perfusion, contrast enhanced computed tomography (CT) scans were performed serially postoperatively following the same time points listed above.

All pigs were followed until graft thrombosis was ascertained via contrast enhanced scans, except for one pig in the second group which was intentionally euthanized at post-operative day 7 for the purpose of obtaining histopathological data of the graft at that time.

Results

Advanced CT imaging with intravenous contrast was used to characterize the ability of implanted revascularized grafts to sustain continuous perfusion following implantation. Revascularized liver grafts were implanted into 7 pigs for long-term studies without the use of heparin or anti-platelet therapies or immunosuppression (FIG. 3B). Early graft thrombosis, within 24 hours of implantation, was observed in 3 of the grafts. The remaining 4 grafts were imaged via CT on Day 0, Day 1, Day 3, Day 7 and Day 10. Evidence of graft perfusion was present in 75% of the grafts at day 3 and only 25% of the grafts at day 7 with the last graft only demonstrating limited perfusion on Day 7 with no perfusion by Day 10 (FIG. 3C). Volumetric reconstruction was performed and demonstrated the implantation location and active perfusion post implantation (FIG. 3D).

### Example 9—Cytotoxicity and Immune Characterization

Xeno-Compatability

48-well flat plates were seeded with 1×10⁵ cells/well and incubated at 37° C. until confluence of 80-100% was reached (24-48 hours). Following an initial PBS wash, pig sera were diluted 1:4 with EBM media and 200 μl of diluted pig sera were added to each well. Following 30 minutes of incubation at room temperature, wells were washed with PBS, and 200 μl of unadsorbed rabbit complement diluted 1:16 with EBM were added to each well and incubated at room temperature for 1 hour. 2 μl of 1% Fluoroquench were added for fluoroscopic assessment of viable and nonviable cells. Cytotoxicity was characterized by the percentage of nonviable cells and samples were classified as negative (0-19%), weak (20-50%), intermediate (51-80%), or strong (80-100%), respectively.

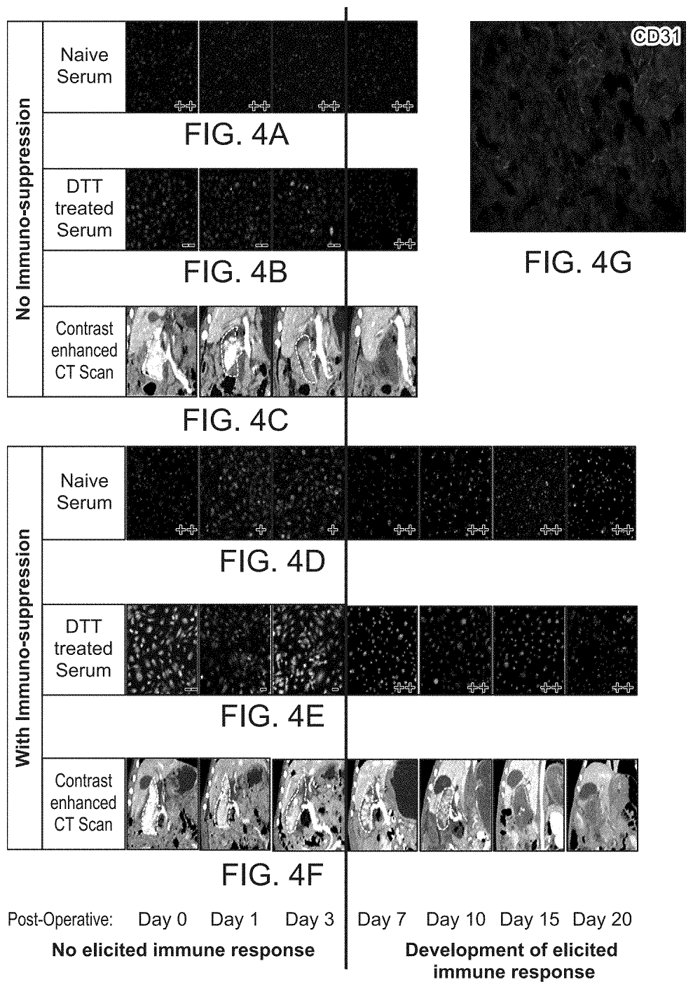

The native pig immune response to HUVEC cells was characterized to determine the role in the observed high percent of graft failure between Day 3 and Day 7. Pig serum was collected at the time of each CT scan and used in a cytotoxicity assay [25]. High level of cytotoxicity between naïve pig sera and HUVECs at baseline was noted (FIG. 4A). The addition of dithiothreitol (DTT) deactivated the IgM-related cytotoxicity resulting in no/low cytotoxicity at Day 0, 1, and 3, but a drastic increase in cytotoxicity at day 7 was noted (FIG. 4B), demonstrating a large native immune response to the implanted HUVEC cells. The IgM independent immune response correlated with a loss of graft perfusion in all of the implanted grafts following Day 3 (FIG. 4C).

### Example 10—Post-Operative Immunosuppression

Immunosuppression utilizing a splenectomy and IV steroids was employed to further determine the effect on continuous perfusion through the liver grafts. At Day 0, prior to graft implantation, surgical splenectomy and intravenous methylprednisolone was administrated at 500 mg with subsequent daily doses of 500 mg, 250 mg, 250 mg, 125 mg, 125 mg, 80 mg, 60 mg, 40 mg 30 mg and 20 mg through Day 10 respectively. Revascularized liver grafts were implanted into 5 pigs via portal anastomosis for long term studies without the use of heparin or anti-platelet therapies. Early graft thrombosis, within 24 hours of implantation, was observed in only 1 of the 5 grafts. The resulting 4 grafts were imaged via CT on days 0, 1, 3, 7, 10, 15, and 20.

Evidence of graft perfusion was present in 100% of the grafts at day 7, demonstrating a significant improvement over the non-immune suppressed group. One graft was harvested at day 7 for histology and the other 3 grafts were monitored for flow, with 2 grafts demonstrating limited flow on days 15 and 20. The group with immunosuppression had significantly longer graft perfusion and vascular patency when compared to the group without immunosuppression, 9.8±3.8 days versus 3.5±2.5 days, p=0.033, respectively (Table 3).

TABLE 3
Comparison of relevant surgical variables, resulting flow and long term patency monitored through
contrast enhanced CT scans between immunosuppressed and non-immunosuppressed groups. Immune
suppressed animals demonstrated a significant increase in graft perfusion duration.

| | | | |
|---|---|---|---|
| Variable | No immune -suppression | With immune suppression | Statistical significance |

Surgical Characteristics of Host and Graft
Host variables

| | | | |
|---|---|---|---|
| Pig Weight (Kg) | 35.75 ± 3 | 36.1 ± 3.6 | P = 0.9 |
| Arterial blood pressure (MAP) - | 63.3 ± 20 | 38.3 ± 13.6 | P = 0.13 |
| Before graft perfusion (mmHg) | | | |
| Arterial blood pressure (MAP) -30- | 65 ± 11.17 | 61.3 ± 15.9 | P = 0.73 |
| minutes after graft perfusion (mmHg) | | | |
| Activated clotting time (ACT) - Prior to | 134.25 ± 9.3 | 128.5 ± 38.7 | P = 0.79 |
| graft perfusion (sec) | | | |
| Activated clotting time (ACT) - 30- | 140.75 ± 15.9 | 142.75 ± 24.5 | P = 0.9 |
| minutes after graft perfusion (sec) | | | |
| Portal vein native blood flow (ml/min) | 482.5 ± 160.9 | 393.3 ± 100.7 | P = 0.44 |

Graft Variables

| | | | |
|---|---|---|---|
| End Glucose Consumption (EGC) (mg/hr) | 49.7 ± 11.4 | 49.31 ± 28.1 | P = 0.98 |
| Peak Glucose Consumption (PGC) (mg/hr) | 60.5 ± 21 | 65.7 ± 16 | P = 0.7 |
| Duration in culture (days) | 19.3 ± 4 | 22.3 ± 2 | P = 0.25 |

Graft flow and long term patency
Intra-operative bloodflow

| | | | |
|---|---|---|---|
| Initial graft flow (ml/min) | 110 ± 77.46 | 46.7 ± 20.8 | P = 0.26 |
| Graft flow at 15 minutes (ml/min) | 145.75 ± 93.7 | 100 ± 34.6 | P = 0.46 |
| Graft flow at 30 minutes (ml/min) | 108.75 ± 65 | 180 ± 53 | P = 0.11 |

Long-term patency

| | | | |
|---|---|---|---|
| Days with evidence of graft perfusion on | 3.5 ± 2.5 | 9.75 ± 3.8 | P = 0.033 |
| CT scan (days) | | | |

Complement-dependent cytotoxicity in both groups were similar at Day 0, however the cytotoxicity profile post-operatively provided a different response. In the immunosuppressed group, the cytotoxicity was significantly lower in the first 7 days post-operatively compared to the non-immunosuppressed group (FIGS. 4 B and D). At post-operative days 3-7 there was evidence of non-IgM-dependent complement-mediated cytotoxicity, not inhibited by the addition of DTT, reflecting the development of an elicited immune response to HUVECs (FIG. 4E). These immunological findings correlate very well with the gradual loss of graft perfusion and eventual thrombosis (FIG. 4F). Immunostaining of an explanted graft from a pig in the immunosuppression group at Day 7 demonstrated the presence of human endothelial cells and patent vasculature (FIG. 4G) were no human endothelial cells were present in a Day 7 graft from a pig in the non-immunosuppression group.

Discussion

To assess the confounding effect of a native immune response to human cells, a novel detection of the xeno-compatibility phenomenon between pig serum and human cells was employed. A non-sophisticated 10 day immunosuppression protocol was used to demonstrate the ability to further increase to vascular perfusion of the revascularized liver grafts from 3.5 day to over 9.8 days, on average. The 10-day immunosuppression protocol used in this study was not tailored to provide long-lasting immunosuppression, but rather to determine whether a species-dependent immunological response was contributing to the loss of graft perfusion. Vascular patency was significantly prolonged using the immunosuppression regimen indicating that the xeno-incompatibility played a significant rule. Following immunosuppression withdrawal, similar kinetics for graft thrombosis were observed. Vascular patency in the humanized bioengineered liver grafts would likely have lasted longer with continued therapy.

The bioengineered grafts generated in this study provide a strong foundation for further advances in the field of bioengineering whole organs and overcome a key technical barrier. In the absence of immuno-incompetent pigs, the studies also defines a path forward for the functional evaluation of liver grafts revascularized with human endothelial cells.

### Example 11—Characterization of Vascular Patency and Ammonia Clearance

In this study, we characterize the vascular patency and ammonia clearance potential of a clinically-translatable porcine liver scaffold seeded with HUVECs and primary hepatocytes.

Methods

Whole livers were decellularized by perfusion with a series of detergent containing buffers to generate the extracellular matrix scaffolds as described in Example 1. Naked or HUVEC-seeded scaffolds were infused with porcine hepatocytes through the hepatic vein and cultured in bioreactors under continuous portal vein perfusion. Daily bioreactor media samples were used to monitor nutrient consumption and albumin production. Functional ammonia clearance kinetics were measured by sampling culture media at regular intervals following the addition of ammonium chloride to the bioreactor system. Functional graft patency was assessed by perfusing heparinized porcine blood through the graft at a constant pressure. Histology was obtained using protocols as described above with respect to Example 5.

Results

When perfused at a constant pressure during bioreactor culture, co-culture grafts maintained steady flow rates in contrast to their hepatocyte-only counterparts, which exhibited a progressive reduction in flow rates over time. Co-culture grafts exhibited enhanced ammonia clearance kinetics and higher albumin production than hepatocyte-only grafts. Finally, co-culture grafts maintained stable flow rates when perfused with blood, suggesting significant reendothelialization and the presence of a functional vasculature.

Hepatocyte function was enhanced in scaffolds seeded initially with HUVECs when compared to hepatocyte-only grafts. Co-culture grafts exhibited improved perfusion dynamics during bioreactor culture, and remained patent upon perfusion with blood. Taken together, these results suggest that reendothelialized porcine liver scaffold is a promising substrate for further recellularization with hepatocytes to obtain a transplantable functional liver graft to address the chronic need for transplantable livers.

Accordingly, perfusion of endothelial cells produces enhanced ammonia clearance relative to perfusion of hepatocytes alone, providing evidence that endothelialized perfusion recellularized liver are promising candidates for xenogeneic liver transplants.

### Example 12—Biliary Duct Hepatocyte Infusion as a Method for Functionally Repopulating Whole Decellularized Porcine Liver Matrix

Methods

Whole porcine livers were perfusion decellularized using a series of detergents as described above in Example 1. Livers were washed with PBS and disinfected with peracetic acid. Primary hepatocyte cell suspensions were infused through the bile duct or hepatic vein of decellularized liver grafts using a peristaltic pump. Seeded grafts were cultured for 2-5 days in bioreactors under continuous portal vein perfusion and culture metabolites were measured daily. Recellularized grafts were terminally fixed in formalin and stained using histological protocols as described in Example 5.

Results

Histological analysis of grafts seeded through the biliary duct revealed that hepatocytes localized primarily within the intralobular space, with relatively few cells observed within the vasculature. In contrast, grafts seeded through the hepatic vein showed significant cell enrichment within the vasculature, and comparatively lower cell density within parenchymal lobules. Grafts seeded through the biliary duct exhibited lower albumin production, lower glutamine consumption, and more rapid ammonia accumulation than hepatic vein seeded grafts in extended cultures.

Conclusions

Hepatocyte infusion through the biliary duct on decellularized porcine whole liver matrix results in significant cell enrichment within parenchymal lobules and minimal attachment in vessels, though overall graft functionality as inferred by metabolite analysis was lower than grafts seeded through the hepatic vein. Taken together, these results suggest that the biliary duct may be a viable conduit for infusion of hepatocytes or other cell types.

Example 13 through Example 18 below describe an additional study showing successful recellularization a decellularized liver using comparable methods to those described in Examples 1 to 11.

### Example 13—Decellularization of Additional Porcine Livers

Whole livers (250 to 350 grams) were excised from cadaveric pigs. The Suprahepatic Vena Cava (SVC), Inferior Vena Cava (IVC), Portal Vein (PV), and Bile Duct were cannulated and flushed with 150 ml of sterile saline. The cannulated livers were perfusion decellularized with 1× Triton X-100 for 2-5 hours followed by 0.6% sodium dodecyl sulfate for 4-8 hours at a perfusion pressure maintained between 8-12 mmHg. The decellularized livers were disinfected with 1000 ppm peracetic acid (PAA; U.S. Water, BI0032-6). The decellularized grafts were washed with phosphate buffered saline and stored. All decellularization was completed in an ISO 7 cleanroom. Decellularization and recellularization utilized a custom-built perfusion system to automatically adjust flow to maintain a defined pressure utilizing peristaltic pumps.

Decellularization and recellularization utilized a custom-built perfusion system to automatically adjust flow to maintain a defined pressure utilizing Cole-Palmer peristaltic pumps.

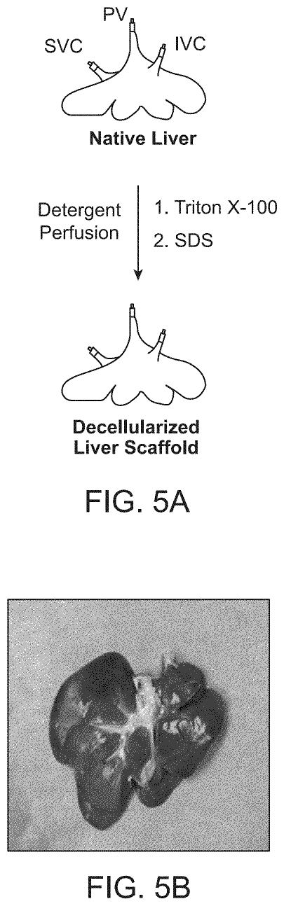

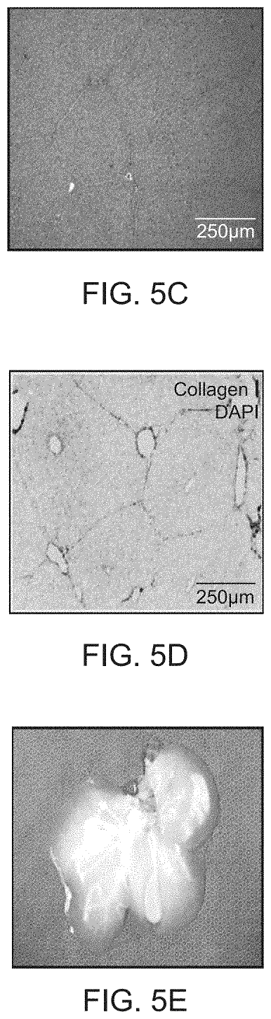

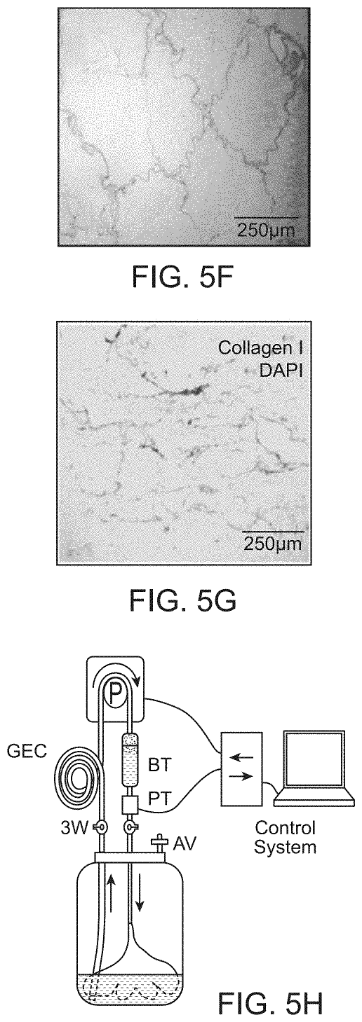

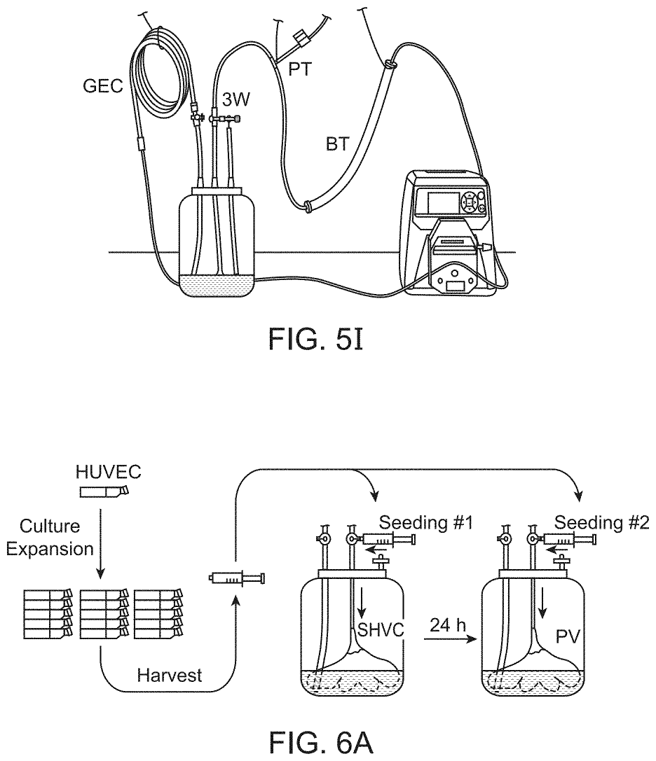

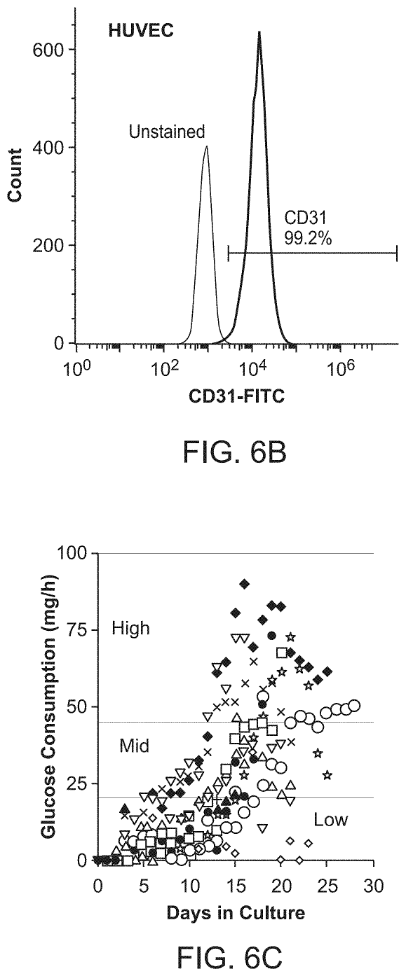

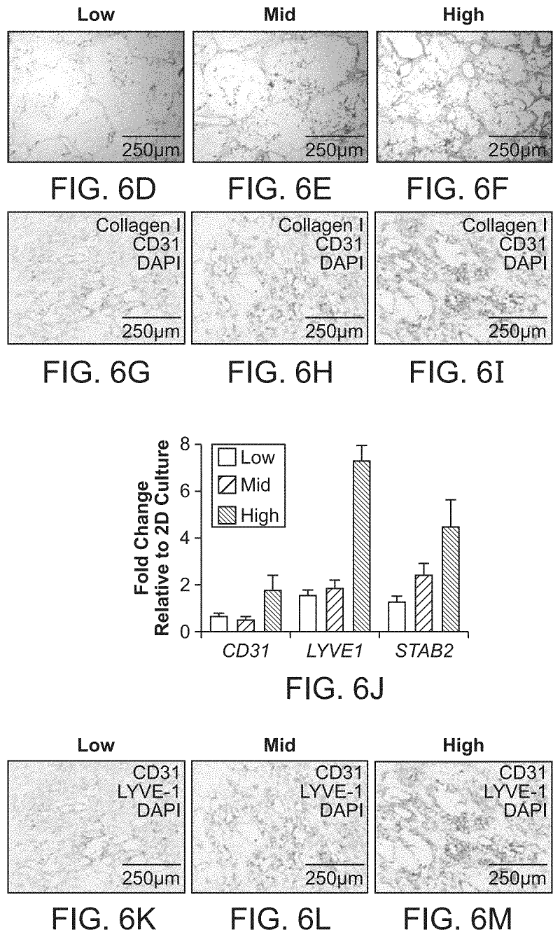

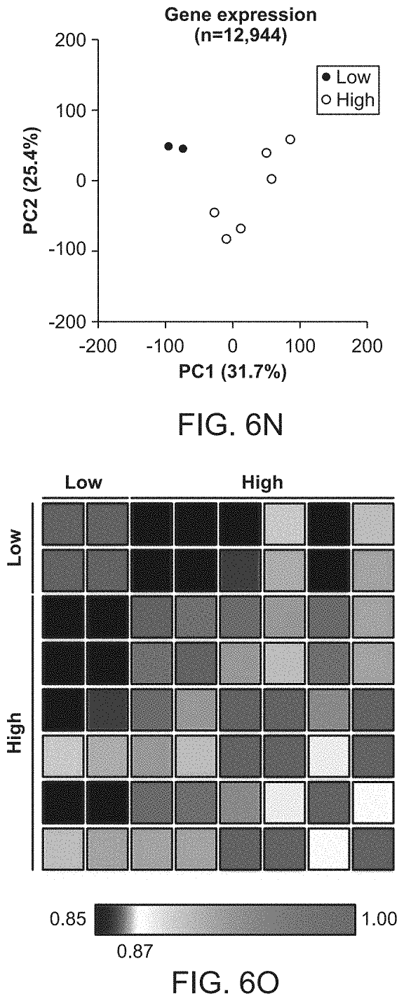

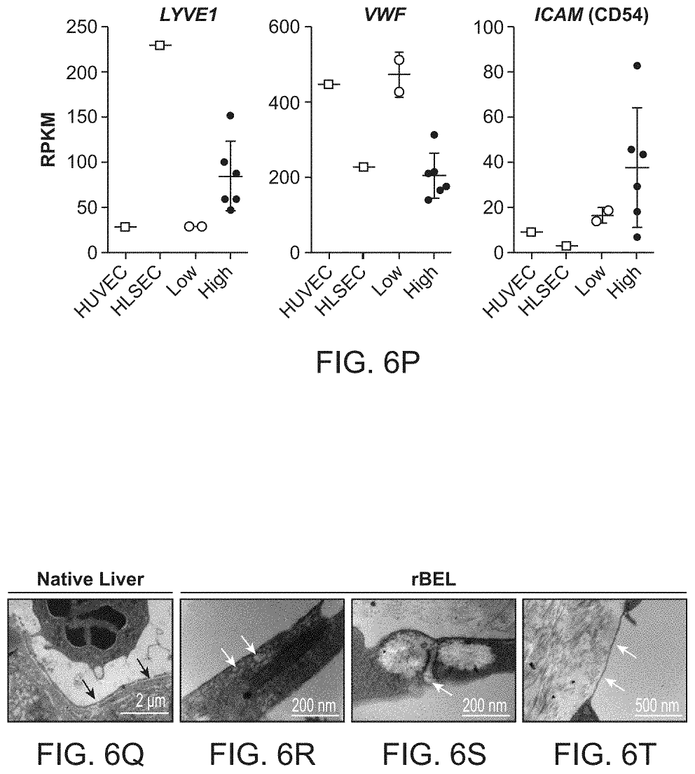

Porcine livers utilized in this study were cannulated on the portal vein (PV), infrahepatic inferior vena cava (IVC), and suprahepatic vena cava (SVC), and decellularized by sequential perfusion with Triton X-100 solutions and sodium dodecyl sulfate (SDS) solutions to remove cellular material while preserving the overall architecture of the scaffold (FIG. 5A, B, E). Histological sectioning from representative decellularized scaffolds confirmed the maintenance of parenchymal liver lobule structures when compared to that of native porcine liver tissue (FIG. 5C, F), as well as retention of Collagen I (FIG. 5D, G). Decellularized liver scaffolds were mounted in custom bioreactors (FIG. 5H, I) and perfused with culture media through the SVC at a constant inflow pressure of 12 mmHg. Following 72 h of continuous media perfusion to precondition the scaffold and confirm the absence of viable bioburden, 1.5×10⁸ HUVECs were infused through the perfusion circuit into the SVC vasculature (FIG. 6A). Following 24 h of continuous media perfusion, liver scaffolds were aseptically manipulated and infused with an additional 1.5×10⁸ HUVECs through the PV (FIG. 6A). Prior to seeding, purity of the HUVEC cultures were confirmed by CD31+ flow cytometry (FIG. 6B). Culture media was continuously perfused through the PV at 12 mmHg for the remaining period of bioreactor culture.

### Example 14—Characterization of HUVEC Proliferation and Phenotypic Plasticity in rBELs

HUVEC Cell Culture and Seeding of Decellularized Liver Constructs.

Human umbilical vein endothelial cells (Lonza, C2517A) were cultured in antibiotic-free EGM-2 (Lonza, CC-3162) medium in tissue culture flasks (Falcon) at 37° C. and 5% CO₂ and passaged with 0.25% trypsin (Thermo, 25200056) at 90-100% confluency according to manufacturer's protocol. The highest passage used for seeding liver grafts was passage 11.

The medium used for HUVEC culture was also used for seeding and maintaining revascularized liver constructs in this study. Decellularized porcine livers were placed in a custom bioreactor containing 800 ml of media, connected to the perfusion inlet via the SVC, and perfused at 12 mmHg with culture media prior to seeding. 1.5×10⁸ HUVECs HUVECs were resuspended in 100 ml of media and seeded through the SVC followed by 50 ml of fresh media to clear the measured tubing void volume. The infused cell suspension was left under static conditions for one hour and then continuous perfusion was restarted. After 24 hours, perfusion was changed from the SVC to the PV and the seeding protocol was repeated with an additional 1.5×10⁸ HUVECs. Re-endothelialized grafts were maintained in a continuous perfusion loop with metabolites (glucose, lactate, glutamine, glutamate and ammonia) monitored daily in collected media samples using a BioProfile FLEX analyzer (Nova Biomedical). Culture media was exchanged and the volume increased depending on the rate of glucose depletion in the circulating medium to ensure 24 hour glucose levels above 500 mg/L.

Histological Analysis.

Tissue samples analyzed in this study were perfused with PBS and fixed with 10% Neutral Buffered Formalin (VWR 16004-128). Fixed tissues were paraffin embedded, sectioned and stained using standard histologic techniques. Immunofluorescence slides were deparaffinized, rehydrated and retrieval was performed in citrate buffer, pH 6.0 (Abcam AB93678) in a programmable decloaker (Biocare DC2012). Slides were permeabilized with PBS+0.05% Tween-20 (Sigma P9416) and blocked with Sea Block (Thermo 37527). Primary antibodies used included mouse anti-CD31 (Abcam AB187377), rabbit anti-Collagen I (Abcam AB34710), rabbit anti-LYVE1 (Abcam AB33682), and mouse anti-C4D (Abcam AB90804). Secondary antibodies used were goat anti-rabbit Alexa Fluor 488 (Thermo A11078), goat anti-mouse Alexa Fluor 488 (Thermo A11029), and goat anti-rabbit Alexa Fluor 555 (Thermo A21429). Slides were stained with 4′,6-diamidino-2-phenylindole (Thermo D1306) diluted 1:200 in PBS and mounted using ProLong Antifade Mountant (Thermo P36961). Fluorescence slides were imaged on a Zeiss Axioskop 40 and H&E slides were imaged on an Accuscope 3012.

Results

To define quantitative markers for non-invasively monitoring endothelial cell proliferation in the liver scaffold, a panel of metabolites (glucose, lactate, glutamate, and ammonia) were measured daily from a sample of rBEL culture media. GCR measured throughout the period of bioreactor culture exhibited sigmoidal kinetics and could be generally characterized by low (<20 mg/h), mid (20-45 mg/h), and high (>45 mg/h) GCR phases (FIG. 6C). Histological examination of representative rBELs with low, mid, and high GCRs correlated with increasing endothelial cell densities as inferred by H&E staining, with evidence of primary engraftment in larger vessels and subsequent expansion and migration into the parenchymal or sinusoidal niche at mid and high GCRs (FIG. 6D-F). As a result, rBEL GCRs were utilized as a metric for estimating the extent of graft re-endothelialization in later parts of this study. During bioreactor culture, media volumes were adjusted and replaced daily to maintain steady state glucose levels above 500 mg/L (>50% of baseline media concentrations) to ensure consistent proliferation kinetics and discourage premature cell senescence due to glucose starvation.

### Example 15—Phenotypic Plasticity of HUVECs in rBELs

Transmission Electron Microscopy

Tissue was fixed with 4% paraformaldehyde+1% glutaraldehyde fix in phosphate buffered saline, pH 7.2. Following fixation, cells were stained with 1% osmium tetroxide and 2% uranyl acetate, dehydrated through an ethanol series and embedded into Embed 812 resin. After a 24 h polymerization at 60° C., 0.1 micron ultrathin sections were post-stained with lead citrate. Micrographs were acquired using a JEOL1400+ transmission electron microscope (Peabody, Mass.) operating at 80 kV with a Gatan Onus camera and Digital Micrograph software (Pleasanton, Calif.).

RNA Extraction and Quantitative Reverse-Transcription PCR (qRT-PCR).

RNA isolation was performed using TRIzol Reagent (Thermo Fisher) and transcribed to cDNA using the Superscript III First-Strand Synthesis System (Invitrogen). Gene expression analysis was performed using the Platinum SYBR green qRT-PCR supermix-UDG kit (Invitrogen) in a ViiA 7 Real-Time PCR instrument (Thermo Fisher Scientific). Ribosomal protein L19 (RPL19) was used as a housekeeping gene for normalization. The following primer sets were used in this study: RPL19 5′ATTGGTCTCATTGGGGTCTAAC3′, 5′AGTATGCTCAGGCTTCAGAAGA3′; STAB2 5′GCAAGAAGATGTGATAGGAAGTCTC3′, 5′ACAACACCGAGGTTGGAGAT3′, LYVE1 5′ TTTGCAGCCTATTGTTACAACTCAT3′, 5′GGGATGCCACCCAGTAGGTA3′ and CD31 5′ TCTGCACTG CAGGTATTGACAA, 5′CTGATCGATTCGCAACGGA3′.

RNA-Seq Analysis.

Tissue samples from low GCR (n=2) and high GCR (n=8), along with HUVECs (n=1) and human liver sinusoidal endothelial cells (LSECs) (Cell Systems, ACBRI 566) (n=1+ were processed for RNA-seq analysis. RNA isolation was performed using TRIzol Reagent (Thermo Fisher). mRNA isolation for all samples was performed using the Direct-zol RNA Miniprep Kit (Zymo Research) and quantified using a NanoDrop 2000 spectrophotometer (Thermo Fisher).

Samples were assessed for RNA integrity (RIN) using the Agilent Bioanalyzer DNA 1000 chip (Invitrogen). Only samples with RIN scores >6 and DV₂₀₀>50% were selected for sequencing. RNA-sequencing and subsequent primary and secondary data analysis was performed as previously described. In brief, library preparation was performed using the TruSeq RNA library preparation kit (Illumina). Polyadenylated mRNAs were selected using oligo dT magnetic beads. TruSeq Kits were used for indexing to permit multiplex sample loading on the flow cells and paired-end sequencing reads were generated on the Illumina HiSeq 2000 sequencer. Quality control for concentration and library size distribution was performed using an Agilent Bioanalyzer DNA 1000 chip and Qubit fluorometry (Invitrogen). Sequence alignment of reads and determination of normalized gene counts were performed using the MAP-RSeq (v.1.2.1) workflow, utilizing TopHat 2.0.6, and HTSeq. Normalized read counts were expressed as reads per kilobasepair per million mapped reads (RPKM).

All genes with an average expression >0.3 RPKM in at least one group (n=12,944) were utilized for subsequent analyses. Principal Component Analysis (PCA) was performed using ClustVis online tool. Similarity matrix and hierarchical clustering analysis was performed using Morpheus matrix visualization and analysis. A list of input genes used for the similarity matrix analysis is depicted in Table 4 below.

TABLE 4
Similarity matrix input genes.

| Gene ID | |
|---|---|
| F8 | ENG (CD105) |
| PECAM1 (CD31) | ICAM1 (CD54) |
| STAB2 | CD34 |
| LYVE1 | CD36 |
| CD14 | TEK (TIE2) |
| VWF | |

Functional annotation and Gene Ontology (GO) term enrichment scores were calculated using DAVID Bioinformatics Resources 6.8 database (DAVID 6.8). Table 5 below depicts the results of the RNA-seq DAVID Analysis.

TABLE 5
RNA-seq DAVID Analysis

| | Enrichment Score: | | | | | | |
|---|---|---|---|---|---|---|---|
| Annotation Cluster 1 | 1.4048422219123071 | | | | | List | Pop |
| Category | Term | Count | % | PValue | Genes | Total | Hits |
| GOTERM_MF_DIRECT | GO:0015485~cholesterol | 3 | 4.615385 | 0.007333 | OSBP2, | 54 | 41 |
| | binding | | | | APOD, CETP | | |
| GOTERM_BP_DIRECT | GO:0006869~lipid | 3 | 4.615385 | 0.021526 | OSBP2, | 51 | 76 |
| | transport | | | | APOD, CETP | | |
| UP_KEYWORDS | Transport | 8 | 12.30769 | 0.386547 | KCNMB4, | 63 | 1978 |
| | | | | | TNFAIP8L3, | | |
| | | | | | OSBP2, | | |
| | | | | | APOD, | | |
| | | | | | KIF17, | | |
| | | | | | CETP, | | |
| | | | | | RAB13, | | |
| | | | | | GABRP | | |

| | Enrichment Score: | | | | | |
|---|---|---|---|---|---|---|
| Annotation Cluster 1 | 1.4048422219123071 | Pop | Fold | | | |
| Category | Term | Total | Enrichment | Bonferroni | Benjamini | FDR |
| GOTERM_MF_DIRECT | GO:0015485~cholesterol | 16881 | 22.87398374 | 0.561488544 | 0.561489 | 8.005038 |
| | binding | | | | | |
| GOTERM_BP_DIRECT | GO:0006869~lipid | 16792 | 12.99690402 | 0.999732315 | 0.983639 | 25.99412 |
| | transport | | | | | |
| UP_KEYWORDS | Transport | 20581 | 1.321264063 | 1 | 0.998406 | 99.69757 |

| | Enrichment Score: | | | | | | |
|---|---|---|---|---|---|---|---|
| Annotation Cluster 2 | 1.250896546661819 | | | | | List | Pop |
| Category | Term | Count | % | PValue | Genes | Total | Hits |
| GOTERM_CC_DIRECT | GO:0005615~extracellular | 11 | 16.92308 | 0.007351 | S100A4, | 57 | 1347 |
| | space | | | | CTHRC1, | | |
| | | | | | CCL14, | | |
| | | | | | TNFSF11, | | |
| | | | | | APOD, | | |
| | | | | | HIST1H2BK, | | |
| | | | | | TTBK2, | | |
| | | | | | IL18, CPA3, | | |
| | | | | | CETP, | | |
| | | | | | CXCL11 | | |
| KEGG_PATHWAY | hsa04060:Cytokine- | 5 | 7.692308 | 0.010708 | CCL14, | 26 | 243 |
| | cytokine receptor | | | | TNFSF11, | | |
| | interaction | | | | IL18, | | |
| | | | | | TNFRSF19, | | |
| | | | | | CXCL11 | | |
| UP_KEYWORDS | Cytokine | 4 | 6.153846 | 0.019667 | CCL14, | 63 | 190 |
| | | | | | TNFSF11, | | |
| | | | | | IL18, | | |
| | | | | | CXCL11 | | |
| GOTERM_BP_DIRECT | GO:0006954~inflammatoty | 4 | 6.153846 | 0.103047 | CCL14, | 51 | 379 |
| | response | | | | LXN, IL18, | | |
| | | | | | CXCL11 | | |
| GOTERM_BP_DIRECT | GO:0006955~immune | 4 | 6.153846 | 0.12992 | CCL14, | 51 | 421 |
| | response | | | | TNFSF11, | | |
| | | | | | IL18, | | |
| | | | | | CXCL11 | | |
| GOTERM_CC_DIRECT | GO:0005576~extracellular | 8 | 12.30769 | 0.222589 | CCL14, | 57 | 1610 |
| | region | | | | TNFSF11, | | |
| | | | | | APOD, IL18, | | |
| | | | | | MTRNR2L10, | | |
| | | | | | CPA3, | | |
| | | | | | CETP, | | |
| | | | | | CXCL11 | | |
| UP_KEYWORDS | Secreted | 8 | 12.30769 | 0.379929 | CTHRC1, | 63 | 1965 |
| | | | | | CCL14, | | |
| | | | | | TNFSF11, | | |
| | | | | | APOD, IL18, | | |
| | | | | | MTRNR2L10, | | |
| | | | | | CETP, | | |
| | | | | | CXCL11 | | |

| | Enrichment Score: | | | | | |
|---|---|---|---|---|---|---|
| Annotation Cluster 2 | 1.250896546661819 | Pop | Fold | | | |
| Category | Term | Total | Enrichment | Bonferroni | Benjamini | FDR |
| GOTERM_CC_DIRECT | GO:0005615~extracellular | 18224 | 2.610922257 | 0.521854987 | 0.521855 | 7.863182 |
| | space | | | | | |
| KEGG_PATHWAY | hsa04060:Cytokine- | 6879 | 5.443969611 | 0.458630508 | 0.458631 | 10.12167 |
| | cytokine receptor | | | | | |
| | interaction | | | | | |
| UP_KEYWORDS | Cytokine | 20581 | 6.877527151 | 0.943871332 | 0.943871 | 21.0064 |
| GOTERM_BP_DIRECT | GO:0006954~inflammatoty | 16792 | 3.474985773 | 1 | 0.983606 | 77.78453 |
| | response | | | | | |
| GOTERM_BP_DIRECT | GO:0006955~immune | 16792 | 3.128312608 | 1 | 0.991624 | 85.41479 |
| | response | | | | | |
| GOTERM_CC_DIRECT | GO:0005576~extracellular | 18224 | 1.58866732 | 1 | 0.993498 | 93.88642 |
| | region | | | | | |
| UP_KEYWORDS | Secreted | 20581 | 1.330005251 | 1 | 0.999022 | 99.65648 |

| | Enrichment Score: | | | | | | |
|---|---|---|---|---|---|---|---|
| Annotation Cluster 3 | 1.0044733114706652 | | | | | List | Pop |
| Category | Term | Count | % | PValue | Genes | Total | Hits |
| GOTERM_BP_DIRECT | GO:0006614~SRP- | 3 | 4.615385 | 0.031918 | RPS27, | 51 | 94 |
| | dependent cotranslational | | | | RPS12, | | |
| | protein targeting to | | | | RPL28 | | |
| | membrane | | | | | | |
| GOTERM_BP_DIRECT | GO:0019083~viral | 3 | 4.615385 | 0.043889 | RPS27, | 51 | 112 |
| | transcription | | | | RPS12, | | |
| | | | | | RPL28 | | |
| GOTERM_BP_DIRECT | GO:0000184~nuclear- | 3 | 4.615385 | 0.04893 | RPS27, | 51 | 119 |
| | transcribed mRNA | | | | RPS12, | | |
| | catabolic process, | | | | RPL28 | | |
| | nonsense-mediated decay | | | | | | |
| GOTERM_BP_DIRECT | GO:0006413~translational | 3 | 4.615385 | 0.062787 | RPS27, | 51 | 137 |
| | initiation | | | | RPS12, | | |
| | | | | | RPL28 | | |
| KEGG_PATHWAY | hsa03010:Ribosome | 3 | 4.615385 | 0.086631 | RPS27, | 26 | 136 |
| | | | | | RPS12, | | |
| | | | | | RPL28 | | |
| UP_KEYWORDS | Ribosomal protein | 3 | 4.615385 | 0.107193 | RPS27, | 63 | 185 |
| | | | | | RPS12, | | |
| | | | | | RPL28 | | |
| GOTERM_BP_DIRECT | GO:0006364~rRNA | 3 | 4.615385 | 0.133316 | RPS27, | 51 | 214 |
| | processing | | | | RPS12, | | |
| | | | | | RPL28 | | |
| GOTERM_MF_DIRECT | GO:0003735~structural | 3 | 4.615385 | 0.153882 | RPS27, | 54 | 222 |
| | constituent of ribosome | | | | RPS12, | | |
| | | | | | RPL28 | | |
| GOTERM_BP_DIRECT | GO:0006412~translation | 3 | 4.615385 | 0.173725 | RPS27, | 51 | 253 |
| | | | | | RPS12, | | |
| | | | | | RPL28 | | |
| UP_KEYWORDS | Ribonucleoprotein | 3 | 4.615385 | 0.224117 | RPS27, | 63 | 296 |
| | | | | | RPS12, | | |
| | | | | | RPL28 | | |
| GOTERM_MF_DIRECT | GO:0044822~poly(A) | 6 | 9.230769 | 0.279717 | S100A4, | 54 | 1129 |
| | RNA binding | | | | RPS27, | | |
| | | | | | RPS12, | | |
| | | | | | HERC5, | | |
| | | | | | SYF2, RPL28 | | |

| | Enrichment Score: | | | | | |
|---|---|---|---|---|---|---|
| Annotation Cluster 3 | 1.0044733114706652 | Pop | Fold | | | |
| Category | Term | Total | Enrichment | Bonferroni | Benjamini | FDR |
| GOTERM_BP_DIRECT | GO:0006614~SRP- | 16792 | 10.50813517 | 0.99999527 | 0.953366 | 36.15583 |
| | dependent cotranslational | | | | | |
| | protein targeting to | | | | | |
| | membrane | | | | | |
| GOTERM_BP_DIRECT | GO:0019083~viral | 16792 | 8.819327731 | 0.999999957 | 0.966392 | 46.25098 |
| | transcription | | | | | |
| GOTERM_BP_DIRECT | GO:0000184~nuclear- | 16792 | 8.300543747 | 0.999999994 | 0.957597 | 50.04127 |
| | transcribed mRNA | | | | | |
| | catabolic process, | | | | | |
| | nonsense-mediated decay | | | | | |
| GOTERM_BP_DIRECT | GO:0006413~translational | 16792 | 7.209961357 | 1 | 0.969851 | 59.22118 |
| | initiation | | | | | |
| KEGG_PATHWAY | hsa03010:Ribosome | 6879 | 5.836255656 | 0.994287338 | 0.924418 | 59.26966 |
| UP_KEYWORDS | Ribosomal protein | 20581 | 5.297554698 | 0.999999928 | 0.962678 | 73.9737 |
| GOTERM_BP_DIRECT | GO:0006364~rRNA | 16792 | 4.615722925 | 1 | 0.988969 | 86.18291 |
| | processing | | | | | |
| GOTERM_MF_DIRECT | GO:0003735~structural | 16881 | 4.224474474 | 0.999999993 | 0.998047 | 84.95571 |
| | constituent of ribosome | | | | | |
| GOTERM_BP_DIRECT | GO:0006412~translation | 16792 | 3.904208324 | 1 | 0.996107 | 92.86218 |
| UP_KEYWORDS | Ribonucleoprotein | 20581 | 3.310971686 | 1 | 0.983232 | 95.08295 |
| GOTERM_MF_DIRECT | GO:0044822~poly(A) | 16881 | 1.661352229 | 1 | 0.997812 | 97.57514 |
| | RNA binding | | | | | |

| | Enrichment Score: | | | | | | |
|---|---|---|---|---|---|---|---|
| Annotation Cluster 4 | 0.6947561688698921 | | | | | List | Pop |
| Category | Term | Count | % | PValue | Genes | Total | Hits |
| UP_SEQ_FEATURE | domain:EF-hand 1 | 3 | 4.615385 | 0.108834 | S100A4, | 62 | 185 |
| | | | | | S100A3, | | |
| | | | | | MYL6B | | |
| UP_SEQ_FEATURE | domain:EF-hand 2 | 3 | 4.615385 | 0.108834 | S100A4, | 62 | 185 |
| | | | | | S100A3, | | |
| | | | | | MYL6B | | |
| INTERPRO | IPR011992:EF-hand-like | 3 | 4.615385 | 0.211293 | S100A4, | 58 | 279 |
| | domain | | | | S100A3, | | |
| | | | | | MYL6B | | |
| GOTERM_MF_DIRECT | GO:0005509~calcium ion | 3 | 4.615385 | 0.664601 | S100A4, | 54 | 717 |
| | binding | | | | S100A3, | | |
| | | | | | MYL6B | | |

| | Enrichment Score: | | | | | |
|---|---|---|---|---|---|---|
| Annotation Cluster 4 | 0.6947561688698921 | Pop | Fold | | | |
| Category | Term | Total | Enrichment | Bonferroni | Benjamini | FDR |
| UP_SEQ_FEATURE | domain:EF-hand 1 | 20063 | 5.247515257 | 1 | 0.999982 | 76.11891 |
| UP_SEQ_FEATURE | domain:EF-hand 2 | 20063 | 5.247515257 | 1 | 0.999982 | 76.11891 |
| INTERPRO | IPR011992:EF-hand-like | 18559 | 3.440674824 | 1 | 1 | 94.18186 |
| | domain | | | | | |
| GOTERM_MF_DIRECT | GO:0005509~calcium ion | 16881 | 1.307996281 | 1 | 0.999999 | 99.99958 |
| | binding | | | | | |

| | Enrichment Score: | | | | | | |
|---|---|---|---|---|---|---|---|
| Annotation Cluster 5 | 0.6157578579482629 | | | | | List | Pop |
| Category | Term | Count | % | PValue | Genes | Total | Hits |
| SMART | SM00233:PH | 3 | 4.615385 | 0.18555 | OSBP2, | 31 | 264 |
| | | | | | SNTB1, | | |
| | | | | | ARHGAP25 | | |
| INTERPRO | IPR001849:Pleckstrin | 3 | 4.615385 | 0.202311 | OSBP2, | 58 | 271 |
| | homology domain | | | | SNTB1, | | |
| | | | | | ARHGAP25 | | |
| INTERPRO | IPR011993:Pleckstrin | 3 | 4.615385 | 0.378658 | OSBP2, | 58 | 427 |
| | homology-like domain | | | | SNTB1, | | |
| | | | | | ARHGAP25 | | |

| | Enrichment Score: | | | | | |
|---|---|---|---|---|---|---|
| Annotation Cluster 5 | 0.6157578579482629 | Pop | Fold | | | |
| Category | Term | Total | Enrichment | Bonferroni | Benjamini | FDR |
| SMART | SM00233:PH | 10057 | 3.686583578 | 0.998595044 | 0.998595 | 83.09508 |
| INTERPRO | IPR001849 :Pleckstrin | 18559 | 3.54224456 | 1 | 1 | 93.33625 |
| | homology domain | | | | | |
| INTERPRO | IPRO11993 :Pleckstrin | 18559 | 2.248122426 | 1 | 1 | 99.66613 |
| | homology-like domain | | | | | |

| | Enrichment Score: | | | | | | |
|---|---|---|---|---|---|---|---|
| Annotation Cluster 6 | 0.21409958858425263 | | | | | List | Pop |
| Category | Term | Count | % | PValue | Genes | Total | Hits |
| UP_SEQ_FEATURE | signal peptide | 12 | 18.46154 | 0.440353 | CTHRC1, | 62 | 3346 |
| | | | | | FAM174B, | | |
| | | | | | CCL14, | | |
| | | | | | MPZ, APOD, | | |
| | | | | | LAYN, | | |
| | | | | | CPA3, | | |
| | | | | | TNFRSF19, | | |
| | | | | | CETP, | | |
| | | | | | CXCL11, | | |
| | | | | | GPNMB, | | |
| | | | | | GABRP | | |
| UP_KEYWORDS | Glycoprotein | 15 | 23.07692 | 0.514225 | CTHRC1, | 63 | 4551 |
| | | | | | KCNMB4, | | |
| | | | | | FAM174B, | | |
| | | | | | ADORA3, | | |
| | | | | | MPZ, | | |
| | | | | | TNFSF11, | | |
| | | | | | CCL14, | | |
| | | | | | APOD, | | |
| | | | | | HIST1H2BK, | | |
| | | | | | LAYN, | | |
| | | | | | TNFRSF19, | | |
| | | | | | CETP, | | |
| | | | | | PHEX, | | |
| | | | | | GPNMB, | | |
| | | | | | GABRP | | |
| UP_KEYWORDS | Disulfide bond | 11 | 16.92308 | 0.599648 | S100A3, | 63 | 3434 |
| | | | | | CCL14, | | |
| | | | | | ADORA3, | | |
| | | | | | MPZ, APOD, | | |
| | | | | | LAYN, | | |
| | | | | | CPA3, | | |
| | | | | | TNFRSF19, | | |
| | | | | | CETP, | | |
| | | | | | CXCL11, | | |
| | | | | | GABRP | | |
| UP_SEQ_FEATURE | disulfide bond | 9 | 13.84615 | 0.678729 | CCL14, | 62 | 2917 |
| | | | | | ADORA3, | | |
| | | | | | MPZ, APOD, | | |
| | | | | | LAYN, | | |
| | | | | | CPA3, | | |
| | | | | | TNFRSF19, | | |
| | | | | | CXCL11, | | |
| | | | | | GABRP | | |
| UP_KEYWORDS | Signal | 12 | 18.46154 | 0.733788 | CTHRC1, | 63 | 4160 |
| | | | | | FAM174B, | | |
| | | | | | CCL14, | | |
| | | | | | MPZ, APOD, | | |
| | | | | | LAYN, | | |
| | | | | | CPA3, | | |
| | | | | | TNFRSF19, | | |
| | | | | | CETP, | | |
| | | | | | CXCL11, | | |
| | | | | | GPNMB, | | |
| | | | | | GABRP | | |
| UP_SEQ_FEATURE | glycosylation site:N- | 12 | 18.46154 | 0.767866 | KCNMB4, | 62 | 4234 |
| | linked (GlcNAc . . .) | | | | CTHRC1, | | |
| | | | | | FAM174B, | | |
| | | | | | ADORA3, | | |
| | | | | | TNFSF11, | | |
| | | | | | APOD, | | |
| | | | | | LAYN, | | |
| | | | | | TNFRSF19, | | |
| | | | | | CETP, | | |
| | | | | | GPNMB, | | |
| | | | | | PHEX, | | |
| | | | | | GABRP | | |

| | Enrichment Score: | | | | | |
|---|---|---|---|---|---|---|
| Annotation Cluster 6 | 0.21409958858425263 | Pop | Fold | | | |
| Category | Term | Total | Enrichment | Bonferroni | Benjamini | FDR |
| UP_SEQ_FEATURE | signal peptide | 20063 | 1.160538341 | 1 | 1 | 99.92639 |
| UP_KEYWORDS | Glycoprotein | 20581 | 1.07673876 | 1 | 0.999069 | 99.98106 |
| UP_KEYWORDS | Disulfide bond | 20581 | 1.046449603 | 1 | 0.999373 | 99.99809 |
| UP_SEQ_FEATURE | disulfide bond | 20063 | 0.998413085 | 1 | 1 | 99.99993 |
| UP_KEYWORDS | Signal | 20581 | 0.94235348 | 1 | 0.999377 | 99.99998 |
| UP_SEQ_FEATURE | glycosylation site:N- | 20063 | 0.917137763 | 1 | 1 | 100 |
| | linked (GlcNAc . . .) | | | | | |

| | Enrichment Score: | | | | | | |
|---|---|---|---|---|---|---|---|
| Annotation Cluster 7 | 0.18789537336586862 | | | | | List | Pop |
| Category | Term | Count | % | PValue | Genes | Total | Hits |
| GOTERM_MF_DIRECT | GO:0008270~zinc ion | 6 | 9.230769 | 0.304503 | S100A3, | 54 | 1169 |
| | binding | | | | RPS27, | | |
| | | | | | PTGR1, | | |
| | | | | | CPA3, | | |
| | | | | | PHEX, | | |
| | | | | | RNF182 | | |
| UP_KEYWORDS | Zinc | 7 | 10.76923 | 0.72467 | S100A3, | 63 | 2348 |
| | | | | | RPS27, | | |
| | | | | | CPA3, | | |
| | | | | | PRDM1, | | |
| | | | | | SNAI2, | | |
| | | | | | PHEX, | | |
| | | | | | RNF182 | | |
| UP_KEYWORDS | Metal-binding | 9 | 13.84615 | 0.879288 | S100A4, | 63 | 3640 |
| | | | | | S100A3, | | |
| | | | | | RPS27, | | |
| | | | | | CPA3, | | |
| | | | | | PRDM1, | | |
| | | | | | SNAI2, | | |
| | | | | | PHEX, | | |
| | | | | | RNF182, | | |
| | | | | | FTL | | |
| UP_KEYWORDS | Zinc-finger | 4 | 6.153846 | 0.913178 | RPS27, | 63 | 1781 |
| | | | | | PRDM1, | | |
| | | | | | SNAI2, | | |
| | | | | | RNF182 | | |

| | Enrichment Score: | | | | | |
|---|---|---|---|---|---|---|
| Annotation Cluster 7 | 0.18789537336586862 | Pop | Fold | | | |
| Category | Term | Total | Enrichment | Bonferroni | Benjamini | FDR |
| GOTERM_MF_DIRECT | GO:0008270~zinc ion | 16881 | 1.604505275 | 1 | 0.997003 | 98.36961 |
| | binding | | | | | |
| UP_KEYWORDS | Zinc | 20581 | 0.9739258 | 1 | 0.999436 | 99.99998 |
| UP_KEYWORDS | Metal-binding | 20581 | 0.807731554 | 1 | 0.999879 | 100 |
| UP_KEYWORDS | Zinc-finger | 20581 | 0.733705872 | 1 | 0.999858 | 100 |

| | Enrichment Score: | | | | | | |
|---|---|---|---|---|---|---|---|
| Annotation Cluster 8 | 0.15410234027633837 | | | | | List | Pop |
| Category | Term | Count | % | PValue | Genes | Total | Hits |
| UP_SEQ_FEATURE | topological | 11 | 16.92308 | 0.337913 | KCNMB4, | 62 | 2787 |
| | domain:Extracellular | | | | FAM174B, | | |
| | | | | | ADORA3, | | |
| | | | | | MPZ, | | |
| | | | | | TNFSF11, | | |
| | | | | | LAYN, | | |
| | | | | | TNFRSF19, | | |
| | | | | | GPNMB, | | |
| | | | | | PHEX, | | |
| | | | | | MS4A6A, | | |
| | | | | | GABRP | | |
| UP_KEYWORDS | Glycoprotein | 15 | 23.07692 | 0.514225 | CTHRC1, | 63 | 4551 |
| | | | | | KCNMB4, | | |
| | | | | | FAM174B, | | |
| | | | | | ADORA3, | | |
| | | | | | MPZ, | | |
| | | | | | TNFSF11, | | |
| | | | | | CCL14, | | |
| | | | | | APOD, | | |
| | | | | | HIST1H2BK, | | |
| | | | | | LAYN, | | |
| | | | | | TNFRSF19, | | |
| | | | | | CETP, | | |
| | | | | | PHEX, | | |
| | | | | | GPNMB, | | |
| | | | | | GABRP | | |
| UP_SEQ_FEATURE | topological | 11 | 16.92308 | 0.621085 | KCNMB4, | 62 | 3456 |
| | domain:Cytoplasmic | | | | FAM174B, | | |
| | | | | | ADORA3, | | |
| | | | | | MPZ, | | |
| | | | | | TNFSF11, | | |
| | | | | | LAYN, | | |
| | | | | | TNFRSF19, | | |
| | | | | | GPNMB, | | |
| | | | | | PHEX, | | |
| | | | | | MS4A6A, | | |
| | | | | | GABRP | | |
| GOTERM_CC_DIRECT | GO:0005887~integral | 5 | 7.692308 | 0.641472 | KCNMB4, | 57 | 1415 |
| | component of plasma | | | | MPZ, | | |
| | membrane | | | | TNFSF11, | | |
| | | | | | GPNMB, | | |
| | | | | | PHEX | | |
| UP_KEYWORDS | Membrane | 22 | 33.84615 | 0.705286 | RARRES3, | 63 | 7494 |
| | | | | | TNFAIP8L3, | | |
| | | | | | FAM174B, | | |
| | | | | | KCNMB4, | | |
| | | | | | OSBP2, | | |
| | | | | | MPZ, | | |
| | | | | | ADORA3, | | |
| | | | | | TMEM45B, | | |
| | | | | | BFSP1, | | |
| | | | | | BAALC, | | |
| | | | | | MAP1LC3B2, | | |
| | | | | | RNF182, | | |
| | | | | | WIPI1, | | |
| | | | | | TNFSF11, | | |
| | | | | | LAYN, | | |
| | | | | | SNTB1, | | |
| | | | | | TNFRSF19, | | |
| | | | | | RABB, | | |
| | | | | | PHEX, | | |
| | | | | | GPNMB, | | |
| | | | | | MS4A6A, | | |
| | | | | | GABRP | | |
| UP_KEYWORDS | Receptor | 5 | 7.692308 | 0.742023 | ADORA3, | 63 | 1648 |
| | | | | | TNFSF11, | | |
| | | | | | TNFRSF19, | | |
| | | | | | MS4A6A, | | |
| | | | | | GABRP | | |
| GOTERM_CC_DIRECT | GO:0005886~plasma | 12 | 18.46154 | 0.750525 | KCNMB4, | 57 | 4121 |
| | membrane | | | | TNFAIP8L3, | | |
| | | | | | ADORA3, | | |
| | | | | | MPZ, | | |
| | | | | | TNFSF11, | | |
| | | | | | BFSP1, | | |
| | | | | | TNFRSF19, | | |
| | | | | | RABB, | | |
| | | | | | GPNMB, | | |
| | | | | | PHEX, | | |
| | | | | | GABRP, | | |
| | | | | | GLDC | | |
| UP_SEQ_FEATURE | glycosylation site:N- | 12 | 18.46154 | 0.767866 | KCNMB4, | 62 | 4234 |
| | linked (GlcNAc . . .) | | | | CTHRC1, | | |
| | | | | | FAM174B, | | |
| | | | | | ADORA3, | | |
| | | | | | TNFSF11, | | |
| | | | | | APOD, | | |
| | | | | | LAYN, | | |
| | | | | | TNFRSF19, | | |
| | | | | | CETP, | | |
| | | | | | GPNMB, | | |
| | | | | | PHEX, | | |
| | | | | | GABRP | | |
| UP_SEQ_FEATURE | transmembrane region | 13 | 20 | 0.875601 | KCNMB4, | 62 | 5056 |
| | | | | | FAM174B, | | |
| | | | | | MPZ, | | |
| | | | | | ADORA3, | | |
| | | | | | TMEM45B, | | |
| | | | | | RNF182, | | |
| | | | | | TNFSF11, | | |
| | | | | | LAYN, | | |
| | | | | | TNFRSF19, | | |
| | | | | | PHEX, | | |
| | | | | | GPNMB, | | |
| | | | | | MS4A6A, | | |
| | | | | | GABRP | | |
| UP_KEYWORDS | Transmembrane helix | 14 | 21.53846 | 0.901882 | RARRES3, | 63 | 5634 |
| | | | | | KCNMB4, | | |
| | | | | | FAM174B, | | |
| | | | | | ADORA3, | | |
| | | | | | TMEM45B, | | |
| | | | | | MPZ, | | |
| | | | | | RNF182, | | |
| | | | | | TNFSF11, | | |
| | | | | | LAYN, | | |
| | | | | | TNFRSF19, | | |
| | | | | | PHEX, | | |
| | | | | | GPNMB, | | |
| | | | | | MS4A6A, | | |
| | | | | | GABRP | | |
| UP_KEYWORDS | Transmembrane | 14 | 21.53846 | 0.904321 | RARRES3, | 63 | 5651 |
| | | | | | KCNMB4, | | |
| | | | | | FAM174B, | | |
| | | | | | ADORA3, | | |
| | | | | | TMEM45B, | | |
| | | | | | MPZ, | | |
| | | | | | RNF182, | | |
| | | | | | TNFSF11, | | |
| | | | | | LAYN, | | |
| | | | | | TNFRSF19, | | |
| | | | | | PHEX, | | |
| | | | | | GPNMB, | | |
| | | | | | MS4A6A, | | |
| | | | | | GABRP | | |
| GOTERM_CC_DIRECT | GO:0016021~integral | 12 | 18.46154 | 0.949016 | RARRES3, | 57 | 5163 |
| | component of membrane | | | | FAM174B, | | |
| | | | | | TMEM45B, | | |
| | | | | | ADORA3, | | |
| | | | | | TNFSF11, | | |
| | | | | | LAYN, | | |
| | | | | | TNFRSF19, | | |
| | | | | | GPNMB, | | |
| | | | | | PHEX, | | |
| | | | | | RNF182, | | |
| | | | | | MS4A6A, | | |
| | | | | | GABRP | | |

| | Enrichment Score: | | | | | |
|---|---|---|---|---|---|---|
| Annotation Cluster 8 | 0.15410234027633837 | Pop | Fold | | | |
| Category | Term | Total | Enrichment | Bonferroni | Benjamini | FDR |
| UP_SEQ_FEATURE | topological | 20063 | 1.277202912 | 1 | 1 | 99.40541 |
| | domain:Extracellular | | | | | |
| UP_KEYWORDS | Glycoprotein | 20581 | 1.07673876 | 1 | 0.999069 | 99.98106 |
| UP_SEQ_FEATURE | topological | 20063 | 1.029966585 | 1 | 1 | 99.99942 |
| | domain:Cytoplasmic | | | | | |
| GOTERM_CC_DIRECT | GO:0005887~integral | 18224 | 1.12975017 | 1 | 0.999997 | 99.99886 |
| | component of plasma | | | | | |
| | membrane | | | | | |
| UP_KEYWORDS | Membrane | 20581 | 0.959036012 | 1 | 0.999548 | 99.99995 |
| UP_KEYWORDS | Receptor | 20581 | 0.991148482 | 1 | 0.999308 | 99.99999 |
| GOTERM_CC_DIRECT | GO:0005886~plasma | 18224 | 0.930995287 | 1 | 1 | 99.99998 |
| | membrane | | | | | |
| UP_SEQ_FEATURE | glycosylation site:N- | 20063 | 0.917137763 | 1 | 1 | 100 |
| | linked (GlcNAc . . .) | | | | | |
| UP_SEQ_FEATURE | transmembrane region | 20063 | 0.832032845 | 1 | 1 | 100 |
| UP_KEYWORDS | Transmembrane helix | 20581 | 0.811777699 | 1 | 0.999858 | 100 |
| UP_KEYWORDS | Transmembrane | 20581 | 0.809335614 | 1 | 0.999838 | 100 |
| GOTERM_CC_DIRECT | GO:0016021~integral | 18224 | 0.743101216 | 1 | 1 | 100 |
| | component of membrane | | | | | |

| | Enrichment Score: | | | | | | |
|---|---|---|---|---|---|---|---|
| Annotation Cluster 9 | 0.0783557897414018 | | | | | List | Pop |
| Category | Term | Count | % | PValue | Genes | Total | Hits |
| UP_KEYWORDS | DNA-binding | 6 | 9.230769 | 0.752515 | NUPR1, | 63 | 2050 |
| | | | | | HIST1H2BK, | | |
| | | | | | MAFB, | | |
| | | | | | PRDM1, | | |
| | | | | | SNAI2, | | |
| | | | | | MLF1 | | |
| UP_KEYWORDS | Isopeptide bond | 3 | 4.615385 | 0.862272 | HIST1H2BK, | 63 | 1132 |
| | | | | | MAFB, | | |
| | | | | | PRDM1 | | |
| UP_KEYWORDS | Ubl conjugation | 4 | 6.153846 | 0.89696 | HIST1H2BK, | 63 | 1705 |
| | | | | | MAFB, | | |
| | | | | | HERC5, | | |
| | | | | | PRDM1 | | |

| | Enrichment Score: | | | | | |
|---|---|---|---|---|---|---|
| Annotation Cluster 9 | 0.0783557897414018 | Pop | Fold | | | |
| Category | Term | Total | Enrichment | Bonferroni | Benjamini | FDR |
| UP_KEYWORDS | DNA-binding | 20581 | 0.956144019 | 1 | 0.999072 | 99.99999 |
| UP_KEYWORDS | Isopeptide bond | 20581 | 0.865766448 | 1 | 0.999835 | 100 |
| UP_KEYWORDS | Ubl conjugation | 20581 | 0.76641065 | 1 | 0.999894 | 100 |

| | Enrichment Score: | | | | | | |
|---|---|---|---|---|---|---|---|
| Annotation Cluster 10 | 0.0707878426665551 | | | | | List | Pop |
| Category | Term | Count | % | PValue | Genes | Total | Hits |
| UP_KEYWORDS | Repressor | 3 | 4.615385 | 0.535978 | MAFB, | 63 | 592 |
| | | | | | PRDM1, | | |
| | | | | | SNAI2 | | |
| UP_KEYWORDS | DNA-binding | 6 | 9.230769 | 0.752515 | NUPR1, | 63 | 2050 |
| | | | | | HIST1H2BK, | | |
| | | | | | MAFB, | | |
| | | | | | PRDM1, | | |
| | | | | | SNAI2, | | |
| | | | | | MLF1 | | |
| GOTERM_CC_DIRECT | GO:0005634~nucleus | 14 | 21.53846 | 0.889486 | S100A4, | 57 | 5415 |
| | | | | | MAFB, | | |
| | | | | | HERC5, | | |
| | | | | | SNAI2, | | |
| | | | | | MLF1, | | |
| | | | | | GLDC, | | |
| | | | | | RPS27, | | |
| | | | | | CDKN2B, | | |
| | | | | | TTBK2, | | |
| | | | | | HIST1H2BK, | | |
| | | | | | NUPR1, | | |
| | | | | | SYF2, | | |
| | | | | | GVINP1, | | |
| | | | | | PRDM1 | | |
| GOTERM_BP_DIRECT | GO:0006351~transcription, | 4 | 6.153846 | 0.941047 | NUPR1, | 51 | 1955 |
| | DNA-templated | | | | PRDM1, | | |
| | | | | | SNAI2, | | |
| | | | | | MLF1 | | |
| UP_KEYWORDS | Transcription regulation | 4 | 6.153846 | 0.97714 | NUPR1, | 63 | 2332 |
| | | | | | MAFB, | | |
| | | | | | PRDM1, | | |
| | | | | | SNAI2 | | |
| UP_KEYWORDS | Transcription | 4 | 6.153846 | 0.980707 | NUPR1, | 63 | 2398 |
| | | | | | MAFB, | | |
| | | | | | PRDM1, | | |
| | | | | | SNAI2 | | |
| UP_KEYWORDS | Nucleus | 10 | 15.38462 | 0.987592 | NUPR1, | 63 | 5244 |
| | | | | | HIST1H2BK, | | |
| | | | | | TTBK2, | | |
| | | | | | MAFB, | | |
| | | | | | SYF2, | | |
| | | | | | GVINP1, | | |
| | | | | | PRDM1, | | |
| | | | | | CCNG1, | | |
| | | | | | SNAI2, | | |
| | | | | | MLF1 | | |

| | Enrichment Score: | | | | | |
|---|---|---|---|---|---|---|
| Annotation Cluster 10 | 0.0707878426665551 | Pop | Fold | | | |
| Category | Term | Total | Enrichment | Bonferroni | Benjamini | FDR |
| UP_KEYWORDS | Repressor | 20581 | 1.655485843 | 1 | 0.999049 | 99.989 |
| UP_KEYWORDS | DNA-binding | 20581 | 0.956144019 | 1 | 0.999072 | 99.99999 |
| GOTERM_CC_DIRECT | GO:0005634~nucleus | 18224 | 0.826605757 | 1 | 1 | 100 |
| GOTERM_BP_DIRECT | GO:0006351~transcription, | 16792 | 0.673667319 | 1 | 1 | 100 |
| | DNA-templated | | | | | |
| UP_KEYWORDS | Transcription regulation | 20581 | 0.560347409 | 1 | 0.999997 | 100 |
| UP_KEYWORDS | Transcription | 20581 | 0.544925004 | 1 | 0.999998 | 100 |
| UP_KEYWORDS | Nucleus | 20581 | 0.622964416 | 1 | 0.999999 | 100 |

Results

Immunostaining of rBELs during low, mid and high GCR phases with anti-CD31 and anti-Collagen I antibodies (FIG. 6F-H) revealed HUVEC localization within vascular structures and overall uniform cell distribution within the decellularized liver matrix. Endothelial cell engraftment was primarily localized within larger vessels during the low GCR phase following seeding, followed by an increase in cell proliferation within sinusoidal regions at mid and high GCR phases. Immunostaining for LYVE1, a marker expressed by LSECs, demonstrated highest expression in the parenchymal sinusoids with little expression in larger vessels (FIG. 6I-K) consistent with native liver sinusoid staining. The expression and localization of LYVE1 was weakly detected during the low GCR phase and became progressively stronger in the mid and high GCR phases. Transcript levels of LSEC-associated markers measured by qRT-PCR at high GCR phase demonstrated an upregulation of LYVE1 (7.3-fold+/−0.62, n=4) and STAB2 (4.5-fold+/−1.13, n=4) compared to HUVEC cells in 2D culture, as well as a significant increase from low to high phases (p<0.05), while CD31 was upregulated to a lesser degree (1.8-fold+/−0.64, n=4) (FIG. 6L). Global characterization of rBEL samples from low and high GCR phases through RNA-seq analysis revealed significant changes in gene expression profiles over time as demonstrated by a global principle component analysis (FIG. 6N) and targeted similarity analysis using known liver endothelial cell markers (FIG. 6O). Further analysis of the RNA-seq datasets confirmed upregulation of LYVE1, and additionally showed downregulation of VWF and upregulation of in high GCR samples (FIG. 6P), revealing additional expression trends that resemble recently reported primary human LSEC transcript profiles. Table 6 below depicts genes upregulated in high GCR phase relative to low GCR phase.

TABLE 6
List of Upregulated genes
GeneID

| | | | | | | |
|---|---|---|---|---|---|---|
| ADORA3 | ARHGAP25 | CCNG1 | BAALC | IL18 | CORO2B | CCDC102B |
| FAM212B | KYNU | GABRP | CTHRC1 | LAYN | FAM174B | FTL |
| KIF17 | APOD | HCP5 | SNAI2 | MS4A6A | SHF | RPL28 |
| LINC00467 | CPA3 | HIST1H2BK | SNTB1 | RARRES3 | TNFAIP8L3 | BFSP1 |
| MPZ | LXN | PRDM1 | CDKN2B | TMEM45B | TTBK2 | MAFB |
| RAB13 | MLF1 | RNF182 | FLJ41200 | KCNMB4 | CETP | ZNFX1-AS1 |
| RPS27 | CXCL11 | RPS12 | GLDC | MAP1LC3B2 | NUPR1 | OSBP2 |
| S100A3 | HERC5 | TPD52L1 | LOC441461 | MYL6B | CCL14 | CXorf31 |
| S100A4 | PABPC4L | GPNMB | PTGR1 | TNFRSF19 | WIPI1 | MTRNR2L10 |
| SYF2 | C5orf62 | LOC646999 | GVINP1 | TNFSF11 | CBX3P2 | PHEX |

A hallmark feature of LSECs in normal liver tissue is the presence of plasma membrane fenestrations which enable diffusion of nutrients and waste products between the capillary vessels and the adjacent parenchymal space. To determine whether endothelial cells localized within sinusoids of rBEL constructs exhibited such features, TEM was performed on samples from native porcine liver tissue (FIG. 6Q) and high GCR phase rBELs (FIG. 6R-T). Micrographs from rBELs exhibited fenestrae-like structures similar to those observed in native porcine liver sections. The quantified dimensions of these features were consistent with those of LSEC fenestrations (100-150 nm). Collectively, these results reveal a novel dimension of HUVEC phenotypic plasticity and suggest that distinct microenvironments in decellularized liver matrix may have the capacity to direct phenotypic differentiation of endothelial cells.

The measurement and analysis of metabolic markers provided critical growth parameters that were evaluated as surrogates for re-endothelialization. PGCR not only indicates the level of rBEL endothelialization in culture, but is also predictive of in vivo performance. The resulting function of the rBEL was independent of days in culture, but instead dependent upon metabolic activity. It appears that these phases correlated with increased endothelial coverage starting in the larger vessels followed by proliferation and migration into the parenchymal space during mid and high GCR phases.

During this time, LYVE1 expression and localization was increased in the parenchymal space, with little expression in larger vessels, the inverse was observed with the localization of CD31, suggesting a degree of phenotypic plasticity of HUVECs within the decellularized liver scaffold. Global gene expression via RNA-seq analysis on isolated rBEL sections characterized a shift in gene expression between low and high glucose consuming rBELs and revealed additional gene expression trends consistent with an LSEC-like phenotype. To provide physical evidence of fenestrations, a key hallmark of LSECs, TEM evaluation of rBELs exhibited fenestrae-like features similar in size to those observed in native liver sections. The combined molecular analysis and microscopic examination support the shift towards an LSEC-like phenotype.

### Example 16—Confirmation that GCR in rBELs Correlates with Patency

Acute Patentcy

For in vitro blood perfusion studies, each rBEL was connected to a circuit composed of silicone tubing, a pressure transducer, and a peristaltic pump. Recirculation of freshly harvested, 37° C. heparinized porcine blood was targeted at 9-12 mmHg to mimic maximum physiologically achievable venous pressure and resulting flow rates were monitored over time.

In vivo acute blood studies were performed using domestic pigs weighing 30-35 kg. rBEL construct were connected to portal venous blood flow using silicone tubing and leur-lock connectors to achieve functional end-to-side anastomoses between the graft's and recipient animal's portal veins and IVCs. Leur lock connectors allowed for direct measurement of blood flow with and without the rBEL in the circuit. Flow was assessed through direct measurement of collected outflow blood for 60 seconds, which was subsequently returned to the circuit. Post-test graft venogram through the PV was performed to ensure the patency of the portal vascular tree using 15-20 ml of Omnipaque 3000. Intravenous (IV) heparin was used to maintain an activated clotting time (ACT)>600 seconds throughout the procedure.

Results

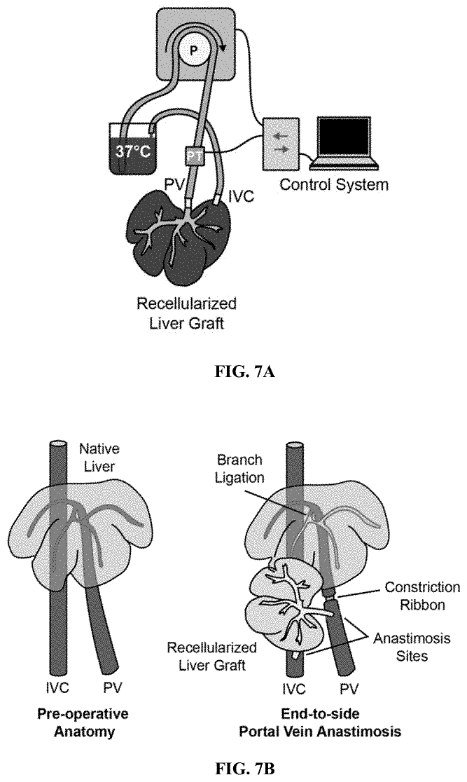

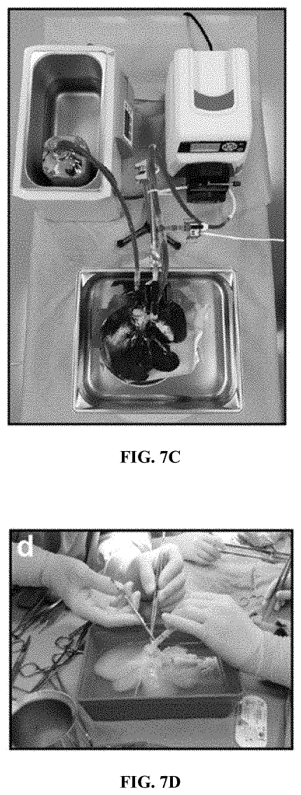

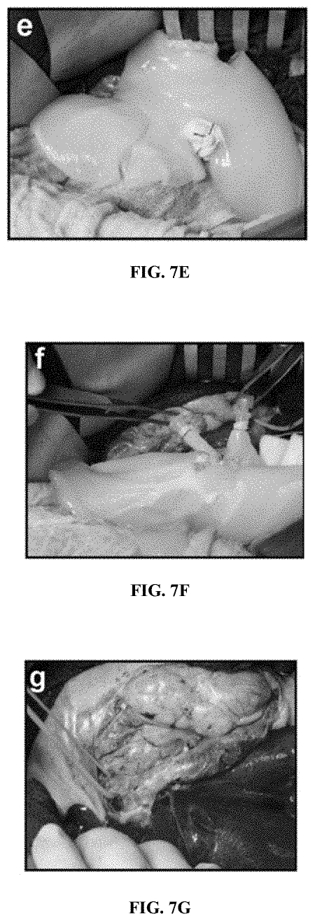

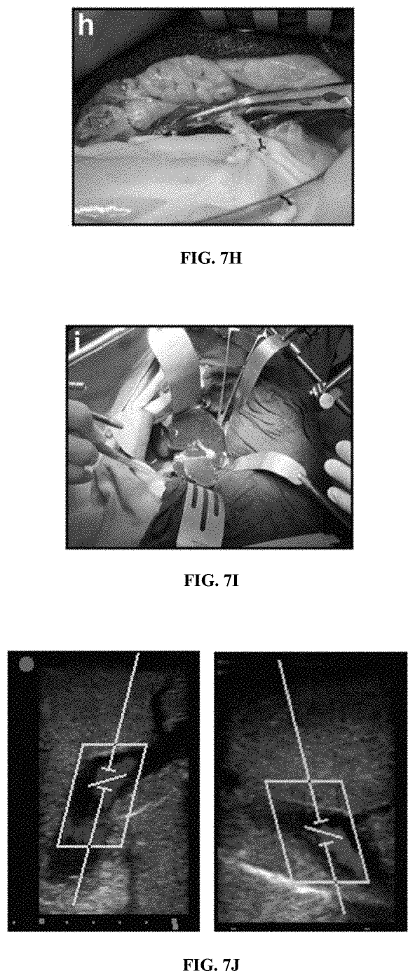

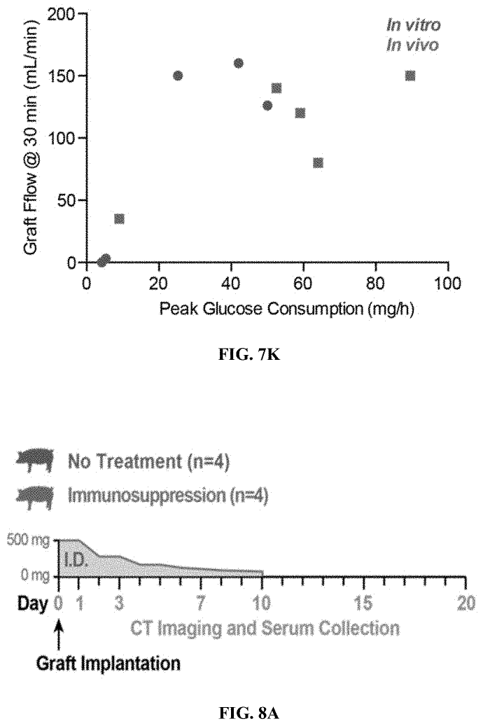

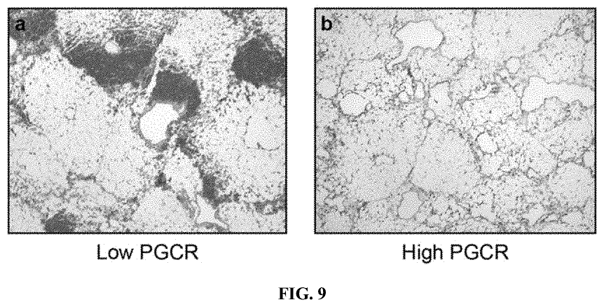

To assess the patency of rBELs, an ex vivo blood loop circuit utilizing fresh heparinized porcine blood was employed (FIG. 7A, C). Using a peristaltic pump, pre-warmed blood (37° C.) was perfused through the PV of the rBEL and returned to the blood reservoir following outflow from the IVC. Perfusion was maintained at a constant pressure of 12 mmHg and flow rates were monitored over time. Flow rates <50 ml/min after 30 minutes were deemed inadequate for in vivo perfusion. Evaluation of non-seeded decellularized liver scaffolds consistently resulted in flow rates <10 ml/min after 5 minutes and had zero flow after 15 minutes (data not shown). Peak glucose consumption rate (PGCR) rates >40 mg/h in rBELs correlated with sustained flow rates >50 ml/min, thereby validating the use of the PGCR as a marker for functional re-endothelialization of rBELs (n=5) (FIG. 7K). Histological evaluation of low glucose consuming grafts following blood perfusion and saline flushing showed blood pooling and compaction within the graft, while grafts with PGCRs>40 mg/h were efficiently cleared with saline (FIG. 9).

To determine the value of PGCR in predicting rBEL patency in vivo, a large animal porcine model for auxiliary liver transplantation was established to enable the implantation and patency assessment of rBELs (n=5). To this end, rBELs were implanted with end-to-side anastomoses between the graft's and recipient pig's portal veins (FIG. 7B, D-I). Prior to perfusing the rBELs, Portal branches supplying the pig's native liver were tied off preserving the first portal branch supplying the caudate lobe and the right lateral lobe. A constriction ribbon was also applied to the pig's portal vein distal to the anastomosis to partially bias flow through the rBEL. These measures were taken to raise the portal pressure and facilitate preferential blood flow to the implanted rBELs. The portal vein surgical model provided mean venous pressure of 8.4±2.2 mmHg (n=5) (mean+/−s.d) and mean blood flow rates of 414±16.7 ml/min (n=5) (mean+/−s.d). rBELS were implanted and monitored for 30 minutes with inflow and outflow confirmation via Doppler ultrasound (FIG. 7J). Vascular perfusion was assessed through direct measurement 30 minutes after anastomosis though direct volumetric measurement of outflow blood for 60 seconds, which was subsequently returned to the circuit. PGCR>30 mg/h demonstrated >100 ml/min of perfusion after 30 minutes in 3 of 4 rBELs (FIG. 7K) further confirming the correlation between PGCR and in vivo graft patency.

### Example 17—Long-Term In Vivo Perfusion in an Immunosuppressed Porcine Liver Transplant Model

Surgical Model

Grafts with >30 mg/h GCR were selected for transplantation in all long term in-vivo perfusion studies. Heterotopic implantation of rBELs in this study relied on end-to-side anastomoses between the graft's and recipient animal's PV, and the graft's and recipient animal's IVC. All portal branches supplying the animal's native liver with the exception of the first branch were tied off, thereby preserving blood flow to the caudate lobe and the right lateral lobe. Additionally, a constricting ribbon was applied surgically around the native portal vein distal to the anastomosis to enhance blood flow to the implanted rBEL. This sequence was followed to elevate the portal vein pressure while avoiding hemodynamic instability resulting from host portal vein clamping and abrupt cessation of portal flow.

The surgical procedure was performed under normal hemostasis without the use of systemic heparinization. Side clamping of the pigs' portal vein and vena cava were performed to allow the anastomoses to be fashioned while minimizing the risk of thrombosis. After the procedure, the pigs were monitored in a recovery cage for the first 24 hours and assessed every 4-6 hours for any signs of bleeding or immediate surgical complications. Pigs were allowed to drink during this period as tolerated. Subsequently they were allowed to return to the regular housing and allowed regular diet as tolerated.

Operative data included OR time, cold ischemia time, ACT, liver function test (LFT), complete blood count (CBC), and coagulation factors. Cytotoxicity profile were followed pre-operatively as well as at post-operative days 1, 3, 7, 10, 15, and 20. To evaluate vascular patency and graft perfusion, contrast enhanced computed tomography (CT) scans were performed serially postoperatively following the same time points listed above. All scans included a dedicated porto-venous phase taken 50-60 seconds after contrast infusion.

All pigs were followed until graft thrombosis was ascertained via contrast enhanced CT scans, except for one pig in the immunosuppressed group which was intentionally euthanized at Post-Operative day 7 for the purpose of obtaining histopathological data of the graft at that time.

Volumetric analysis of the graft perfusion and vascular patency of the large vessels was calculated using Siemens MultiModality Workstation software. Perfused areas were automatically detected through Hounsfield density cutoff threshold. Subsequent loss of perfusion was calculated over time, at day 1, 3, 7, 15 and 20. Loss of graft perfusion was defines by absence of notable perfusion outside the major portal branches and hepatic veins. Complete loss of perfusion and vascular patency was defined by a clear loss of flow in the graft's portal vein braches. Given the plasticity of the rBEL, the volume was affected in cases of ileus or gastric distension which sometimes occurred in the first 3-5 days after surgery.

Serum Cytotoxicity Assay.

The complement-based cytotoxicity assay was adapted from a previously described protocol. Briefly, 48-well tissue culture plates were seeded with 1×10⁵ HUVECs/well and incubated at 37° C. until 80-100% confluence was reached (24-48 hours). Following an initial PBS wash, pig sera were diluted 1:32 with EGM-2 media and 200 μl of diluted pig sera were added to each well. Following 30 minutes of incubation at room temperature, wells were washed with PBS, and 200 μl of unabsorbed rabbit complement (Pel-Freez) diluted 1:16 with EGM-2 media was added to each well and incubated at room temperature for 1 hour. 2 μl of 1% Fluoroquench (Thermo Fisher) was added for fluoroscopic assessment of viable and nonviable cells. Cytotoxicity was characterized by the resulting percentage of nonviable cells.

Statistical Analysis.

IBM SPSS Software version 25 was used to conduct the statistical analysis. Descriptive data are presented as mean+/−Standard deviation. For subsets of data that did not meet normality tests, the median and [range] were used. Correlations between graft flow and metabolic parameters were statistically compared using binary correlation and linear logistic regression. Surgical model parameters, duration of graft perfusion, and vascular patency between immunosuppressed and non-immunosuppressed animals were compared using a Student's t-test. Mann-Whitney test was alternatively used for subsets of data that are not normally distributed with a Shaprio-Wilk's tests of (P<0.05).

Results

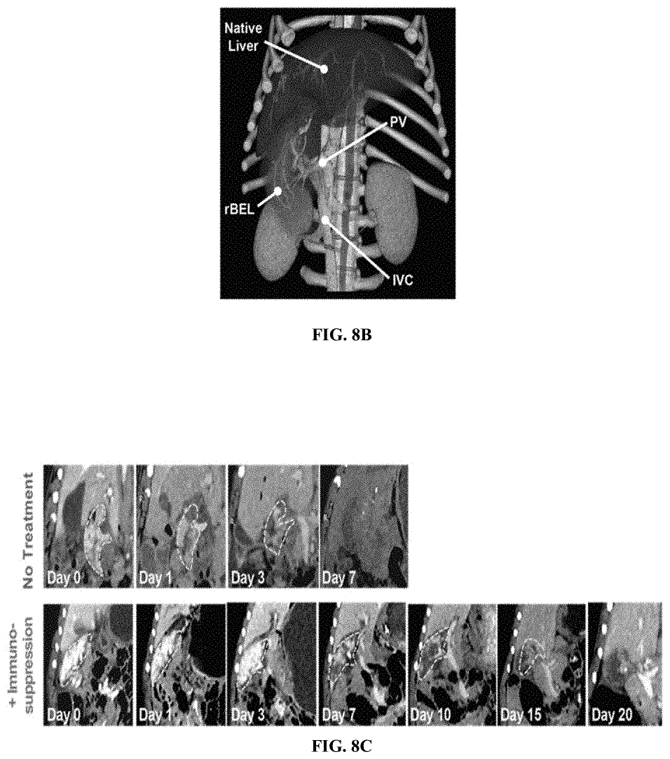

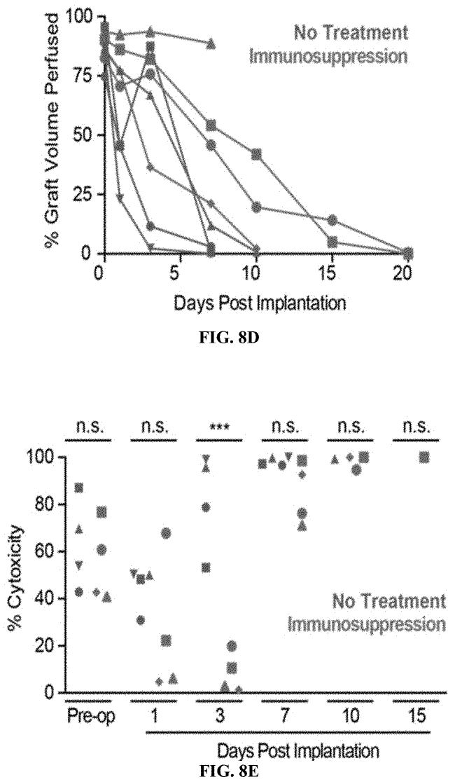

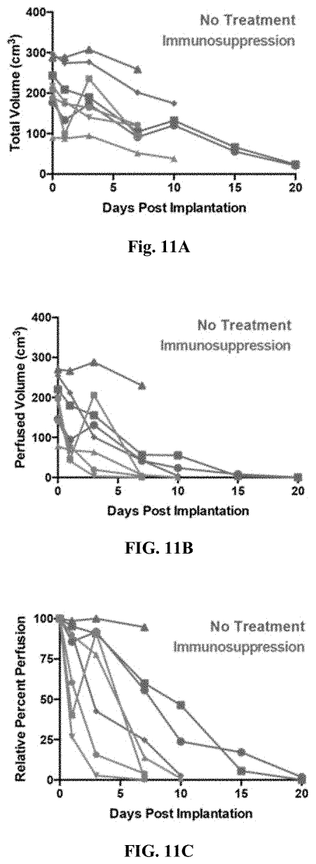

To assess long-term patency in vivo, rBELs were implanted utilizing the previously described auxiliary liver transplantation model (FIG. 7B) and recipient animals were recovered without the addition of post-operative anti-platelet or anti-coagulation therapies. To determine the impact of a host immune response directed toward the HUVEC-component of the rBELs on eventual graft failure, recipient animals were divided into two cohorts (n=4 per condition) One group underwent an immunosuppressive therapy regimen, and the other received no additional treatment (Excluded were 3 additional non-immunosuppressed implants and one additional immunosuppressed implants that experienced immediate—within 24 hours—graft loss attributable to surgical complications). In the immunosuppressed group prior to rBEL implantation, surgical splenectomy was performed and intravenous methylprednisolone was administrated at 500 mg with subsequent daily doses of 500 mg, 250 mg, 250 mg, 125 mg, 125 mg, 80 mg, 60 mg, 40 mg, 30 mg and 20 mg (FIG. 8A). CT imaging with intravenous contrast was performed post-operatively on days 0, 1, 3, 7, 10, 15, and 20 to assess the extent of perfusion through the rBELs (FIG. 8A-C). Perfusion following each CT imaging time point was quantified through computed tomography volumetric measurements using SIEMENS MultiModality Workstation Software. Graft volume was manually marked and the perfused area was auto detected through Hounsfield threshold cutoff. 3D reconstruction was performed using TeraRecon medical imaging software as well as the assistance of 3D visualizations created with Analyze (FIG. 8B). The percentage of the reduction in perfusion of the rBELs from baseline postoperative CT scan was calculated and plotted (FIG. 8D, FIG. 11). In the absence of immunosuppression, all four implanted rBELs lost >85% of their initial perfusion by day 7 post-transplant. In contrast, the immunosuppressed group had significantly longer graft perfusion and vascular patency when compared to the group without immunosuppression, 8.5 [7-15] (Median [Range]) versus 3 [1-7] days; p=0.037, and 11 [7-20] versus 3 [1-7] days; p=0.037, respectively (Table 7).

TABLE 7
Comparison of relevant surgical variables, resulting graft flow, and long-term patency monitored
through contrast enhanced CT scans between immunosuppressed and non-immunosuppressed groups.

| | | | |
|---|---|---|---|
| Variable | No Immuno-suppression | With Immuno-suppression | p - value |

Surgical Characteristics of Host and Graft
Host variables

| | | | |
|---|---|---|---|
| Pig Weight (kg) | 35.75 ± 3 | 36.1 ± 3.6 | 0.9 |
| Arterial blood pressure (MAP) - Before graft | 63.3 ± 20 | 38.3 ± 13.6 | 0.13 |
| perfusion (mmHg) | | | |
| Arterial blood pressure (MAP) - 30-minutes | 65 ± 11.17 | 61.3 ± 15.9 | 0.73 |
| after graft perfusion (mmHg) | | | |
| Activated clotting time (ACT) - Prior to graft | 134.25 ± 9.3 | 128.5 ± 38.7 | 0.79 |
| perfusion (s) | | | |
| Activated clotting time (ACT) - 30-minutes | 140.75 ± 15.9 | 142.75 ± 24.5 | 0.9 |
| after graft perfusion (s) | | | |
| Portal vein native blood flow (ml/min) | 482.5 ± 160.9 | 393.3 ± 100.7 | 0.44 |

Graft Variables

| | | | |
|---|---|---|---|
| End Glucose Consumption Rate (EGCR) | 49.7 ± 11.4 | 49.31 ± 28.1 | 0.98 |
| (mg/hr) | | | |
| Peak Glucose Consumption Rate (PGCR) | 60.5 ± 21 | 65.7 ± 16 | 0.7 |
| (mg/hr) | | | |
| Duration in culture (days) | 19.3 ± 4 | 22.3 ± 2 | 0.25 |

Graft Flow and Long Term Patency
Intra-operative blood flow

| | | | |
|---|---|---|---|
| Initial graft flow (mL/min) | 110 ± 77.46 | 46.7 ± 20.8 | 0.26 |
| Graft flow at 15 minutes (mL/min) | 145.75 ± 93.7 | 100 ± 34.6 | 0.46 |
| Graft flow at 30 minutes (mL/min) | 108.75 ± 65 | 180 ± 53 | 0.11 |

Long-term perfusion and patency

| | | | |
|---|---|---|---|
| Days with evidence of graft parenchymal | 3 [1-7] | 8.5 [7-15] | 0.037 |
| perfusion on CT scan (days)* | | | |
| Days with evidence of graft portal vein and | 3 [1-7] | 11 [7-20] | 0.037 |
| hepatic vein patency on CT scan (days)* | | | |

Blood panels were collected on each immunosuppressed and non-immunosuppressed animal prior to histological analysis (Table 8).

TABLE 8
Serial blood investigations from animals in immunosuppressed and non-immunosuppressed study groups.

Post-

| operative | No Immunosuppression | | | | | With Immunosuppression |
|---|---|---|---|---|---|---|
| day | 0 | 1 | 3 | 7 | 10 | 0 |

Complete Blood Count

| | | | | | | |
|---|---|---|---|---|---|---|
| Hemoglobin | 11.5 ± 2.6 | 7.8 ± 1.1 | 8.2 ± 0.5 | 10.2 ± 2.0 | 9.9 ± 0.9 | 11.5 ± 4.3 |
| (gm/dl) | | | | | | |
| White | 18.1 ± 3.7 | 19.4 ± 2.5 | 16.2 ± 1.8 | 25.2 ± 22.3 | 12.0 ± 1.0 | 12.7 ± 2.1 |
| Cells | | | | | | |
| (103/μ1) | | | | | | |
| Platelets | 197.3 ± 54.3 | 113.1 ± 7.9 | 146.0 ± 34.2 | 193.5 ± 143.7 | 273.5 ± 7.8 | 270.3 ± 145.1 |
| (103/μ1) | | | | | | |

Liver Function

| | | | | | | |
|---|---|---|---|---|---|---|
| Total | 0.2 ± 0.0 | 0.3 ± 0.1 | 0.3 ± 0.1 | 0.3 ± 0.0 | 0.3 ± 0.1 | 0.2 ± 0.0 |
| Bilirubin | | | | | | |
| (mg/dl) | | | | | | |
| Aspartate | 20.5 ± 3.8 | 573.3 ± 680.8 | 72.0 ± 52.7 | 55.0 ± 39.1 | 42.5 ± 17.7 | 24.5 ± 4.7 |
| Aminotransferase | | | | | | |
| (IU/L) | | | | | | |
| Alanine | 37.5 ± 15.4 | 81.3 ± 22.0 | 58.0 ± 26.1 | 64.0 ± 28.8 | 71.0 ± 1.4 | 57.0 ± 24.0 |
| Aminotransferase | | | | | | |
| (IU/L) | | | | | | |
| Alkaline | 112.5 ± 41.5 | 226.0 ± 77.5 | 94.7 ± 41.2 | 114.5 ± 66.9 | 44.2 ± 60.5 | 129.3 ± 19.8 |
| Phosphatase | | | | | | |
| (IU/L) | | | | | | |
| Gamma-Glutamyl | 29.0 ± 3.7 | 31.0 ± 3.5 | 29.0 ± 4.2 | 31.8 ± 3.9 | 32.0 ± 7.1 | 32.8 ± 5.4 |
| Transferase | | | | | | |
| (IU/L) | | | | | | |

Renal Function

| | | | | | | |
|---|---|---|---|---|---|---|
| Blood Urea | 5.5 ± 1.3 | 14.3 ± 1.5 | 6.3 ± 4.6 | 7.8 ± 6.5 | 1.9 ± 1.6 | 5.8 ± 0.5 |
| Nitrogen | | | | | | |
| (mg/dl) | | | | | | |
| Creatinine | 0.9 ± 0.1 | 1.2 ± 0.3 | 0.9 ± 0.2 | 0.9 ± 0.2 | 1.1 ± 0.4 | 1.1 ± 0.3 |
| (mg/dl) | | | | | | |

Coagulation Profile

| | | | | | | |
|---|---|---|---|---|---|---|
| Activated | 20.0 ± 0.0 | 20.3 ± 0.6 | 20.0 ± 0.0 | 21.8 ± 3.5 | 21.0 ± 1.4 | 20.0 ± 0.0 |
| Partial | | | | | | |
| Thromboplastin | | | | | | |
| Time (s) | | | | | | |
| Prothrombin | 11.1 ± 0.3 | 14.4 ± 0.4 | 10.2 ± 1.2 | 10.4 ± 1.1 | 10.7 ± 2.3 | 11.0 ± 0.8 |
| time (s) | | | | | | |
| Fibrinogen | 183.0 ± 100.8 | 209.7 ± 15.5 | 211.5 ± 90.9 | 189.8 ± 139.3 | 141.0 ± 21.2 | 172.5 ± 55.3 |
| (mg/dl) | | | | | | |

| Post- | | | | | | |
|---|---|---|---|---|---|---|
| operative | With Immunosuppression | | | | | |
| day | 1 | 3 | 7 | 10 | 15 | 20 |
| Complete Blood Count | | | | | | |
| Hemoglobin | 8.5 ± 3.1 | 8.5 ± 1.6 | 8.9 ± 0.7 | 9.0 ± 1.7 | 9.9 ± 3.0 | 8.5 ± 3.2 |
| (gm/dl) | | | | | | |
| White | 22.2 ± 12.6 | 15.2 ± 4.0 | 15.8 ± 6.1 | 16.8 ± 8.2 | 14.5 ± 5.0 | 12.4 ± 3.4 |
| Cells | | | | | | |
| (103/μ1) | | | | | | |
| Platelets | 132.0 ± 129.3 | 180.0 ± 66.0 | 251.3 ± 180.8 | 474.7 ± 397.7 | 718.5 ± 17.7 | 788.0 ± 181.0 |
| (103/μ1) | | | | | | |
| Liver Function | | | | | | |
| Total | 0.2 ± 0.0 | 0.2 ± 0.1 | 0.4 ± 0.2 | 0.2 ± 0.0 | 0.3 ± 0.1 | 0.2 ± 0.0 |
| Bilirubin | | | | | | |
| (mg/dl) | | | | | | |
| Aspartate | 142.0 ± 64.3 | 41.3 ± 29.6 | 49.0 ± 45.4 | 26.0 ± 3.5 | 24.5 ± 4.9 | 27.0 ± 4.2 |
| Aminotransferase | | | | | | |
| (IU/L) | | | | | | |
| Alanine | 69.3 ± 22.2 | 64.8 ± 23.5 | 51.5 ± 21.3 | 40.0 ± 14.2 | 36.0 ± 1.4 | 50.0 ± 11.3 |
| Aminotransferase | | | | | | |
| (IU/L) | | | | | | |
| Alkaline | 124.0 ± 53.2 | 117.5 ± 29.8 | 83.0 ± 12.4 | 64.7 ± 23.8 | 72.5 ± 0.7 | 73.5 ± 21.9 |
| Phosphatase | | | | | | |
| (IU/L) | | | | | | |
| Gamma-Glutamyl | 28.0 ± 6.3 | 32.0 ± 7.4 | 44.5 ± 14.2 | 38.3 ± 9.5 | 40.0 ± 2.8 | 35.5 ± 3.5 |
| Transferase | | | | | | |
| (IU/L) | | | | | | |
| Renal Function | | | | | | |
| Blood Urea | 17.0 ± 4.7 | 15.0 ± 2.6 | 10.3 ± 4.1 | 8.0 ± 3.6 | 12.5 ± 2.1 | 7.0 ± 1.4 |
| Nitrogen | | | | | | |
| (mg/dl) | | | | | | |
| Creatinine | 1.2 ± 0.3 | 013 ± 0.1 | 0.9 ± 0.2 | 0.8 ± 0.1 | 1.0 ± 0.1 | 0.9 ± 0.2 |
| (mg/dl) | | | | | | |
| Coagulation Profile | | | | | | |
| Activated | 20.2 ± 0.1 | 20.0 ± 0.0 | 21.0 ± 0.4 | 20.0 ± 0.0 | 20.0 ± 0.0 | 20.0 ± 0.0 |
| Partial | | | | | | |
| Thromboplastin | | | | | | |
| Time (s) | | | | | | |
| Prothrombin | 10.4 ± 1.6 | 10.1 ± 0.4 | 9.5 ± 0.5 | 9.6 ± 0.8 | 10.3 ± 0.1 | 10.1 ± 0.4 |
| time (s) | | | | | | |
| Fibrinogen | 146.7 ± 51.5 | 283.0 ± 60.3 | 205.3 ± 27.2 | 165.7 ± 60.7 | 122.0 ± 5.7 | 132.5 ± 9.9 |
| (mg/dl) | | | | | | |

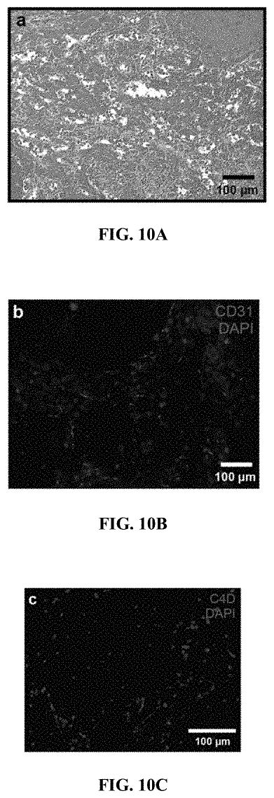

One rBEL was harvested from an immunosuppressed recipient animal at day 7 for histological analysis which demonstrated persistence of the HUVEC populations in the graft (FIG. 10A, FIG. 10B). The other 3 grafts were monitored by CT imaging until total loss of graft perfusion, which was observed on days 10, 15, and 20, respectively. Total loss of porto-venous flow was seen on days 10, 20 while one graft continued to have some portal-venous flow through the graft despite total loss of parenchymal perfusion (FIG. 8D). Evidence of rBEL perfusion was present in all of the grafts in the immunosuppressed group at day 7, demonstrating a significant increase in sustained perfusion over the non-immune suppressed group (p=0.01).

### Example 18—Early Immune Response to rBEL Xenotransplantation

The native pig immune response to HUVEC cells was characterized to confirm if the high rate of graft failure between Days 3 and 7 was associated with an immune response to the HUVECs used to revascularize the rBELs. Pig serum was collected at each CT scan and incubated with HUVEC cultures to perform a complement mediated cytotoxicity assay. Complement mediated cytotoxicity reaction was observed between naïve pig sera and HUVECs at baseline (range 30-85% cell death) demonstrating an inherent immune response to the human-derived cells without graft exposure in both no treatment and immunosuppressed groups (FIG. 8E). Evidence of an in vivo complement activation was observed by C4D deposition on endothelial cells in explanted rBEL samples (FIG. 10C).

Cytotoxicity significantly increased in the no treatment by Day 3 (81.7±21.0) (mean+/−s.d) and remained at >98% following Day 3. In contrast, immunosuppression significantly reduced cytotoxicity at Day 1 and Day 3 25.2 (±29.4), and 8.68 (±8.49) respectively, followed by a notable increase in cytotoxicity at day 7 84.7 (±13.0) and >98% cytotoxicity at Day 10 and Day 15 (FIG. 8E). rBEL perfusion in the no treatment and immunosuppression groups correlated to cytotoxicity responses. Decreased rBEL perfusion was preceded by a significant increase in an immune response as seen on Day 3 for no treatment and Day 7 for the immunosuppressed group (FIG. 8D, E).

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1.-3. (canceled)

4. An isolated at least partially recellularized liver comprising a perfusion decellularized extracellular matrix from a first animal and a plurality of endothelial cells from a second animal engrafted thereon; wherein prior to the recellularization, the perfusion decellularized extracellular matrix included a non-vasculature decellularized extracellular matrix and a vasculature decellularized extracellular matrix, and wherein the isolated at least partially recellularized liver comprises a greater expression level of LYVE-1 in a parenchymal niche of the isolated at least partially recellularized liver relative to an expression level of LYVE-1 in a large vessel of the isolated at least partially recellularized liver, as determined by isolating extraction of RNA from tissue of the isolated at least partially recellularized liver and quantitative reverse-transcriptase PCR.

5. The isolated at least partially recellularized liver of claim 4, wherein the perfusion decellularized matrix comprises a substantially intact exterior surface.

6. The isolated at least partially recellularized liver of claim 4, wherein the first animal is a mammal selected from the group consisting of a rodent, a pig, a monkey, a rabbit, a cow, a goat, a sheep, a dog, and a human.

7.-9. (canceled)

10. The isolated at least partially recellularized liver of claim 6, wherein the second mammal is a human.

11. (canceled)

12. The isolated at least partially recellularized liver of claim 10, wherein the endothelial cells are human umbilical vein endothelial cells (HUVEC).

13. The isolated at least partially recellularized liver of claim 4, further comprising a cannula.

14. The isolated at least partially recellularized liver of claim 4, wherein the isolated at least partially recellularized liver comprises a greater expression level of STAB-2 in a parenchymal niche of the isolated at least partially recellularized liver relative to an expression level of STAB-2 in a large vessel of the isolated at least partially recellularized liver, as determined by isolating extraction of RNA from tissue of the isolated at least partially recellularized liver and quantitative reverse-transcriptase PCR.

15. The isolated at least partially recellularized liver of claim 14, wherein the isolated at least partially recellularized liver in media has a 24 hour glucose consumption level of at least about 10 mg/hr, as determined by collecting the media and measuring the level of glucose using an electrochemical sensor.

16. A kit comprising the isolated at least partially recellularized liver of claim 4 in a sterile container.

17. A system comprising the isolated at least partially recellularized liver of claim 4, an input attached to the at isolated least partially recellularized liver, an output attached to the isolated at least partially recellularized liver, growth media, and at least one of: a temperature control apparatus, an atmosphere controlling apparatus, or a humidity controlling apparatus.

18. A cleanroom comprising the at isolated least partially recellularized liver of claim 4.

19. A factory comprising the isolated at least partially recellularized liver of claim 4.

20. A method comprising transplanting the at least partially recellularized liver of claim 4.

21. (canceled)

22. A method, comprising:
(a) providing a perfusion decellularized extracellular matrix of a decellularized mammalian liver in media,
(b) introducing a first solution comprising a population of endothelial cells to the perfusion decellularized extracellular matrix; such that at least some of the endothelial cells engraft on the at least a portion of the perfusion decellularized extracellular matrix, thereby providing a recellularized extracellular matrix of the decellularized mammalian liver,
(c) measuring a 24 hour glucose consumption level in a media of the endothelial cells engrafted on the recellularized extracellular matrix, and
(d) transplanting the recellularized extracellular matrix into a recipient when the 24 hour glucose level is at least about 10 mg/hr.

23. A method, comprising:
(a) administering to a recipient an immunosuppressor;
(b) introducing a first solution comprising a population of endothelial cells to a perfusion decellularized extracellular matrix; such that at least some of the endothelial cells engraft on at least a portion of the perfusion decellularized extracellular matrix, thereby providing a recellularized extracellular matrix of the decellularized mammalian liver; and
(c) transplanting the reendothelialized liver matrix into the recipient.

24.-45. (canceled)

46. A method of quality testing a recellularized liver, comprising: providing a recellularized liver, wherein the recellularized liver comprises a perfusion decellularized extracellular matrix and a population of endothelial cells engrafted thereon; determining a presence of a fenestration on the recellularized liver; detecting a level of glucose consumption within a 24 hour period; and designating the recellularized liver for further manufacture if the recellularized liver has a fenestration and a level of glucose consumption within a 24 hour period of at least about 10 mg/hr.
